(19) United States
(12) Patent Application Publication
(10) Pub. No.: US 2024/0254096 A1
(43) Pub. Date: Aug. 1, 2024

(54) ULTRAVIOLET AND VISIBLE RADIATION ABSORBING COMPOUNDS

(71) Applicant: ELKIMIA INC., Montreal (CA)

(72) Inventors: Samira OSATI, L'Ile-Bizard (CA); Elie ABOU-KHALIL, Montreal (CA)

(21) Appl. No.: 18/571,887

(22) PCT Filed: Jul. 6, 2022
(86) PCT No.: PCT/CA2022/051062

§ 371 Date: Dec. 19, 2023

Related U.S. Application Data
(60) Provisional application No. 63/219,176, filed on Jul. 7, 2021.

(51) Int. Cl. C07D 279/16; A61K 8/41; A61K 8/49; A61Q 17/04; C07C 251/20; C07D 249/08; C07D 417/12; C09D 5/32

(52) U.S. Cl. CPC C07D 279/16; A61K 8/411; A61K 8/494; A61K 8/496; A61Q 17/04; C07C 251/20; C07D 249/08; C07D 417/12; C09D 5/32; A61K 2800/40

(57) ABSTRACT

The present technology relates to UV radiation-absorbing and/or visible radiation-absorbing compounds, and use of same in the preparation of compositions for protecting biological and nonbiological material against UV and/or visible radiation. Specifically, the compounds of the present technology may be used in the preparation of compositions for protecting biological or non-biological material against UV radiation, in particular UVA, UVB, UVC or visible radiation, or any combinations thereof. The biological material can be skin and the composition and/or the formulation can be a cosmetic or personal care composition, such as a sunscreen composition. The non-biological material can be an article of manufacture such as a textile or fabric.

### FIELD OF TECHNOLOGY

The present technology generally relates to compounds which absorb ultraviolet (UV) radiation and visible light and protect biological materials as well as non-biological materials from damaging exposure to UV radiation and visible light, as well as compositions comprising same.

### BACKGROUND

Commercially available ultraviolet blocking agents typically include compounds such as para-aminobenzoic acid derivatives, benzotriazoles, benzophenones, methoxycinnamates and salicylates. Mycosporine-like amino acids (MAAs) have also been identified as ultraviolet-absorbing agents. MAAs are small molecules of about 400 Da produced by organisms that live in environments with high volumes of sunlight, typically marine environments. The structures of over 30 MAAs have been resolved and they contain a central cyclohexenone or cyclohexenimine ring as well as a wide variety of substitutions. The ring structure is thought to absorb ultraviolet light and accommodate free radicals. MAAs absorb ultraviolet light, typically between 310 nm and 360 nm. It is this light absorbing property that allows MAAs to protect cells from harmful ultraviolet radiation. Biosynthetic pathways of specific MAAs depend on the specific MAA and the organism that is producing it. These biosynthetic pathways often share common enzymes and intermediates with other major biosynthetic pathways.

Useful ultraviolet absorbing agents such as the ones mentioned above must meet various criteria including stability, acceptable permanence, efficacy, compatibility with the media with which they are to be mixed or be incorporated into, non-toxicity and not harmful to the surface onto which they are to be applied. These criteria limit the choice of ultraviolet protecting agents available to be used in various applications. Some such agents are described in U.S. Pat. No. 9,487,474, incorporated herein by reference.

Visible radiation (visible light) can also present negative effects. For example, visible light radiation can exert various biologic effects such as erythema, pigmentation, thermal damage and free radical production. Additionally, visible light exposure can cause or exacerbate photodermatoses such as solar urticaria, chronic actinic dermatosis (CAD) and cutaneous porphyrias.

Therefore, there remains a need in the art for additional agents that absorb both ultraviolet radiations as well as visible radiations and that protect biological and non-biological materials against the harmful damages caused by ultraviolet radiations and visible radiation.

### SUMMARY OF DISCLOSURE

The shortcomings of the prior art are generally mitigated by new compounds that absorb UV radiation and/or visible radiation and protect biological materials as well as non-biological materials from damaging exposure to UV radiation and/or visible radiations.

From one aspect, the present technology relates to compounds having formula I or an acceptable salt thereof:

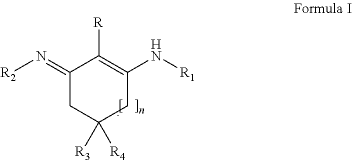

wherein R₁, is an alkyl; an alkene; an alkyne; an aryl; a heterocycle; a cycloalkyl; an alkoxy; an alkanoyl; an arylalkyl; a carboxyl; a heteroaryl; a heteroarylalkyl; a phenyl; a benzyl; a hydroxyl; a carboxylic acid; an ester; a sulfinyl; a sulfhydryl; a sulfide; a sulfonyl; a sulfino; a phosphino; a phosphono; a phosphate; an amine; a halo; a carboxamide, or R₁ forms a heterocycle with R₅;
R₂ is an alkyl; an alkene; an alkyne; an aryl; a heterocycle; a cycloalkyl; an alkoxy; an alkanoyl; a hydroxyl; a halo; a phenyl; a benzyl; a carboxylic acid or an ester group, wherein R₂ comprises at least one nitrogen atom;
R₃ and R₄ are each independently a hydrogen; an alkyl; an alkene; an alkyne; an aryl; a heterocycle; a cycloalkyl; an alkoxy; an alkanoyl; a sulfo group; a hydroxyl group; a phosphono group; an ester group; a carboxylic acid group; or a phenyl group;
R₅ is a hydrogen; an alkyl; an alkene; an alkyne; an aryl; a heterocycle; a cycloalkyl; an alkoxy; an alkanoyl; an alkynyl; a hydroxyl; a sulfo group; a halo group; a phosphono group; an ester group; a carboxylic acid group; a phenyl group; an alkyl fatty acid chain or polyether; and
n is 1, 2, 3 or 4.

From another aspect, the present technology relates to a compound having formula 3,

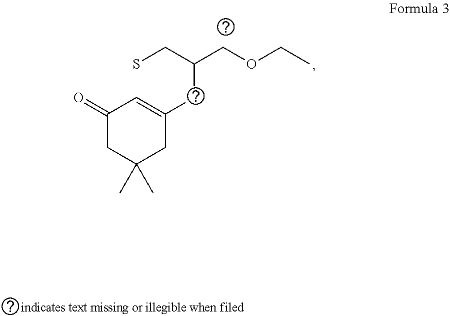 [SMILES: CCOCC(CS)CC1=CC(=O)CC(C)(C)C1]

which absorbs ultraviolet radiation and/or visible radiation.

From another aspect, the present technology relates to a compound of formula 66,

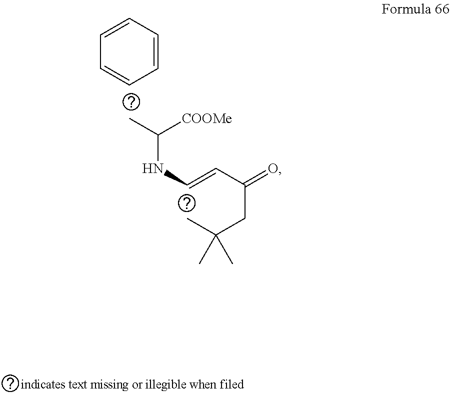

which absorbs ultraviolet radiation and/or visible radiation.

From another aspect, the present technology relates to a compound of formula 70,

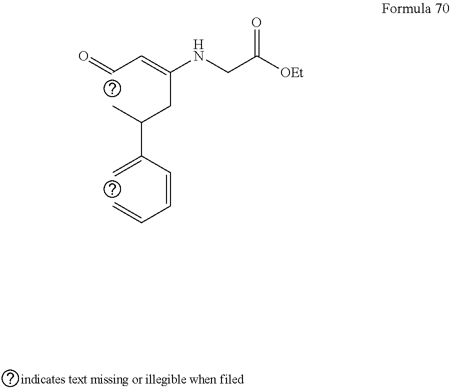 [SMILES: C=C/C=C\C(=C)C(C)C/C(=C\C=O)NCC(=O)OCC]

which absorbs ultraviolet radiation and/or visible radiation.

From another aspect, the present technology relates to a compound of formula 72:

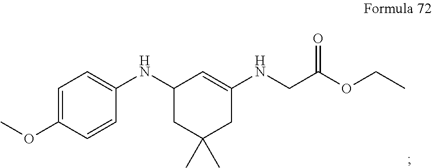 [SMILES: CCOC(=O)CNC1=CC(Nc2ccc(OC)cc2)CC(C)(C)C1]

which absorbs ultraviolet radiation and/or visible radiation.

From another aspect, the present technology relates to a compound of formula 73:

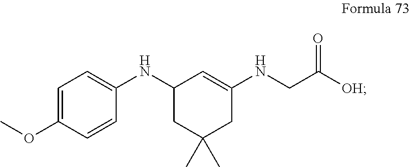 [SMILES: COc1ccc(NC2C=C(NCC(=O)O)CC(C)(C)C2)cc1]

which absorbs ultraviolet radiation and/or visible radiation.

From another aspect, the present technology relates to a compound of formula 74:

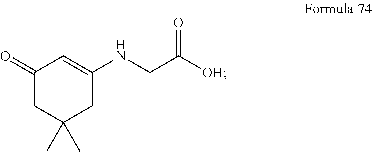

which absorbs ultraviolet radiation and/or visible radiation.

From another aspect, the present technology relates to a compound of formula 75:

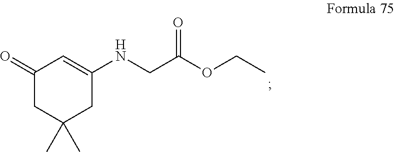 [SMILES: CCOC(=O)CNC1=CC(=O)CC(C)(C)C1]

which absorbs ultraviolet radiation and/or visible radiation.

From another aspect, the present technology relates to a compound of formula 76:

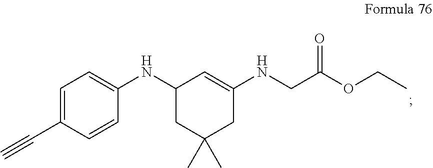 [SMILES: C#Cc1ccc(NC2C=C(NCC(=O)OCC)CC(C)(C)C2)cc1]

which absorbs ultraviolet radiation and/or visible radiation.

From another aspect, the present technology relates to a compound of formula 77:

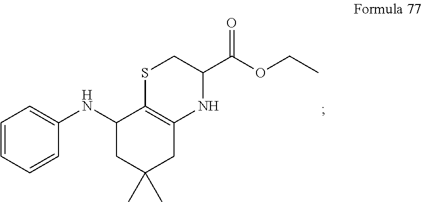 [SMILES: CCOC(=O)C1CSC2=C(CC(C)(C)CC2Nc2ccccc2)N1]

which absorbs ultraviolet radiation and/or visible radiation.

From another aspect, the present technology relates to the use of said compounds or any combinations thereof in the preparation of a composition for protecting a biological or non-biological material against ultraviolet radiation and/or visible radiation.

From another aspect, the present technology relates to methods for protecting a surface of a biological or non-biological material against UV radiation and/or visible radiation comprising applying to the surface of said biological or non-biological material a composition comprising the compounds of the present technology.

Other aspects and features of the present disclosure will become apparent to those ordinarily skilled in the art upon review of the following description of specific embodiments in conjunction with the accompanying drawings.

### BRIEF DESCRIPTION OF THE DRAWINGS

All features of embodiments which are described in this disclosure are not mutually exclusive and can be combined with one another. For example, elements of one embodiment can be utilized in the other embodiments without further mention. A detailed description of specific embodiments is provided herein below with reference to the accompanying drawings in which:

FIG. 1 illustrates the absorption spectrums of the compounds of formula 12, 14, 16, 18 and 21 (C #12, C #14, C #16, C #18, and C #21 respectively).

FIG. 2 illustrates the absorption spectrums of the compounds of formula 28, 30, 32, and 35 (C #28, C #30, C #32, and C #35 respectively).

FIG. 3 illustrates the absorption spectrums of the compounds of formula 74, 73, 24, 25, 26, 27, 29, 31 and 75 (C #74, C #73, C #24, C #25, C #26, C #27, C #29, C #31, and C #75 respectively).

FIG. 4 illustrates the maximum absorption of various compounds of the present technology.

FIG. 5 illustrates the photostability of a compound according to certain embodiments of the present technology before irradiation, and after 24 hours and 48 h of irradiation.

FIG. 6 illustrates the thermogravimetric analysis (TGA) of various compounds of the present technology.

FIG. 7A illustrates the foaming of a water-based composition according to certain embodiments of the present technology after mixing with a high shear mixer. FIG. 7B illustrates the foaming of the water-based composition of FIG. 7A in the presence of a defoamer. FIG. 7C illustrates the stability of the composition of FIG. 7B after two weeks.

FIG. 8 illustrates a protocol for the preparation of a coating composition according to certain embodiments of the present technology used for dyeing of a textile and application of same to the textile.

FIG. 9 is a photograph of textiles prepared by extrusion according to certain embodiments of the present technology.

FIG. 10 is a photograph of a textile prepared by extrusion according to certain embodiments of the present technology before and after aging for 40 h in a Xenon chamber.

### DETAILED DISCLOSURE OF EMBODIMENTS

The present technology is explained in greater detail below. This description is not intended to be a detailed catalog of all the different ways in which the technology may be implemented, or all the features that may be added to the instant technology. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which variations and additions do not depart from the present technology. Hence, the following description is intended to illustrate some particular embodiments of the technology, and not to exhaustively specify all permutations, combinations and variations thereof.

As used herein, the singular form “a,” “an” and “the” include plural referents unless the context clearly dictates otherwise.

The recitation herein of numerical ranges by endpoints is intended to include all numbers subsumed within that range (e.g., a recitation of 1 to 5 includes 1, 1.5, 2, 2.75, 3, 3.80, 4, 4.32, and 5).

The term “about” is used herein explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. For example, the term “about” in the context of a given value or range refers to a value or range that is within 20%, preferably within 15%, more preferably within 10%, more preferably within 9%, more preferably within 8%, more preferably within 7%, more preferably within 6%, and more preferably within 5% of the given value or range.

The expression “and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

As used herein, the term “comprise” is used in its non-limiting sense to mean that items following the word are included, but items not specifically mentioned are not excluded.

As used herein, the terms “compound” and “compound(s) of the invention” are used interchangeably to refer to any compounds, including acceptable salts, hydrates or solvates thereof, disclosed herein specifically or generically.

The expression “biological materials”, as used herein, unless otherwise indicated, is intended to include humans, animals and plants and includes for example: cells, hair, skin, as well as other human and animal tissues. The expression “non-biological materials”, as used herein, unless otherwise indicated, is intended to include all things that do not fall into the definition of “biological materials”.

The expression “solar radiation”, as used herein, unless otherwise indicated, is intended to include the total frequency spectrum of electromagnetic radiation given off by the sun, including radio waves, X-rays, infrared, visible, and ultraviolet.

The terms “ultraviolet” and “UV”, as used herein, unless otherwise indicated, are intended to mean ultraviolet or ultraviolet light. UV in the electromagnetic radiation with a wavelength shorter than that of visible light, but longer than X-rays, in the range of about 10 nm to about 400 nm, and energies from about 3 eV to about 124 eV (the abbreviation “eV, herein refers to electron volts). Ultraviolet A (UVA) refers to UV radiation in the spectrum of between 320-400 nm, it is also referred to as “longer” rays. The UVA waveband is further divided into UVA I (340-400 nm) and UVA II (320-340 nm). UVA are the principal cause of long-term skin damage due to the sun and may also contribute to sunburn. Ultraviolet B (UVB) refers to radiation in the spectrum of 290-320 nm, it is also referred to as “shorter” rays. UVB rays are the principal cause of sunburn due to sun exposure. Ultraviolet C (UVC) refers to radiation in the spectrum of between about 200 nm and about 280 nm. UVC has germicidal applications and is commonly used for the decontamination of textiles. Ultraviolet-visible (UV-vis) refers to radiation comprising UV radiation (between about 100 nm and about 400 nm) and visible radiation (between about 400 nm and about 800 nm).

As used herein, the expression “visible radiation” or “visible light” refers to electromagnetic radiation with a wavelength ranging between about 400 nm and about 750 nm.

The term “imine” or “imino”, as used herein, unless otherwise indicated, includes a functional group or chemical compound containing a carbon-nitrogen double bond. The expression “imino compound”, as used herein, unless otherwise indicated, refers to a compound that includes an “imine” or an “imino” group as defined herein.

The term “hydroxyl”, as used herein, unless otherwise indicated, includes —OH. The terms “halogen” and “halo”, as used herein, unless otherwise indicated, include a chlorine, chloro, Cl; fluorine, fluoro, F, bromine, bromo, Br; or iodine, iodo, I.

The term “aryl”, as used herein, unless otherwise indicated, includes a carbocyclic aromatic group. Examples of aryl groups include, but are not limited to, phenyl, benzyl, naphthyl and anthracenyl.

The terms “amine” and “amino”, as used herein, unless otherwise indicated, include a functional group that contains a nitrogen atom with a lone pair of electrons and wherein one or more hydrogen atoms have been replaced by a substituent such as, but not limited to, an alkyl group or an aryl group.

The term “alkyl” as used herein, unless otherwise indicated, includes saturated monovalent hydrocarbon radicals having straight or branched moieties, such as but not limited to, methyl, ethyl, propyl, butyl, pentyl, hexyl, octyl groups, etc. Representative straight-chain lower alkyl groups include, but are not limited to, -methyl, -ethyl, -n-propyl. -n-butyl, -n-pentyl, -n-hexyl, -n-heptyl and -n-octyl, while branched lower alkyl groups include, but are not limited to, -isopropyl, -sec-butyl, -isobutyl, -tert-butyl, -isopentyl, 2-methylbutyl, 2-methylpentyl, 3-methylpentyl, 2,2-dimethylbutyl, 2,3-dimethylbutyl, 2,2-dimethylpentyl, 2,3-dimethylpentyl, 3,3-dimethylpenty 1,2,3,4-trimethylpentyl, 3-methylhexyl, 2,2-dimethylhexyl, 2,4-dimethylhexyl, 2,5-dimethylhexyl, 3,5-dimethylhexyl, 2,4-dimethylpentyl, 2-methylheptyl, 3-methylheptyl, unsaturated C—Cs alkyls include, but are not limited to, -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, 1-hexyl, 2-hexyl, 3-hexyl, -acetylenyl, -propynyl, -1-butynyl, -2-butynyl, -1-pentynyl, -2-pentynyl, -3-methyl-1 butynyl.

The term “carboxyl”, as used herein, unless otherwise indicated, includes a functional group consisting of a carbon atom double bonded to an oxygen atom and single bonded to a hydroxyl group (—COOH).

The term “alkenyl”, as used herein, unless otherwise indicated, includes alkyl moieties having at least one carbon-carbon double bond wherein alkyl is as defined above and including E and Z isomers of said alkenyl moiety.

The term “alkynyl”, as used herein, unless otherwise indicated, includes alkyl moieties having at least one carbon-carbon triple bond wherein alkyl is as defined above. The term “acyl”, as used herein, unless otherwise indicated, includes a functional group derived from an aliphatic carboxylic acid, by removal of the hydroxyl (—OH) group.

The term “alkoxyl”, as used herein, unless otherwise indicated, includes O-alkyl groups wherein alkyl is as defined herein, and O represents oxygen. Representative alkoxyl groups include, but are not limited to —O-methyl, —O-ethyl, —O-n-propyl. —O-n-butyl, —O-n-pentyl, —O-n-hexyl, —O-n-heptyl, —O-n-octyl, —O-isopropyl. —O-sec-butyl, —O-isobutyl, —O-tert-butyl, —O-isopentyl, —O-2-methylbutyl, —O-2-methylpentyl, —O-3-methyl pentyl, —O-2,2-dimethylbutyl, —O-2,3-dimethylbutyl, —O-2,2-dimethylpentyl, —O-2,3-dimethylpentyl, —O-3,3-dimethylpentyl, —O-2,3,4-trimethylpentyl, —O-3-methyl hexyl, —O-2,2-dimethylhexyl, —O-2,4-dimethylhexyl, —O-2,5-dimethylhexyl, —O-3,5-dimethylhexyl, —O-2, 4,dimethylpentyl, O-2-methylheptyl, —O-3-methylheptyl, —O-vinyl, —O-allyl, —O-1-butenyl, O-2-butenyl, -0-isobutylenyl, —O-1-pentenyl, —O-2-pentenyl, —O-3-methyl-1-butenyl, —O-2-methyl-2-butenyl, —O-2,3-dimethyl-2-butenyl, —O-1-hexyl, —O-2-hexyl, —O-3-hexyl, —O-acetylenyl, —O-propynyl, —O-1-butynyl, —O-2-butynyl, —O-1-pentynyl, —O-2-pentynyl and —O-3-methyl 1-butynyl, —O-cyclopropyl, —O-cyclobutyl, —O-cyclopentyl, —O-cyclohexyl, —O-cycloheptyl, —O-cyclooctyl, —O-cyclononyl and —O-cyclodecyl, —O—CH₂-cyclopropyl, —O—CH-cyclobutyl, —O—CH-cyclopentyl, —O—CH₂-cyclohexyl, —O—CH₂-cycloheptyl, —O—CH₂-cyclooctyl, —O—CH₂-cyclononyl, —O—CH₂-cyclodecyl, —O—(CH₂)₂-cyclopropyl, —O—(CH₂)₂-cyclobutyl, —O—(CH₂)₂-cyclopentyl, —O—(CH₂)₂-cyclohexyl, —O—(CH₂)₂-cycloheptyl, —O—(CH₂)₂-cyclooctyl, —O—(CH₂)₂-cyclononyl and —O—(CH₂)₂-cyclodecyl.

The term “cycloalkyl”, as used herein, unless otherwise indicated, includes a non-aromatic, saturated or partially saturated, monocyclic or fused, spiro or unfused bicyclic or tricyclic hydrocarbon referred to herein containing a total of from 3 to 10 carbon atoms, preferably 3 to 8 ring carbon atoms. Examples of cycloalkyls include, but are not limited to, C—Cs cycloalkyl groups include, but are not limited to, -cyclopropyl, -cyclobutyl, -cyclopentyl, -cyclopentadienyl, -cyclohexyl, -cyclohexenyl, -1,3-cyclohexadienyl, -1,4-cyclohexadienyl, -cycloheptyl, -1,3-cycloheptadienyl, -1,3,5-cycloheptatrienyl, -cyclooctyl, and -cyclooctadienyl.

The term “cycloalkyl” also includes -lower alkyl-cycloalkyl, wherein lower alkyl and cycloalkyl are as defined herein. Examples of -lower alkyl-cycloalkyl groups include but are not limited to, —CH₂-cyclopropyl, —CH₂-cyclobutyl, —CH₂-cyclopentyl, —CH₂-cyclopentadienyl, —CH₂ cyclohexyl, —CH₂— cycloheptyl and —CH₂-cyclooctyl.

The term “heterocyclic”, as used herein, unless otherwise indicated, includes an aromatic or non-aromatic cycloalkyl in which one to four of the ring carbon atoms are independently replaced with a heteroatom from the group consisting of O, S and N. Representative examples of a heterocycle include, but are not limited to, benzofuranyl, benzothiophene, indolyl, benzopyrazolyl, coumarinyl, isoquinolinyl, pyrrolyl pyrrolidinyl, thiophenyl, furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl, (1,4)-dioxane, (1,3)-dioxolane, 4,5-dihydro-1H imidazolyl and tetrazolyl. Heterocycles can be substituted or unsubstituted. Heterocycles can also be bonded at any ring atom (i.e., at any carbon atom or heteroatom of the hetero cyclic ring).

The term “cyano”, as used herein, unless otherwise indicated, includes a —CN group.

The term “alcohol”, as used herein, unless otherwise indicated, includes a compound in which the hydroxyl functional group (—OH) is bound to a carbon atom. In particular, this carbon center should be saturated, having single bonds to three other atoms.

The term “solvate” is intended to mean a solvate form of a specified compound that retains the effectiveness of such compound. Examples of solvates include compounds of the invention in combination with, for example: water, isopropanol, ethanol, methanol, dimethylsulfoxide (DMSO), ethyl acetate, acetic acid, or ethanolamine.

The term “mmol”, as used herein, is intended to mean millimole.

The term “equiv”, as used herein, is intended to mean equivalent.

The term “mL, as used herein, is intended to mean milliliter.

The term “g”, as used herein, is intended to mean gram.

The term “kg”, as used herein, is intended to mean kilogram.

The term “μg”, as used herein, is intended to mean micrograms.

The term “h”, as used herein, is intended to mean hour.

The term “min”, as used herein, is intended to mean minute.

The term “M”, as used herein, is intended to mean molar.

The term “μL”, as used herein, is intended to mean microliter.

The term “μM”, as used herein, is intended to mean micromolar.

The term “nM”, as used herein, is intended to mean nanomolar.

The term “N”, as used herein, is intended to mean normal.

The term “amu”, as used herein, is intended to mean atomic mass unit.

The term “C.”, as used herein, is intended to mean degree Celsius.

The term “wt/wt”, as used herein, is intended to mean weight/weight.

The term “v/v”, as used herein, is intended to mean volume/volume.

The term “MS”, as used herein, is intended to mean mass spectroscopy.

The term “HPLC”, as used herein, is intended to mean high performance liquid chromatograph.

The term “RT”, as used herein, is intended to mean room temperature.

As used herein, the expression “acceptable salt” refers to acceptable organic or inorganic salts of a compound of the invention. Preferred salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate. succinate, maleate, gentisinate, fumarate, gluconate, glucaronate, saccharate, formate, benzoate, glutamate, methanesulfonate, ethanesulfonate, benzenesulfonate, p-toluenesulfonate, and pamoate (i.e., 1,1′-methylene-bis (2-hydroxy-3-naphthoate)) salts. An acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counterions. Hence, a pharmaceutically acceptable salt can have one or more charged atoms and/or one or more counterion.

### Compounds of the Present Technology

From one aspect there is provided a compound having formula I or an acceptable salt thereof which absorbs ultraviolet radiation, such as UVA, UVB, or UVC and/or visible radiation or any combination thereof:

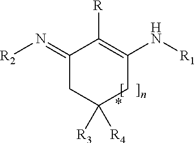

wherein R₁, is an alkyl; an alkene; an alkyne; an aryl; a heterocycle; a cycloalkyl; an alkoxy; an alkanoyl; an arylalkyl; a carboxyl; a heteroaryl; a heteroarylalkyl; a phenyl; a benzyl; a hydroxyl; a carboxylic acid; an ester; a sulfinyl; a sulfhydryl; a sulfide; a sulfonyl; a sulfino; a phosphino; a phosphono; a phosphate; an amine; a halo; a carboxamide, or wherein R₁ forms a heterocycle with R₅;
R₂ is an alkyl; an alkene; an alkyne; an aryl; a heterocycle; a cycloalkyl; an alkoxy; an alkanoyl; a hydroxyl; a halo; a phenyl; a benzyl; a carboxylic acid or an ester group, wherein R₂ comprises at least one nitrogen atom;
R₃ and R₄ are each independently a hydrogen; an alkyl; an alkene; an alkyne; an aryl; a heterocycle; a cycloalkyl; an alkoxy; an alkanoyl; a sulfo group; a hydroxyl group; a phosphono group; an ester group; a carboxylic acid group; or a phenyl group;
R₅ is a hydrogen; an alkyl; an alkene; an alkyne; an aryl; a heterocycle; a cycloalkyl; an alkoxy; an alkanoyl; a alkynyl; a hydroxyl; a sulfo group; a halo group; a phosphono group; an ester group; a carboxylic acid group; a phenyl group; an alkyl fatty acid chain or polyether; and
n is 1, 2, 3 or 4.

In certain embodiments the compound has the following formulas:

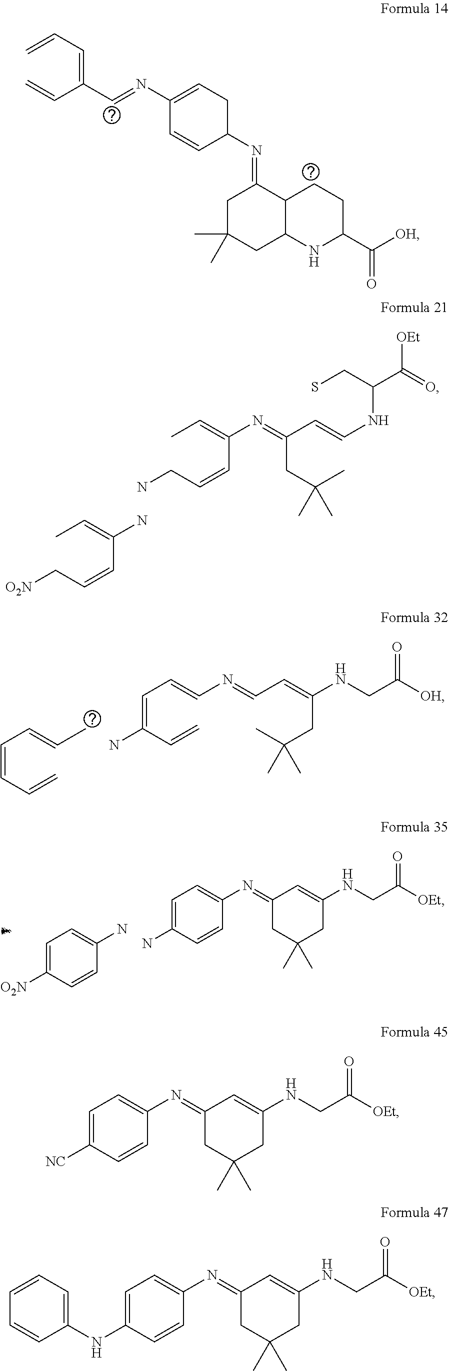

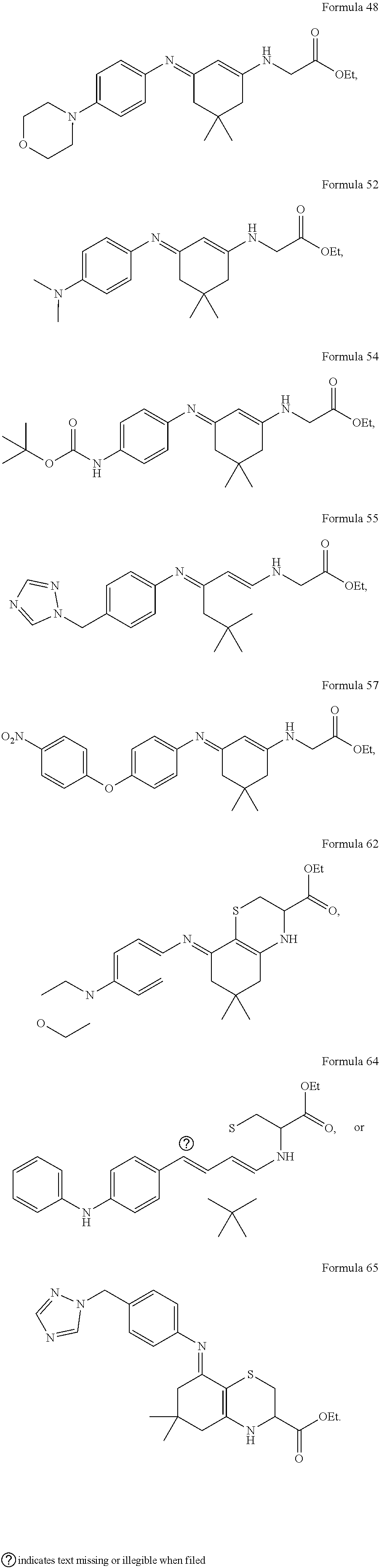

In other embodiments, the compound is of formula 3:

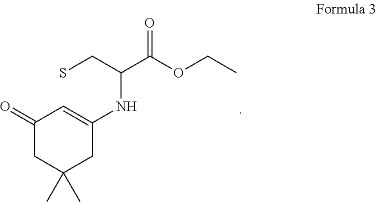 [SMILES: CCOC(=O)C(CS)NC1=CC(=O)CC(C)(C)C1]

In further embodiments, the compound is of formula 66:

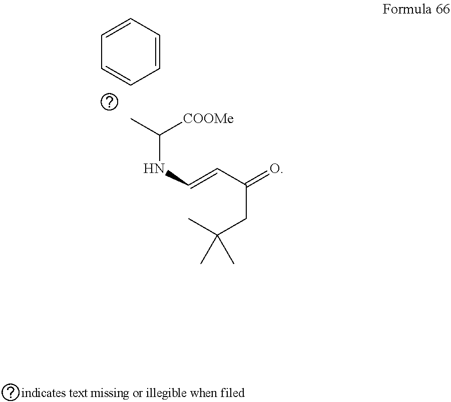

In yet further embodiments, the compound is of formula 70:

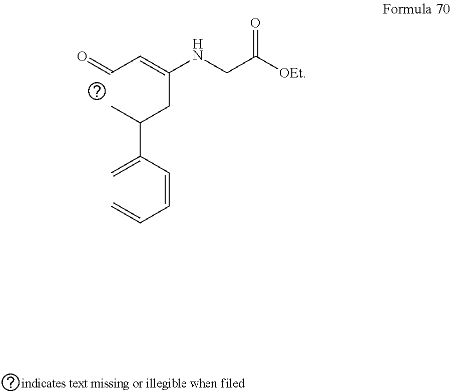 [SMILES: C=C/C=C\C(=C)C(C)C/C(=C\C=O)NCC(=O)OCC]

In other embodiments, the compound is of formula 72:

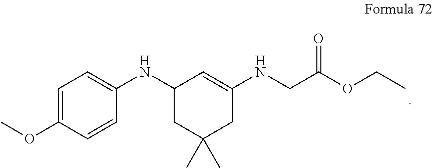 [SMILES: CCOC(=O)CNC1=CC(Nc2ccc(OC)cc2)CC(C)(C)C1]

In yet other embodiments, the compound is of formula 73:

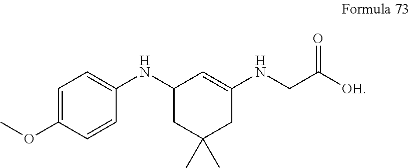 [SMILES: COc1ccc(NC2C=C(NCC(=O)O)CC(C)(C)C2)cc1]

In further embodiments, the compound is of formula 74:

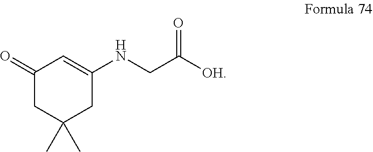

In yet further embodiments, the compound is of formula 75:

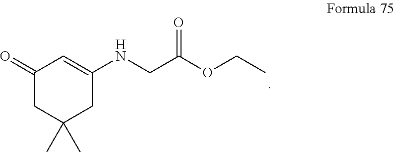 [SMILES: CCOC(=O)CNC1=CC(=O)CC(C)(C)C1]

In other embodiments, the compound is of formula 76:

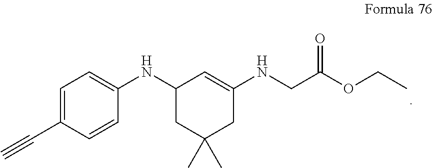 [SMILES: C#Cc1ccc(NC2C=C(NCC(=O)OCC)CC(C)(C)C2)cc1]

In yet another embodiment, the compound is of formula 77:

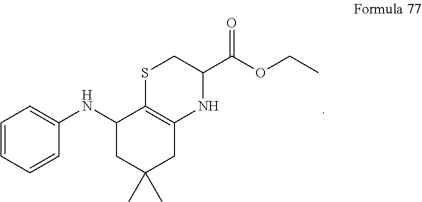 [SMILES: CCOC(=O)C1CSC2=C(CC(C)(C)CC2Nc2ccccc2)N1]

### Uses and Methods

From another aspect, the present technology relates to the use of the compounds disclosed herein in protection of biological and non-biological material against ultraviolet radiation and/or visible radiation. Specifically, the compounds of the present technology may be used in the preparation of compositions for protecting biological or non-biological material against UV radiation, in particular UVA, UVB, UVC or visible radiation, or any combinations thereof.

In certain embodiments, depending on the targeted radiation, the compounds of the present technology may be selected to be incorporated alone or in different combinations in said compositions to guarantee maximum protection over a certain radiation spectrum. Said compositions may therefore comprise at least one, two, three or more of the compounds of the present technology.

In certain embodiments, the compounds of the present technology may be incorporated into the compositions in an amount of at least about 0.1%, or from about 0.1% to about 25 w %, from about 0.1% to about 15%, from about 0.1% to about 10%, from about 0.1% to about 5%, from about 0.1% to about 2%, from about 5 w % to about 20 w %, from about 8 w % to about 15%, from about 8 w % to about 12 w %, about 1%, or about 10 w % of the total weight of the composition.

Said compositions may further comprise other compounds in order to obtain formulations and/or compositions with desired characteristics as discussed in greater detail below. Such other compounds may include a wide range of ingredients and compounds that are not UV absorbers/filters/blockers per se but that help to control characteristics of the composition itself such as film thickness, opacity, rub resistance, water proofing and uniformity. Alternatively, such other compounds may also include a wide range of ingredients that act as UV absorbers/filters/blockers, such as compounds that are UVA absorbers/filters/blockers and compounds that are UVB absorbers/filters/blockers.

### Cosmetic or Personal Care Compositions and or Formulations

In some embodiments, the compounds of the present technology are used in the preparation of compositions and/or formulations for protecting biological materials from UV and/or visible radiations. In certain embodiments, the biological material may be skin and the composition and/or the formulation may be a cosmetic or personal care composition, such as a sunscreen composition. Such compositions may be formulated according to techniques well known in the art, such as techniques for preparation of oil-in-water or water-in-oil emulsions. In addition, the compounds of the present technology may be formulated into carriers such as, water, water-based liquids, lotions, dispersions, oils, oil-based solutions, powder, gels, emulsions, dispersions or mixtures thereof. The appropriate amount of carrier can readily be determined by those skilled in the art according to, for example, a desired sun protection factor (SPF) to achieve. The specific amount of compounds disclosed herein needed to obtain a desired sun protection factor (SPF) can be determined by techniques well known in the art. Sunscreen should provide a minimum protection against UVA and/or UVB rays. In some embodiments, an increased sun protection factor (i.e., mainly UVB protection) includes an increase in the UVA protection as well. In other embodiments, the protection against UVA and UVB radiation are related. In further embodiments, the sunscreen may provide protection against UVA and UVB. In yet further embodiments, the sunscreen may provide protection against UVA, UVB and visible radiation.

The UV absorbance of a sunscreen product can be determined in vitro over the entire UV spectrum (290 nm-400 nm) using substrate spectrophotometry. For example, a uniform amount and thickness of sunscreen is applied to a slide and exposed to UV light; the absorbance of that UV radiation is measured according to techniques well known in the art. The UV absorbance curve obtained demonstrates the amplitude and breadth of protection provided (from 290 nm-400 nm) across the UV spectrum. The “amplitude” of the absorbance curve reflects the degree of protection. The higher the amplitude of the curve, the greater the absorbance and the more protection provided at that wavelength. Within the UVB portion of the spectrum (290 nm-320 nm) this amplitude correlates with the SPF. The greater the “breadth” of the curve, the more protection provided against longer wave UV radiation. In other words, the greater the “breadth” of the curve, the broader the spectrum of sun protection provided. Mathematical integration of the measured spectral absorbance from 290 nm to 400 nm is performed to calculate the area beneath the curve. The “Critical Wavelength” (λc) is the wavelength below which 90% of the area under the absorbance curve resides. A SPF value of 2 generally absorbs 50% UVB, a SPF value of 15 generally absorbs 93.3% UVB, SPF 30 absorbs 96.7% UVB and SPF 50 absorbs 98% UVB.

In the preparation of a sunscreen composition, the compounds defined herein may be used in combination with other UV-absorbing agents known in the art, such as, but not limited to, UV-blocking agents hydrophilic or lipophilic organic UV-A and/or UV-B sunscreen agents. Examples of other UV-absorbing agents which may be included in the formulations and/or compositions of the present technology include, but are not limited to: aminobenzoic acid; padimate O; phenylbenzimidazole sulfonic acid; cinoxate, dioxybenzone; oxybenzone; homosalate; menthyl anthranilate, octocrylene; octyl methoxycinnamate; octyl salicylate; sulisobenzone; trolamine salicylate; avobenzone; ecamsule; titanium dioxide; 4-methylbenzylidene camphor; tinosorb M; tinosorb S; neo heliopan AP; mexoryl XL; benzophenone-9; uvinul T 150; uvinul A Plus; uasorb HEB; parsol SLX and isopentenyl-4-methoxycinnamate; 4-dimethylaminobenzoic acid 2-ethylhexyl ester; salicylic acid derivatives, for example salicylic acid 2-ethylhexyl ester; benzophenone derivatives, for example 2-hydroxy-4-methoxybenzophenone and its 5-sulfonic acid derivative; dibenzoylmethane derivatives, for example 1-(4-tert-butylphenyl)-3-(4-methoxyphenyl)-propane-1,3-dione; diphenylacrylates, for example 2-ethylhexyl-2-cyano-3,3-diphenylacrylate, and 3-(benzofuranyl)-2-cyanoacrylate; 3-imidazol-4-ylacrylic acid and esters; benzofuran derivatives, such as 2-(p-aminophenyl)benzofuran derivatives; polymeric UV absorbers, such as benzylidene malonate derivatives; cinnamic acid derivatives, for example the 4-methoxycinnamic acid 2-ethylhexyl ester and isoamyl ester or cinnamic acid derivatives; camphor derivatives, for example 3-(4′-methyl)benzylidene-bornan-2-one, 3-benzylidene-bornan-2-one, N-[2(and 4)-2-oxyborn-3-ylidene-methyl)-benzyl]acrylamide polymer, 3-(4′-trimethylammonium)-benzylidene-bornan-2-one methyl sulfate, 3,3′-(1,4-phenylenedimethine)-bis(7,7-dimethyl-2-oxo-bicyclo[2,2,1]heptane-1-methane-sulfonic acid) and salts, 3-(4′-sulfo)benzylidene-bornan-2-one and salts; camphorbenzalkonium methosulfate; hydroxyphenyltriazine compounds, for example 2-(4′-methoxyphenyl)-4,6-bis(2′-hydroxy-4′-n-octyloxyphenyl)-1,3,5-triazine; 2,4-bis{[4-(3-(2-propyloxy)-2-hydroxy-propyloxy)-2-hydroxy]-phenyl}-6-(4-methoxyphenyl)-1,3,5-triazine; 2,4-bis{[4-(2-ethyl-hexyloxy)-2-hydroxy]-phenyl}-6-[4-(2-methoxyethyl-carboxyl)-phenylamino]-1,3,5-triazine; 2,4-bis{[4-(tris(trimethylsilyloxy-silylpropyloxy)-2-hydroxy]-phenyl}-6-(4-methoxyphenyl)-1,3,5-triazine; 2,4-bis{[4-(2″-methylpropenyloxy)-2-hydroxy]-phenyl}-6-(4-methoxyphenyl)-1,3,5-triazine; 2,4-bis{[4-(1′,1′,1′,3′,5′,5′,5′-heptamethyltrisilyl-2″-methyl-propyloxy)-2-hydroxy]-phenyl}-6-(4-methoxyphenyl)-1,3,5-triazine; 2,4-bis{[4-(3-(2-propyloxy)-2-hydroxy-propyloxy)-2-hydroxy]-phenyl}-6-[4-ethylcarboxy)-phenylamino]-1,3,5-triazine; benzotriazole compounds, for example 2,2′-methylene-bis(6-(2H-benzotriazol-2-yl)-4-(1,1,3,3-tetramethylbutyl)-phenol; trianilino-s-triazine derivatives, for example 2,4,6-trianiline-(p-carbo-2′-ethyl-1′-oxy)-1,3,5-triazine; 2-phenylbenzimidazole-5-sulfonic acid and salts thereof; menthyl-o-aminobenzoates; physical sunscreens coated or not coated, such as titanium dioxide, zinc oxide, iron oxides, mica, MnO, Fe₂O₃, Ce₂O₃, Al₂O₃, ZrO₂ (surface coatings: polymethylmethacrylate, methicone (methylhydrogenpolysiloxane), dimethicone, isopropyl titanium triisostearate, metal soaps such as magnesium stearate, perfluoroalcohol phosphate as C9-15 fluoroalcohol phosphate). Examples of UVA-absorbing agents include, but are not limited to, avobenzone (Parsol 1789), bisdisulizole disodium (Neo Heliopan AP), diethylamino hydroxybenzoyl hexyl benzoate (Uvinul A Plus), ecamsule (Mexoryl SX) and methyl anthranilate. Examples of UVB-blocking agents include, but are not limited to, 4-Aminobenzoic acid (PABA), cinoxate, ethylhexyl triazone (Uvinul T 150), homosalate, 4-Methylbenzylidene camphor (Parsol 5000), octyl methoxycinnamate (octinoxate), octyl salicylate (Octisalate), padimate 0 (Escalol 507), phenylbenzimidazole sulfonic acid (Ensulizole), polysilicone-15 (Parsol SLX) and trolamine salicylate. Examples of agents that block both UVA and UVB include, but are not limited to, bemotrizinol (Tinosorb S), benzophenones 1-12, ioxybenzone, drometrizole trisiloxane (Mexoryl XL), iscotrizinol (Uvasorb HEB), octocrylene, oxybenzone (Eusolex 4360), sulisobenzone, hybrid (chemical/physical): bisoctrizole (Tinosorb M), titanium dioxide and zinc oxide.

In addition, the compositions of the present technology may also include adjuvants and additives such as preservatives, organic solvents, browning agents, antioxidants, stabilizers, emollients, silicones, alpha-hydroxy acids, demulcents, anti-foaming agents, moisturizing agents, vitamins, fragrances, ionic or nonionic thickeners, surfactants, fillers, thickeners, sequestrants, polymers, propellants, alkalinizing or acidifying agents, opacifiers, fatty compounds (e.g., oil, wax, alcohols, esters, fatty acids), colorants, or mixtures thereof or any other ingredient that may be used for the production of compositions. The compositions of the present technology may be in the form of an aqueous solution, emulsions (oil in water or water in oil), a hydro alcoholic vehicle, a stick, an ointment, a gel, an aerosol (foams, sprays propellant pumps or the like).

In another embodiment of the present technology, the compounds disclosed herein may be formulated in other cosmetics and/or personal care products. The compounds of the present technology may be included into formulations used in the preparation of cosmetic products such as make-ups, for example in cream make-up, eye-care preparations, eye shadow preparations, mascara, eyeliner, eye creams or eye-fix creams; lip-care preparations, e.g., lipsticks, lip gloss, lip contour pencils, nail-care preparations, such as nail varnish, nail varnish removers, nail hardeners or cuticle removers. These products are formulated according to known methods in the art.

The compounds of the present technology may also be formulated into personal care products such as in skin-washing and cleansing preparations in the form of tablet-form or liquid soaps, detergents or washing pastes, bath preparations, e.g. liquid (foam baths, milks, shower preparations) or solid bath preparations, e.g. bath cubes and bath salts; skin-care preparations, e.g. skin emulsions, multi-emulsions or skin oils; cosmetic personal care preparations, e.g. facial make-up in the form of day creams or powder creams, face powder (loose or pressed), foot-care preparations, e.g. foot baths, foot powders, foot creams or foot balsams, special deodorants and antiperspirants or callus-removing preparations; light-protective preparations, such as sun milks, lotions, creams or oils, pre-tanning preparations or after-sun preparations; skin-tanning preparations, e.g. self-tanning creams; depigmenting preparations, e.g. preparations for bleaching the skin or skin-lightening preparations; insect-repellents, e.g. insect-repellent oils, lotions, sprays or sticks; deodorants, such as deodorant sprays, pump-action sprays, deodorant gels, sticks or roll-ons; antiperspirants, e.g. antiperspirant sticks, creams or roll-ons; preparations for cleansing and caring for blemished skin, e.g. synthetic detergents (solid or liquid), peeling or scrub preparations or peeling masks; hair-removal preparations in chemical form (depilation), e.g. hair-removing powders, liquid hair-removing preparations, cream- or paste-form hair-removing preparations, hair-removing preparations in gel form or aerosol foams; shaving preparations, e.g. shaving soap, foaming shaving creams, non-foaming shaving creams, foams and gels, preshave preparations for dry shaving, aftershaves or aftershave lotions; fragrance preparations, e.g. fragrances, perfume oils or perfume creams; cosmetic hair-treatment preparations, e.g. hair-washing preparations in the form of shampoos and conditioners, hair-care preparations, e.g. pretreatment preparations, hair tonics, styling creams, styling gels, pomades, hair rinses, treatment packs, intensive hair treatments, hair-structuring preparations, e.g. hair-waving preparations for permanent waves (hot wave, mild wave, cold wave), hair-straightening preparations, liquid hair-setting preparations, hair foams, hairsprays, bleaching preparations, e.g. hydrogen peroxide solutions, lightening shampoos, bleaching creams, bleaching powders, bleaching pastes or oils, temporary, semi-permanent or permanent hair colorants, preparations containing self-oxidizing dyes, or natural hair colorants, such as henna or chamomile. These products are formulated according to known methods in the art.

The compounds as defined herein may also be incorporated into formulation that may be used to protect hair (from humans or animals) against photochemical damage in order to prevent changes of color shades, discoloration or damage of a mechanical nature. In addition to the compounds defined herein, such cosmetic formulation may comprise various adjuvants used in this type of composition, such as surface-active agents, thickeners, polymers, softeners, preservatives, foam stabilizers, electrolytes, organic solvents, silicone derivatives, antigrease agents, dyes and/or pigments which color the composition itself or the hair, or other ingredients customarily used for hair care.

Ointments, pastes, creams and gels comprising the compounds of the present technology may include one or more carriers, such as, but not limited to, animal and vegetable fats, waxes, paraffins, starch, tragacanth, cellulose derivatives, polyethylene glycols, silicones, bentonites, silica, talc and zinc oxide or mixtures of these substances. Powders and sprays may include carriers, such as, but not limited to, lactose, talc, silica, aluminum hydroxide, calcium silicate and polyamide powder or mixtures of these substances, propellants, such as, but not limited to chlorofluorocarbons, propane/butane or dimethyl ether. Solutions and emulsions can include carriers, such as, but not limited to, solvents, solubility promoters and emulsifiers, e.g. water, ethanol, isopropanol, ethyl carbonate, ethyl acetate, benzyl alcohol, benzyl benzoate, propylene glycol, 1,3-butylglycol, oils, in particular cotton seed oil, peanut oil, wheatgerm oil, olive oil, castor oil and sesame oil, glycerol fatty acid ester, polyethylene glycols and fatty acid esters of sorbitan or mixtures of these substances. Soaps can include carriers, such as, but not limited to, alkali metal salts of fatty acids, salts of fatty acid mono esters, fatty acid protein hydrolysates, isethionates, lanolin, fatty alcohol, vegetable oils, plant extracts, glycerol, sugars or mixtures of these substances. Face and body oils can include carrier substances such as, but not limited to, synthetic oils, such as fatty acid esters, fatty alcohols, silicone oils, natural oils, such as vegetable oils and oily plant extracts, paraffin oils, lanolin oils or mixtures of these substances.

The compounds of the invention may also be formulated for topical administration. The term “topical” as used herein includes any route of administration that enables the compounds to line the skin or mucosal tissues. The compounds of the present invention may also be included into pharmaceutical formulations and/or compositions. These formulations and/or compositions are prepared according to known methods in the art. The formulations and the compositions of the present technology may also offer protection against ageing processes of the skin and against oxidative stress, against damage caused by free radicals, as are produced, for example, by solar irradiation, heat or other influences. The compounds of the present technology as well as the formulations and the compositions of the present technology may be used in the preparation and manufacture of medicaments for the prevention of damages to skin, such as, but not limited to, sunburn and sun-caused erythrema.

The cosmetic or pharmaceutical formulations and/or compositions according to the present technology may also comprise one or one more additional compounds such as but not limited to: alcohols, poly-alcohols, fatty alcohols, esters of fatty acids, natural or synthetic triglycerides including glyceryl esters and derivatives, pearlescent waxes, hydrocarbon oils, siliconces or siloxanes, fluorinated or perfluorinated oils, emulsifiers, surfactants, polymers, deodorizing active ingredients, antioxidants, hydrotropic agents, preservatives and bacteria-inhibiting agents, perfumes, colorants, preservatives, bactericides and bacteriostatic agents, perfumes, dyes, pigments, thickening agents, moisturizing agents, humectants, fats, oils, waxes, polymers, electrolytes, organic solvents, silicon derivatives, emollients, emulsifiers or emulsifying surfactants, surfactants, dispersing agents, antioxidants, anti-irritants and anti-inflammatory agents.

Examples of emulsifiers that may be included in the formulations and/or compositions of the present technology include, but are not limited to, cocoyl glucoside, cocoyl glucoside/cetearyl alcohol, cocoyl ethyl glucoside, disodium coco-glucoside citrate, lauryl glucoside, disodium coco-glucoside sulfosuccinate, lauroyl ethyl glucoside, myristoyl ethyl glucoside, octyl dimethicone ethoxy glucoside, oleoyl ethyl glucoside, sodium coco-glucoside tartrate, butylated PVP, cetyl alcohol, sodium acrylate/sodium acryloyldimethyltaurate copolymer, diethylhexyl napthalate, sorbitan oleate, sorbitan sesquioleate, sorbitan isostearate, sorbitan trioleate, polyglyceryl-3-diisostearate, polyglycerol ester of oleic/isostearic acid, polyglyceryl-6 hexaricinolate, polyglyceryl-4-oleate, polygylceryl-4 oleate/PEG-8 propylene glycol cocoate, oleamide DEA, sodium glyceryl oleate phosphate, hydrogenated vegetable glycerides phosphate, butylated PVP, cetyl alcohol, sodium acrylate/sodium acryloyldimethyltaurate copolymer, diethylhexyl napthalate, sodium stearoyl glutamate such as EUMULGIN®SG, sodium N-stearoyl L-glutamate, dioctyldodecyl stearoyl glutamate, TEA-cocoyl glutamate, TEA-lauryl glutamate, TEA-stearoyl glutamate, aluminum stearoyl glutamate, monosodium glutamate, disodium glutamate and any mixtures thereof.

### ii) Compositions Applied to Articles of Manufacture

In certain embodiments, the compounds of the present technology are used in the preparation of compositions for protecting non-biological materials against ultraviolet radiation and/or visible radiation. In some embodiments, the non-biological material is an article of manufacture. Examples of such articles of manufacture include, but are not limited to, windows and other glass, plexi-glass, transparent polymer, plastic or similar products, car windshields, solar panels, eyeglasses, sporting goods, textiles and fabrics. In other embodiments, the article of manufacture is a textile or fabric.

In some embodiments, the article of manufacture may be impregnated with or may be covered with a formulation and/or composition comprising the compounds disclosed herein. In other words, in certain implementations of these embodiments, the compounds of the present technology may be used in the preparation of coating compositions or compositions for integration with the article of manufacture. Such compositions prevent premature photodamage and photobleaching of the surface of these articles of manufacture and/or protect the articles of manufacture from exposure to radiation which causes ageing and weakening of their structure and strength.

In certain embodiments, the coating composition or the composition for integration with the article of manufacture further comprise one or mode additives. In some embodiments, the one or more additive is an organic solvent, an aqueous solvent, a binder, a surfactant, a wetting agent, a dispersing agent, or a cross-linking agent, or any combinations thereof.

In certain embodiments, the coating composition or the composition for integration with the article of manufacture can be prepared in an organic solvent alone, water alone or a mixture of the two depending on the compounds selected to be present in the composition.

Textile binders are necessary to form a matrix to entrap the compounds of the present technology and must be stable to outside forces, such as washing or rubbing, that would tend to dislodge compounds of the present technology from the textile substrate. Binders suitable for the compositions of the present technology include, but are not limited to, binders based on styrene butadiene, styrene acrylate, vinyl acetate-acrylate co-polymer, urethane, acrylonitrile, and Melamine.

In certain embodiments, the binder is rewettable. In other embodiments, the composition may further comprise a surfactant to improve rewettability of the binder, if the binder selected has poor rewettability. The rewettability characteristics of the binder are important factors in the development of the compositions of the present technology as poor rewetting characteristics promote premature polymerization which can lead to binder build up on equipment, pads and dry cans, which in turn leads to contamination of the shade and poor quality.

In certain embodiments, the pH of the coating composition comprising the compound of the present technology is at least about 7, or between about between about 7 and about 14, or about 7.0. The pH of the coating composition is generally maintained at about 7.0 or higher to minimize flocculation in dye baths used to coat textiles. As used herein, the term “flocculation” refers to a process by which colloidal particles come out of suspension to sediment under the form of floc or flake.

In further embodiments, the compositions of the present technology comprise a cross-linking agent. In certain implementation of these embodiments, the cross-linking agent is a resin. The resin links the binder polymer to itself to increase its durability and enhances wet fastness. The choice of crosslinking agent generally takes place according to the nature of the binder selected. Examples of crosslinking agents suitable for the compositions of the present technology include, but are not limited to disiocyanate, aliphatic polyisocyanate, blocked isocyanate, blocked isocyanate crosslinking agent, and Desmodur.

The compositions of the present technology may be prepared by mixing (or mechanically agitating) of the compounds disclosed herein and any additional optional components, to form a homogenous mixture. This may be accomplished by any convenient mixing method known in the art exemplified by a spatula, mechanical stirrers, in-line mixing systems containing baffles and/or blades, powered in-line mixers, homogenizers, a drum roller, a three-roll mill, a sigma blade mixer, a bread dough mixer, and a two-roll mill.

In some embodiments, the coating composition may be a coating, paint, sealant, adhesive, dye, varnish, stain, coloring composition, flame retardant, adhesives, lacquers and the like.

The coating compositions of the present technology may be coated on any textile substrate amenable to use with such coating compositions and methods. Suitable textile substrates for use with the present invention include textiles having natural, synthetic, cellulose-based, or non-cellulose-based fibers or any combination thereof. Exemplary textile substrates include, but are not limited to, textiles having hydroxy group-containing fibers such as natural or regenerated cellulosic fibers (cotton, rayon, and the like); nitrogen group-containing fibers such as polyacrylonitrile; natural or synthetic polyamides (including wool, silk, or nylon); and/or fibers having acid-modified polyester and polyamide groups. The substrates may be additionally pre-treated or after-treated with resins or other substances compatible with the coating compositions of the present technology, and may be finished or unfinished. The textile substrate may also be sized prior to application of the present coating compositions. Alternatively, the present coating compositions may be incorporated into an external sizing process, so that sizing and coating is conducted in a single step.

The fibers of the textile substrate may be in any suitable form including for example loose yarns, or fabrics. Fabrics are a convenient and preferred form. The fibers may be blended with other fibers that are susceptible to treatment with the coating composition of the present technology, or with fibers that may prove less susceptible to such treatment. The process may also be used with leather, vinyl and other natural or synthetic materials. Additional exemplary substrates for use in the present technology include polyester films such as “MYLAR” flexible film, polysulfones, cellulose triacetates, and the like. Coated transparent films are also contemplated.

In certain embodiments, the compounds or the composition for integration in the non-biological material may be integrated into the substrate of the non-biological material which constitutes the base formulation for the manufacture of said non-biological material. For example, the compounds of the present invention may be incorporated into a substrate which constitutes the base formulation of liquid coatings or powder coatings, or the base resin of an article to be fabricated using conventional plastic compounding, molding or extrusion processes. The substrates into which the compounds of the present technology may be incorporated include a wide variety of resin and plastic materials, for example, polyurethane, polyolefins, polyvinylaromatics, acrylics, polycarbonates, polyesters, polyamides, polyimides, polyarylates, polysulfones, polybutenes, polypropenes, epoxies, and polyvinylhalide resins and generally any resin known to be susceptible to degradation being exposed to ultraviolet light radiation. Naturally, the choice of compound to be incorporated into such substrate must be made such that, at the temperatures for processing the paints, coatings, finishes or thermoplastic articles, the compounds of the present technology do not undergo substantial degradation or cross reaction with any other ingredients of the formulation. Representative, but non-limiting, examples of specific polymeric resin materials include polyurethane resins, such as thermoplastic polyurethane resins, polyolefin resins such as polyethylene and polypropylene and the like; polyvinylaromatic resins such as polystyrene and copolymers and terpolymers therefor, such as poly(styrene-acrylonitrite) and poly(styrene-butadieneacrylonitrile) and the like; acrylic resins such as poly(acrylic acid), poly(methacrylic acid), poly(methyl acrylate), poly(methyl methacrylate) and the like; polycarbonate resins such as those obtained either by the phosgenation of dihydroxy aliphatic and aromatic monomers such as ethylene glycol, propylene glycol, bisphenol A (i.e., 4,4′-isopropylidene diphenol) and the like, or by the base catalyzed transesterification of bisphenol A with diphenylcarbonate to produce bisphenol A polycarbonate; polyester resins such as poly(ethylene terephthalate), poly(butylene terephthalate) and the like; polyamide resins such as nylon-6, nylon-6.6 and the like; epoxy resins such as poly(epichlorohydrin/bisphenol A) and the like, and esters thereof such as the epoxy resin esters prepared by the esterification of poly(epichlorohydrin/bisphenol A) with a fatty acid, rosin acid, tall oil acid or mixtures thereof, and phenolic resins such as those prepared by reaction of formaldehyde with phenol, resorcinol, cresol, xylenol, p-tert-butylphenol and the like. In certain embodiments, the amount of compound integrated into the substrate of the non-biological material is from about 0.1% to about 5%, about 0.2% to about 4%, or about 0.5% to about 2% of the total weight of substrate mix to be used for the article of manufacture.

The textiles or fabrics that have been applied or integrated with the compounds of the technology are herein referred to as “treated textiles” and “treated fabrics”. Resistance of the treated textiles or treated fabrics to exposure to UV radiations maybe assessed by determining such properties of the treated textiles and treated fabrics as, but not limited to, color fastness, breaking strength, and/or tensile strength by the strip method following UV exposure. The techniques for determining these properties of a treated textile or a treated fabric are well known in the art.

### Methods

From another aspect, the present technology provides methods for protecting a surface of a biological or non-biological material against UV radiation and/or visible radiation comprising applying to the surface of said biological or non-biological material a composition comprising any one or more of the compounds of the present technology or any combination thereof.

In certain embodiments, the present technology provides for methods of preventing and/or treating biological materials from harmful effects of solar radiation, including UV and/or visible radiation. In particular, the present technology provides a method for preventing harmful effects of solar radiation on a subject such as a human. Examples of harmful effects of solar radiation on biological material include but are not limited to, sunburn, inflammation, melanoma, malignant melanoma, DNA damage, eye damages, erythema and local or systemic immuno-suppression. In one implementation of this embodiment, the method is for preventing the harmful effects of UV and/or visible radiations on a subject such as a human and includes the steps of applying a formulation and/or a composition comprising one or more of the compounds of the present technology onto the skin of the human subject. The method may also be used to protect skin of animal subjects. In some embodiments, the application is a topical application.

In other embodiments, the present technology provides for methods of preventing non-biological material from harmful effect of solar radiation. Examples of harmful effects of solar radiation, including UV and/or visible radiation on non-biological material include, but are not limited to, premature photodamage and photobleaching and weakening of the structure and strength. In one implementation of this embodiment, the method is for preventing the harmful effect of UV and/or visible radiation on textiles or fabrics and includes the steps of applying a formulation and/or a composition comprising one or more of the compounds of the technology onto the surface of the non-biological material. In further implementations of these embodiments, the composition may be applied to the surface of non-biological material by spraying, padding or dyeing. In yet further embodiments, the method is for preventing the harmful effect of UV and/or visible radiation on an article of manufactures and includes the steps of extruding one or more of the compounds of the present technology with a substrate of said article of manufacture.

### EXAMPLES

The examples below are given so as to illustrate the practice of various embodiments of the present disclosure. They are not intended to limit or define the entire scope of this disclosure. It should be appreciated that the disclosure is not limited to the particular embodiments described and illustrated herein but includes all modifications and variations falling within the scope of the disclosure as defined in the appended embodiments. A flow chemical process for manufacturing compounds which absorb UV radiation and/or visible radiation and protect biological materials as well as non-biological materials from damaging UV radiation and/or visible radiation are described hereinafter in the Examples.

### Example 1: Synthesis of the Compound of Formula 1

A 1 L round bottom flask was charged with 35 g of dimedone (formula 2), 36.6 g of glycine ethyl ester. HCl, 22.1 ml of pyridine and 500 mL of acetonitrile and a magnetic stir bar. The suspension was heated (oil bath, 95° C., solution not quite at reflux) and stirred overnight. The solvent was removed under vacuum. The resulting yellow oil was diluted in CH₂Cl₂ and extracted twice with water, brine and dried with MgSO₄. The crude was recrystallized with 200 mL hot acetonitrile, crystallization occurred upon cooling to room temperature. The crystalline solid was collected on a frit and washed with 100 mL of cold acetonitrile and dried in air. Yield 29 g (51.6%).

### Example 2: Synthesis of Compound of Formula 3

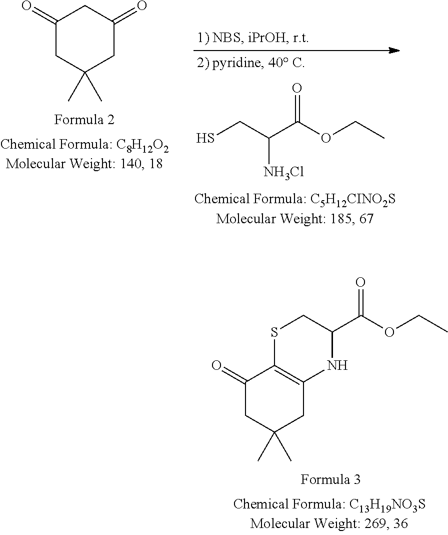

A 2 L round bottom flask was charged with 62.5 g of dimedone (formula 2), 500 mL of 2-propanol (IPA) and a magnetic stir bar. NBS (92.5 g) was added portion wise over a 10-minute period. The mixture was heterogenous and yielded a white slurry. The mixture was stirred for 20 minutes. Pyridine (80 mL) were added to the mixture and L-cysteine ethyl ester hydrochloride (100 g) was added portion wise keeping internal temperature below 30° C. The mixture turned red and homogeneous and was heated at 40° C. for 2 hours. After 2 hours at 40° C., the red solution was concentrated to about 250 mL volume. Crystals started to precipitate, the slurry was poured into 1 L of distilled water and stirred for 5 minutes before being filtered on a frit and washed with 250 mL of distilled water. The crude was recrystallized with 150 mL IPA, crystallization occurred upon cooling to room temperature, further crystallization occurs when the mixture was placed in the freeze overnight. The crystalline solid was collected on a frit and washed with 100 mL of IPA and dried in air. Yield 65.6 g (54.6%).

### Example 3: Synthesis of Compound Formula 4

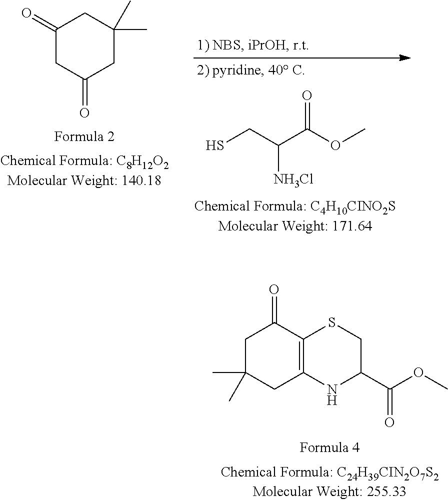

A 1 L round bottom flask was charged with 32 g of dimedone (formula 2), 90 mL of 2-propanol (IPA) and a magnetic stir bar. NBS (48 g) was added portion wise over a 10-minute period. The mixture was heterogenous and yielded a white slurry. The mixture was stirred for 20 minutes. Pyridine (39 mL) were added to the mixture and L-cysteine methyl ester hydrochloride (53 g) was added portion wise keeping internal temperature below 30° C. The mixture turned red and homogeneous and was heated at 40° C. for 2 hours. After 2 hours at 40° C., the red solution was concentrated to about 250 mL volume. Crystals started to precipitate, the slurry was poured into 1 L of distilled water and stirred for 5 minutes before being filtered on a frit and washed with 250 mL of distilled water. The crude was recrystallized with 150 mL IPA, crystallization occurred upon cooling to room temperature, further crystallization occurred when the mixture was placed in the freeze overnight. The crystalline solid was collected on a frit and washed with 100 mL of IPA and dried in air. Yield 26.5 g (45%).

### Example 4: Synthesis of Compound of Formula 5

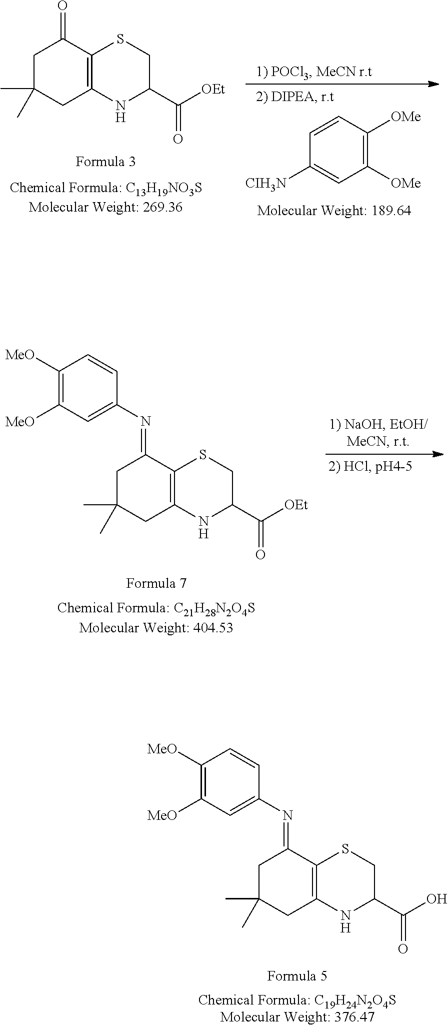

Under inert atmosphere, POCl₃ (1.9 ml) was added to a solution of a compound of formula 3 (5 g) in dry AcCN (70 ml). The solution was stirred at room temperature for 1 h30. First 3,4-Dimethoxyaniline. HCl (4.2 gr) as solid and then DiPEA (6.1 ml) by syringe was added to solution and stirred at RT for an hour. After confirming complete conversion of the starting material AcCN was removed under vacuum and the resulting oil was diluted in CH₂Cl₂(200 ml), extracted with water and brine, and dried with MgSO₄. The solvent was evaporated, and the resulting impure compound (formula 7) powder was purified by column chromatography on 50 gr silica. Mobile phase: DCM/MeOH 100/0, 99/land finally 95/5. Different fractions were obtained, dried separately, and checked by TLC. First fractions were brown and impure. Middle fractions were very pure. The combined solids were collected to yield the compound of formula 7 (4 gr, 53%) as a yellow solid. The compound of formula 7 (7 gr) was then suspended in AcCN (20 ml) and 10 ml of a NaOH solution (10%) in ethanol was added. The reaction mixture was stirred at room temperature for an hour. The suspension was acidified with concentrated HCl to pH 5.3 and filtered. The solvent was evaporated under vacuum to isolate the compound of formula 5 (3.5 g, 55%) as an orange-yellow solid.

### Example 5: Synthesis of Compound of Formula 8

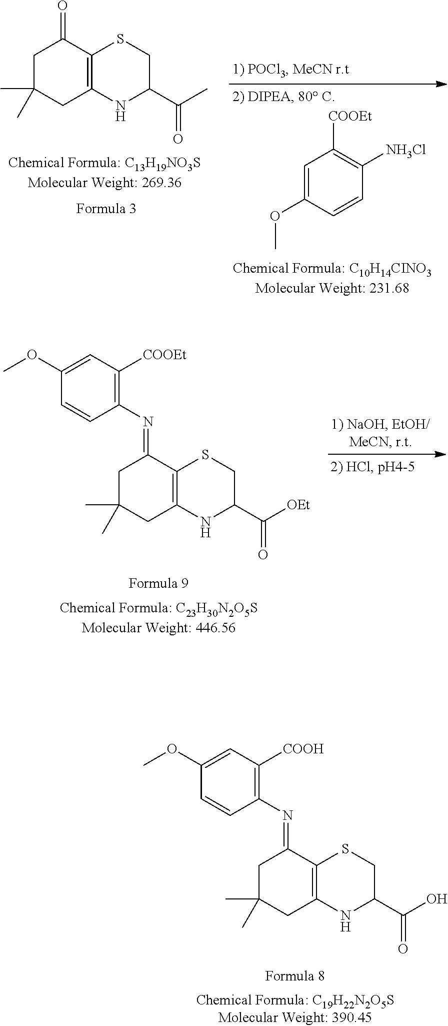

Under inert atmosphere, POCl₃ (2.28 ml) was added to a solution of a compound of formula 6 (6 g) in dry AcCN (100 ml). The solution was stirred at room temperature for 1 h30. First Ethyl-2-amino-5 methoxybenzoate. HCl (6 gr) as solid and then DiPEA (9.33 ml) by syringe was added to solution and stirred at 80° C. for 2 hour. After confirming complete conversion of the starting material AcCN was removed under vacuum and the resulting oil was diluted in CH₂C12(200 ml), extracted with water and brine, and dried with MgSO4. The solvent was evaporated, and the resulting impure compound (formula 9) powder was purified by column chromatography on 50 gr silica. Mobile phase: DCM/Ethyl acetate 50/50, 70/30, 90/10 and 100/0. After washing column with a lot of DCM, 1% methanol was added. The different fractions were obtained, dried separately, and checked by TLC. First fractions were brown and impure. The combined solids were collected to yield the compound of formula 9 (4.96 gr, 50%) as a pale-orange solid. The compound of formula 9 (2.5 gr) was then suspended in AcCN (20 ml) and 10 ml of a NaOH solution (10%) in ethanol was added. The reaction mixture was stirred at room temperature for an hour. The suspension was acidified with concentrated HCl to pH 5.3 and filtered. The solvent was evaporated under vacuum and again dissolved in a minimum amount of MeOH and 8 times more CH₂Cl₂ was added. Precipitation appeared in the flask during evaporation which was filtered and dried to isolate the compound of formula 8 (1.6 gr, 76%) as a pale-orange solid.

### Example 6: Synthesis of Compound of Formula 10

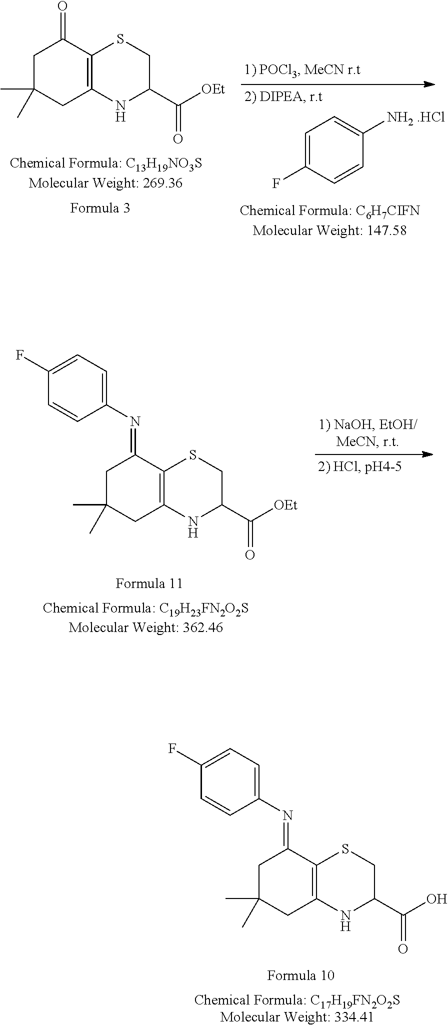

Under inert atmosphere, POCl₃ (1.9 ml) was added to a solution of a compound of formula 3 (5 g) in dry AcCN (70 ml). The solution was stirred at room temperature for 1 h30. First Fluoroaniline. HCl (3 gr) as solid and then DiPEA (7.12 ml) by syringe was added to solution and stirred at RT for 4 hour. After confirming complete conversion of the starting material AcCN was removed under vacuum and the resulting oil was diluted in CH₂Cl₂ (200 ml), extracted with water and brine, and dried with MgSO₄. The solvent was evaporated, and the resulting impure compound (formula 11) powder was purified by column chromatography on 50 gr silica. Mobile phase: DCM/MeOH 100/0, 99/land finally 95/5. The different fractions were obtained, dried separately, and checked by TLC. The first fractions were brown and impure. The middle and final fractions were pure. The combined solids were collected to yield the compound of formula 11 (4 gr, 44%) as a yellow solid. The compound of formula 11(1.3 gr) was then suspended in AcCN (20 ml) and 10 ml of a NaOH solution (10%) in ethanol was added. The reaction mixture was stirred at room temperature for an hour. The suspension was acidified with concentrated HCl to pH 5.3 and filtered. The solvent was evaporated under vacuum and again dissolved in minimum amount of MeOH and 10 times more CH₂Cl₂ was added. Precipitation appeared in flask during evaporation which was filtered and dried to isolate the compound of formula 10 (550 mg, 46%) as a yellow solid.

### Example 7: Synthesis of Compound of Formula 12

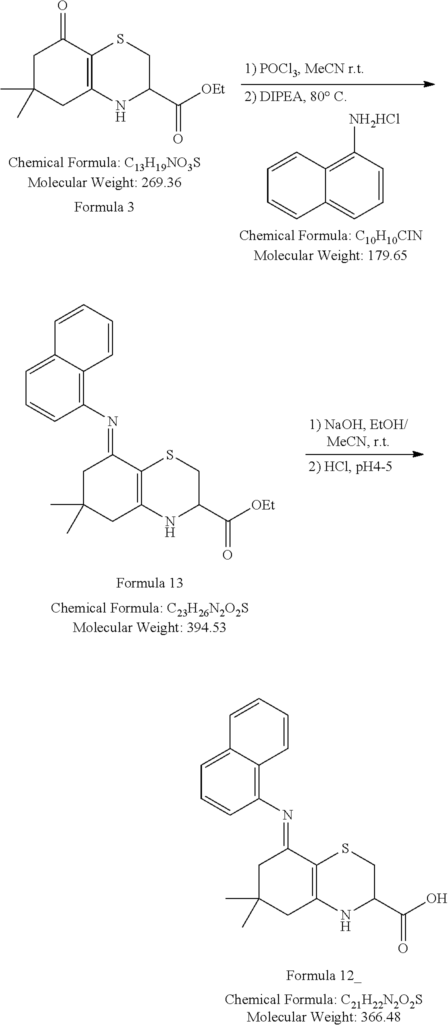

Under inert atmosphere, POCl₃ (3.81 ml) was added to a solution of a compound of formula 3 (10 g) in dry AcCN (70 ml). The solution was stirred at room temperature for 1 h30. First 1-Naphtylamine. HCl (8 gr) as solid and then DiPEA (15.54 ml) by syringe was added to solution and stirred at 80° C. for an hour. After confirming complete conversion of the starting material, AcCN was removed under vacuum and the resulting oil was diluted in CH₂Cl₂ (200 ml), extracted with water and brine, and dried with MgSO4. The solvent was evaporated, and the resulting impure compound (formula 13) powder was purified by column chromatography on 50 gr silica. Mobile phase: DCM/MeOH 100/0, 99/land finally 98/2. The different fractions were obtained, dried separately, and checked by TLC. The first two fractions were brown and impure. The middle fractions were very pure. The combined solids were collected to yield the compound of formula 13 (13 g, 90%) as a yellow solid. The compound of formula 13 (6.5 gr) was then suspended in AcCN (20 ml) and 15 ml of a NaOH solution (10%) in ethanol was added. The reaction mixture was stirred at room temperature for an hour. The suspension was acidified with concentrated HCl to pH 5.3 and filtered. The solvent was evaporated under vacuum and again dissolved in a minimum amount of MeOH and 10 times more CH₂Cl₂ was added. Precipitation appeared in the flask during evaporation which filtered and dried to isolate the compound of formula 12 (2.7 g, 44%) as a yellow solid.

### Example 8: Synthesis of Compound Formula 14

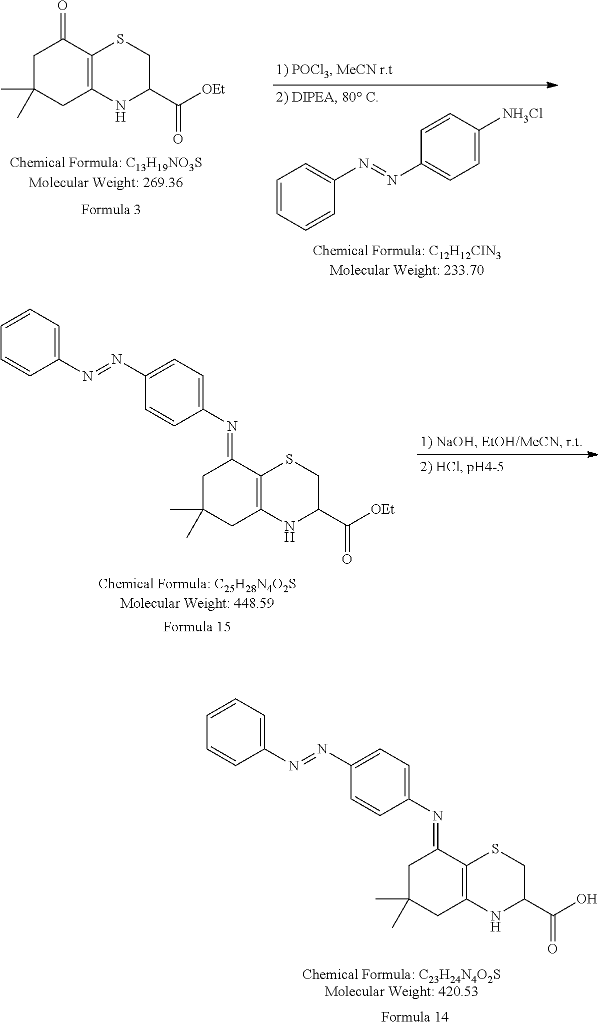

Under inert atmosphere, POCl₃ (1.52 ml) was added to a solution of a compound of formula 3 (4 g) in dry AcCN (70 ml). The solution was stirred at room temperature for 1 h30. First 4-Aminoazobenzoate. HCl (3.46 gr) as solid and then DiPEA (5.18) by syringe was added to solution and stirred at RT overnight. After confirming complete conversion of the starting material, AcCN was removed under vacuum and the resulting oil was diluted in CH₂Cl₂ (200 ml), extracted with water and brine, and dried with MgSO₄. The solvent was evaporated, and the resulting impure compound (formula 15) powder was purified by column chromatography on 80 gr silica. Mobile phase: DCM/MeOH 100/0, 99/land finally 95/5. The different fractions were obtained, dried separately, and checked by TLC. The first fractions were brown and impure. The combined solids were collected to yield the compound of formula 15 (5.7 g, 86%) as a dark-orange solid. The compound of formula 15 (1 gr) was then suspended in AcCN (20 ml) and 5 ml of a NaOH solution (10%) in ethanol was added. The reaction mixture was stirred at room temperature for an hour. The suspension was acidified with concentrated HCl to pH 5.3 and filtered. The solvent was evaporated under vacuum and purified by column chromatography on 50 gr silica. Mobile phase: DCM/MeOH 100/0. After washing with 1 L of DCM, methanol 5% was added and after evaporation of solvent, the compound of formula 14 (500 mgr, 53% yield) was obtained.

### Example 9: Synthesis of Compound for Formula 16

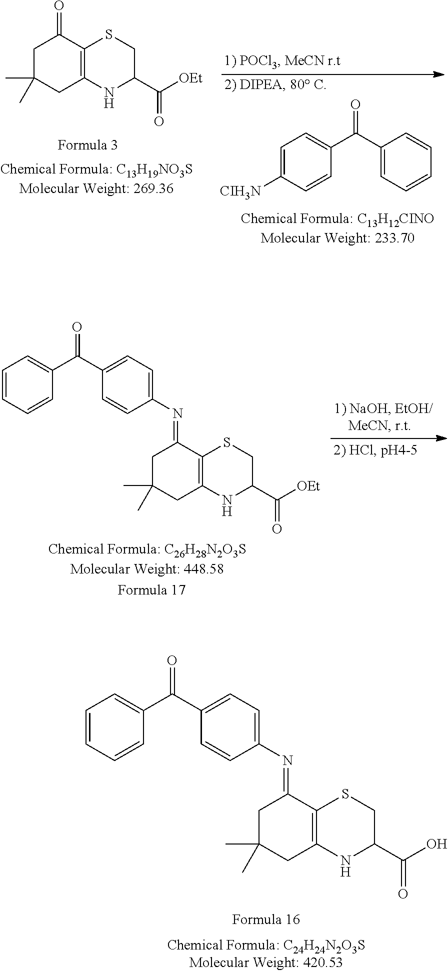

Under inert atmosphere, POCl₃ (3.4 ml) was added to a solution of a compound of formula 3 (8 g) in dry AcCN (70 ml). The solution was stirred at room temperature for 1 h30. First 4-amino-benzophenone. HCl (6.9 gr) as solid and then DiPEA (10.36 ml) by syringe was added to solution and stirred at 80° C. for 2 hours. After confirming complete conversion of the starting material, AcCN was removed under vacuum and the resulting oil was diluted in CH₂Cl₂ (300 ml), extracted with water and brine, and dried with MgSO4. The solvent was evaporated, and the resulting impure compound (formula 17) powder was purified by crystallization in acetonitrile/THF, 70/30 solvent and stirring overnight. The combined solids were collected to yield the compound of formula 17 (10.8 g, 83%) as a bright yellow solid. The compound of formula 17 (1 gr) was then suspended in AcCN (10 ml) and 5 ml of a NaOH solution (10%) in ethanol was added. The reaction mixture was stirred at room temperature for an hour. The suspension was acidified with concentrated HCl to pH 5.3 and filtered. The solvent was evaporated under vacuum to isolate the compound of formula 16 (850 mg, 90%) as a yellow solid.

### Example 10: Synthesis of Compound of Formula 18

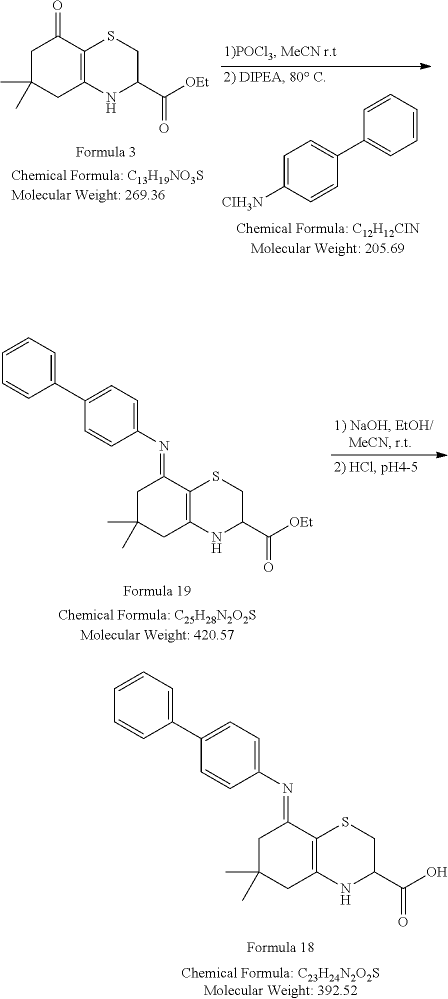

Under inert atmosphere, POCl₃ (2.28 ml) was added to a solution of a compound of formula 3 (6 g) in dry AcCN (70 ml). The solution was stirred at room temperature for 1 h30. First 4-amino-biphenyl. HCl (4.5 gr) as solid and then DiPEA (7.77 ml) by syringe was added to the solution and stirred at 80° C. for 2 hours. After confirming complete conversion of the starting material. AcCN was removed under vacuum and the resulting oil was diluted in CH₂Cl₂ (300 ml), extracted with water and brine, and dried with MgSO₄. The solvent was evaporated, and the resulting impure compound (formula 19) powder was dissolved in a minimum amount of ACN/THF/MTBE: 10/10/40 and stirred over weekend. Yellow crystals formed. The combined solids were collected to yield the compound of formula 19 (6.4 g, 64%) as a yellow solid. The compound of formula 19 (1 gr) was then suspended in AcCN (10 ml) and 5 ml of a NaOH solution (10%) in ethanol was added. The reaction mixture was stirred at room temperature for an hour. The suspension was acidified with concentrated HCl to pH 5.3 and filtered. The solvent was evaporated under vacuum to isolate the compound of formula 18 (600 mg, 64%) as a yellow solid.

### Example 11: Synthesis of Compound for Formula 20

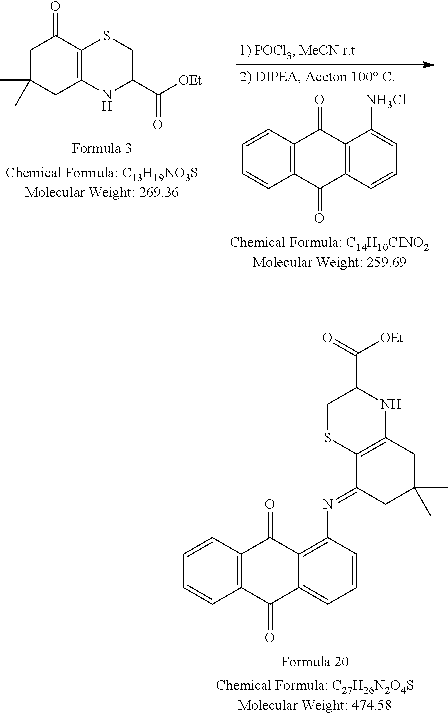

Under inert atmosphere, POCl₃ (0.76 ml) was added to a solution of a compound of formula 3 (2 g) in dry AcCN (70 ml). The solution was stirred at room temperature for 1 h30. First 4-Amino-anthraquinone (2.3 gr) as solid, DiPEA (3.1 ml) by syringe and then 50 ml acetone was added and stirred at 80° C. for 4 hours. After confirming complete conversion of the starting material, AcCN was removed under vacuum and the resulting oil was diluted in CH₂Cl₂ (300 ml), extracted with water and brine, and dried with MgSO₄. The solvent was evaporated, and the resulting impure compound of formula 20 was purified by column chromatography on 50 gr silica. Mobile phase: DCM/MeOH 100/0, 99/1 and finally 98/2. The different fractions were obtained, dried separately, and checked by TLC. The first fractions were brown and impure by non-reacted amine. The combined solids were collected to yield the compound of formula (1.6 g, 42%) as a red-orange solid.

### Example 12: Synthesis of Compound of Formula 21

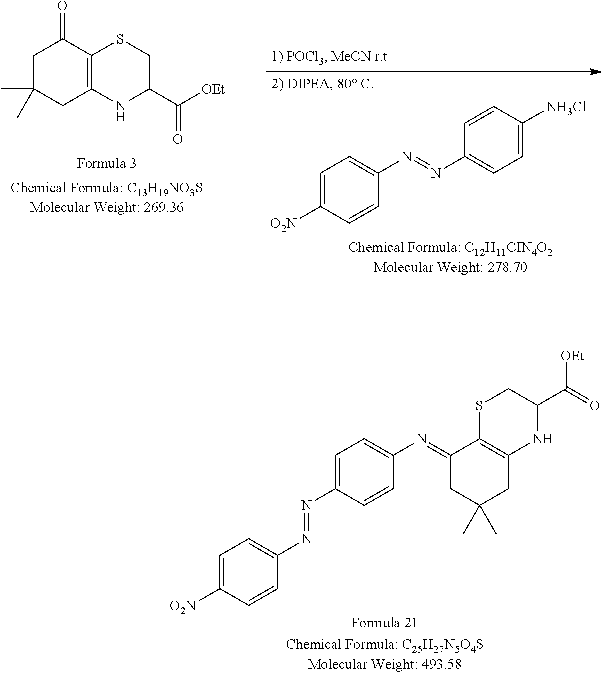

Under inert atmosphere, POCl₃ (0.57 ml) was added to a solution of a compound of formula 3 (1.5 g) in dry AcCN (70 ml). The solution was stirred at room temperature for 1 h30. First 4-(4-Nitrophenylazo) aniline. HCl (1.5 gr) as solid and then DiPEA (1.94 ml) by syringe was added to solution and stirred at 80° C. for 3 hours. After confirming complete conversion of the starting material, AcCN was removed under vacuum and the resulting oil was diluted in CH₂C12(300 ml), extracted with water and brine, and dried with MgSO₄. The solvent was evaporated, and the resulting impure compound of formula 21 was purified by column chromatography on 50 gr silica. Mobile phase: DCM/MeOH 100/0, 99/land finally 97/3. The different fractions were obtained, dried separately, and checked by TLC. The first fractions were impure by non-reacted amine. The combined solids were collected to yield the compound of formula 21 (2.3 g, 85%) as an orange solid.

### Example 13: Synthesis of Compound of Formula 22

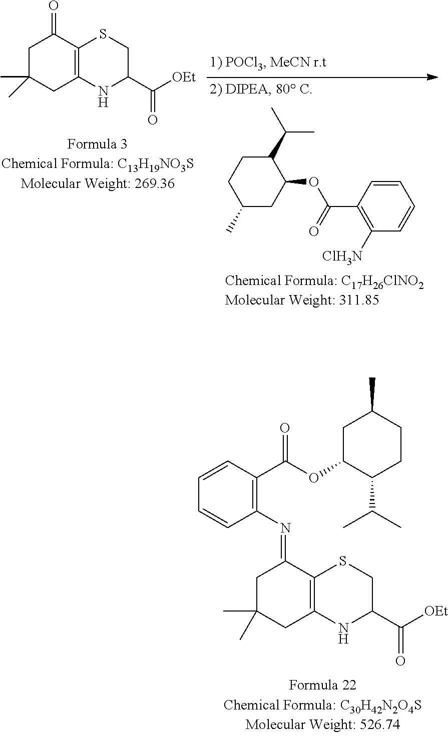

Under inert atmosphere, POCl₃ (2.66 ml) was added to a solution of a compound of formula 3 (7 g) in dry AcCN (70 ml). The solution was stirred at room temperature for 1 h30. First Menthy anthranilate. HCl (6.95 gr) as solid and then DiPEA (9 ml) by syringe was added to the solution and stirred at 80° C. for 2 hours. After confirming complete conversion of the starting material, AcCN was removed under vacuum and the resulting oil was diluted in CH₂Cl₂ (300 ml), extracted with water and brine, and dried with MgSO₄. The solvent was evaporated, and the resulting impure compound of formula 22 was purified by column chromatography on 50 gr silica. Mobile phase: DCM/MeOH 100/0, 99/land finally 97/3. The different fractions were obtained, dried separately, and checked by TLC. The first fractions were impure by non-reacted amine. The combined solids were collected to yield the compound of formula 22 (8.6 g, 63%) as a yellow-orange solid.

### Example 14: Synthesis of Compound of Formula 23

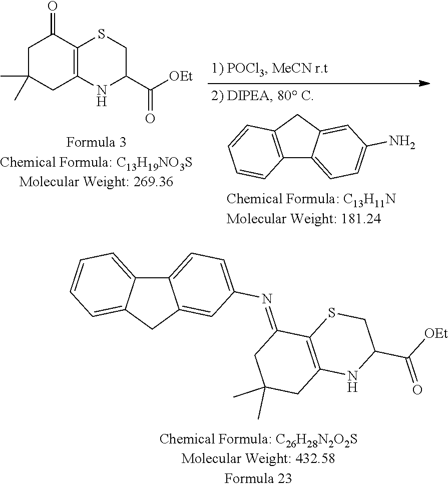 [SMILES: CCOC(=O)C1CSC2=C(CC(C)(C)CC2=O)N1 ; Nc1ccc2c(c1)Cc1ccccc1-2 ; CCOC(=O)C1CSC2=C(CC(C)(C)C/C2=N\c2ccc3c(c2)Cc2ccccc2-3)N1]

Under inert atmosphere, POCl₃ (1.9 ml) was added to a solution of a compound of formula 3 (5 g) in dry AcCN (70 ml). The solution was stirred at room temperature for 1 h30. First 4-aminoflourene (3.2 gr) as solid and then DiPEA (6.48 ml) by syringe was added to the solution and stirred at 80° C. for 2 hours. After confirming complete conversion of the starting material, AcCN was removed under vacuum and the resulting oil was diluted in CH₂Cl₂ (300 ml), extracted with water and brine, and dried with MgSO₄. The solvent was evaporated, and the resulting impure compound of formula 23 was purified by column chromatography on 50 gr silica. Mobile phase: DCM/MeOH 100/0, 99/land finally 97/3. The different fractions were obtained, dried separately, and checked by TLC. The first fractions were impure by non-reacted amine. The combined solids were collected to yield compound of formula 23 (4 g, 50%) as a yellow-orange solid.

### Example 15—Synthesis of Compound for Formula 24

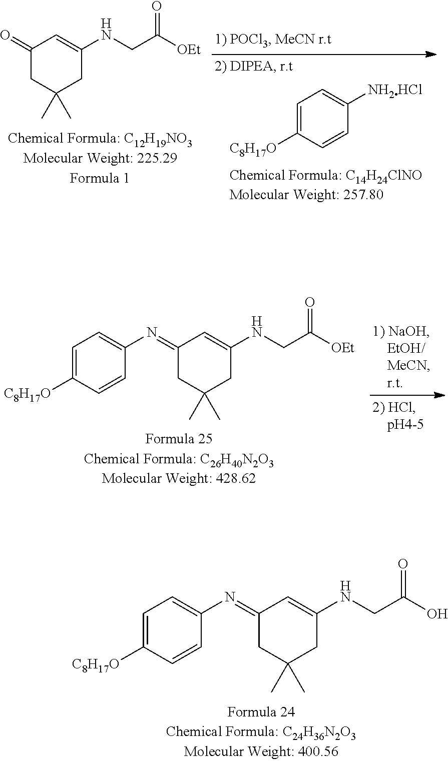

Under inert atmosphere, POCl₃ (2.73 ml) was added to a solution of a compound of formula 1 (6 g) in dry ACCN (70 ml). The solution was stirred at room temperature for 1 h30. First 4-(Octyloxy) aniline. HCl (6.5 gr) as solid and then DiPEA (9.29 ml) by syringe was added to solution and stirred at RT for an hour and at 50° C. for an hour. After confirming complete conversion of the starting material, ACCN was removed under vacuum and the resulting orange oil was diluted in CH₂Cl₂ (100 ml), extracted with water and brine, and dried with MgSO₄. The solvent was evaporated and addition of acetone (70 ml)/Dichloromethane (10 ml), to the resulting orange oil lead the formation of a beige solid. The product was isolated by filtration and the filtrate was evaporated. The combined solids were collected to yield the compound of formula 25 (7.4 g, 64%) as a beige solid. The compound of formula 25 (1.8 g) was suspended in ACCN (10 ml) and a solution of NaOH (15 ml, 0.83 g/l) in ethanol was added. The reaction mixture was stirred at room temperature for an hour. The suspension was acidified with concentrated HCl to pH 5.3 and filtered. The solvent was partially evaporated under vacuum before adding acetone which induced crystallization. The suspension was filtered, and the residue was rinsed with cold acetone to isolate the compound of formula 24 (1.2 g, 75%) as a beige solid.

### Example 16: Synthesis of Compound of Formula 26

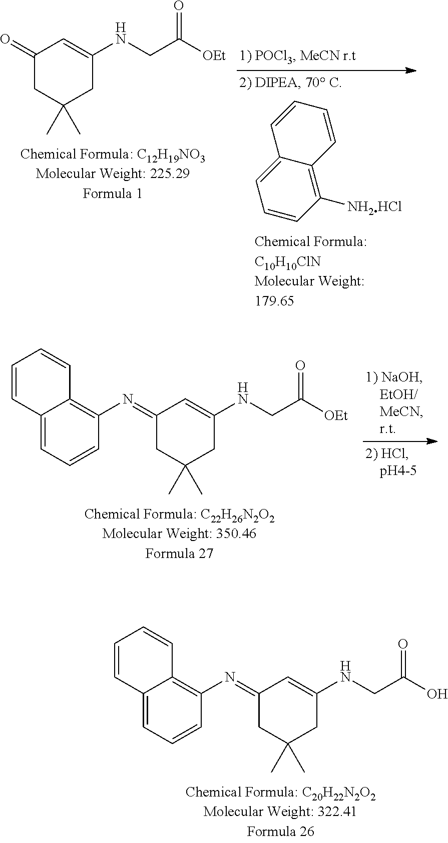

Under inert atmosphere, POCl₃ (2.28 ml) was added to a solution of a compound of formula 1 (5 g) in dry ACCN (70 ml). The solution was stirred at room temperature for 1 h30. First 1-Naphtylamine. HCl (4.3 gr) as solid and then DiPEA (8.7 ml) by syringe was added to the solution and stirred at RT for an hour and at 70° C. for an hour. After confirming complete conversion of the starting material, ACCN was removed under vacuum and the resulting orange oil was diluted in CH₂Cl₂ (100 ml), extracted with water and brine, and dried with MgSO₄. The solvent was evaporated, and the resulting 7 gr impure compound (formula 27) powder was purified by column chromatography on 50 gr silica. Mobile phase: DCM/MeOH 100/0, 98/2, 97/3 and finally 95/5. The different fractions were obtained, dried separately, and checked by TLC. The first two fractions were brown and impure. Middle fractions were very pure. The combined solids were collected to yield the compound of formula 27 (5 g, 64%) as a beige-yellow solid. The compound of formula 27 (1 gr) was then suspended in ACCN (10 ml) and 15 ml of a NaOH solution (10%) in ethanol was added. The reaction mixture was stirred at room temperature for an hour. The suspension was acidified with concentrated HCl to pH 5.3 and filtered. The solvent was evaporated under vacuum and again dissolved in minimum amount of MeOH and 10 times more THF was added. Precipitation appeared in flask during evaporation which was filtered and dried to isolate the compound of formula 26 (0.7 g, 76%) as a beige-yellow solid.

### Example 17: Synthesis of Compound of Formula 28

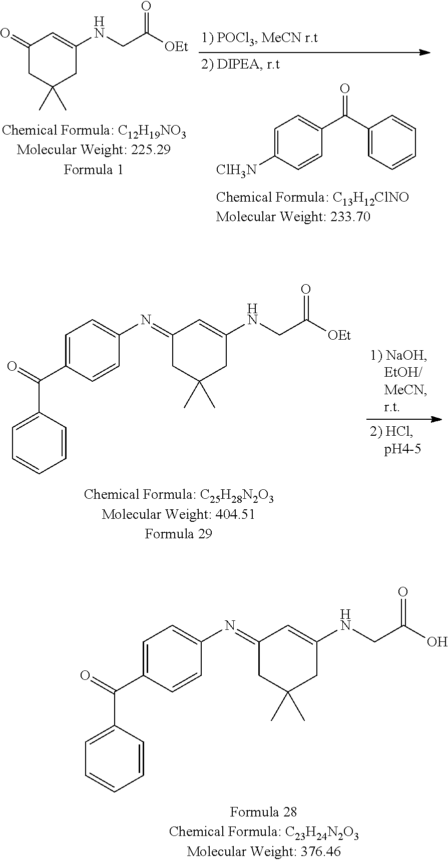

Under inert atmosphere, POCl₃ (5.46 ml) was added to a solution of a compound of formula 1 (12 g) in dry ACCN (100 ml). The solution was stirred at room temperature for 1 h30. First 4-Aminobenzophenon (12.4 gr) as solid and then DiPEA (18.59 ml) by syringe was added to the solution and stirred at RT for two hours. The solution became cloudy after 10 min. The reaction mixture was filtered and washed with cold acetonitrile and powder dried over frit. Pale yellow Crystals were collected to yield the compound of formula 29 (15 g, 70%). The compound of formula 29 (3 gr) was then suspended in ACCN (20 ml) and 27 ml of a NaOH solution (10%) in ethanol was added. The reaction mixture was stirred at room temperature for an hour. The suspension was acidified with concentrated HCl to pH 5.3 and filtered. The solvent was evaporated under vacuum to isolate the compound of formula 28 (2.6 g, 94%) as a beige-yellow solid.

### Example 18: Synthesis of Compound of Formula 30

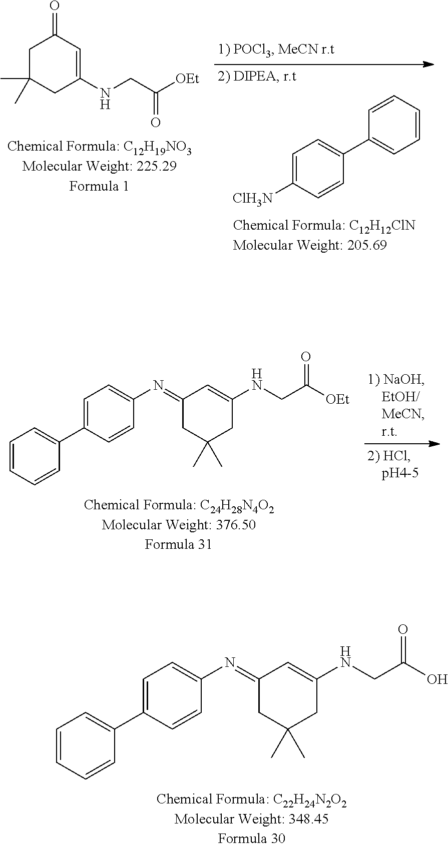

Under inert atmosphere, POCl₃ (5.46 ml) was added to a solution of a compound of formula 1 (12 g) in dry ACCN (100 ml). The solution was stirred at room temperature for 1 h30. First 4-Amino-biphenyl (10.4 gr) as solid and then DiPEA (18.59 ml) by syringe was added to then solution and stirred at RT for two hours. The solution became cloudy after 10 min. The reaction mixture was filtered and washed with cold acetonitrile and powder dried over frit. Pale yellow Crystals was collected to yield the compound of formula 31 (15 g, 75%). The compound of formula 31 (3.5 gr) was then suspended in ACCN (20 ml) and 33 ml of a NaOH solution (10%) in ethanol was added. The reaction mixture was stirred at room temperature for an hour. The suspension was acidified with concentrated HCl to pH 5.3 and filtered. The solvent was evaporated under vacuum and again dissolved in minimum amount of MeOH and 10 times more THF was added. Precipitation appeared in the flask during evaporation which was filtered and dried to isolate the compound of formula 30 (2.7 g, 84%) as a beige-yellow solid.

### Example 19: Synthesis of Compound of Formula 32

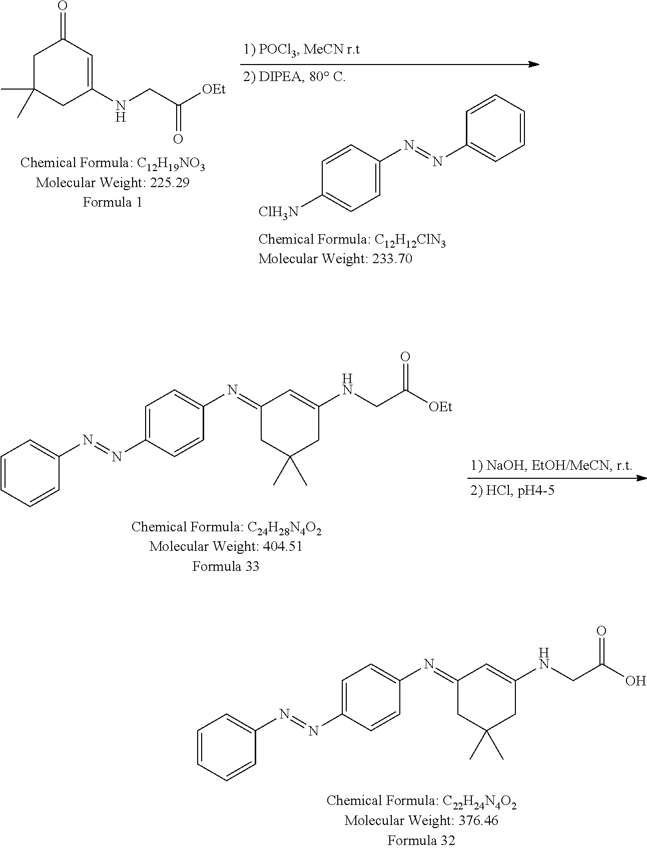

Under inert atmosphere, POCl₃ (4.55 ml) was added to a solution of a compound of formula 1 (10 g) in dry ACCN (100 ml). The solution was stirred at room temperature for 1 h30. First 4-Amino-azobenzene. HCl (10.3 gr) as solid and then DiPEA (15.5 ml) by syringe was added to the solution and stirred at 80° C. for two hours. A dark green solution was obtained. After confirming complete conversion of the starting material, ACCN was removed under vacuum. The resulting green oil was diluted in CH₂Cl₂ (100 ml), extracted with water and brine, and dried with MgSO4. The solvent was evaporated, and the resulting impure compound (formula 33) powder was purified by column chromatography on 50 gr silica. Mobile phase: DCM/MeOH 100/0, 98/2, 97/3 and finally 95/5. The different fractions were obtained, dried separately, and checked by TLC. On top of column a green fraction stays and separated from desired product. The first two fractions were brown and impure. The middle fractions were very pure. The combined solids were collected to yield the compound of formula 33 (14.7 gr, 81%) as an orange solid. The compound of formula 33 (1 gr) was suspended in ACCN (10 ml) and 8 ml of a NaOH solution (10%) in ethanol was added. The reaction mixture was stirred at room temperature for an hour. The suspension was acidified with concentrated HCl to pH 5.3 and filtered. The solvent was evaporated under vacuum and again dissolved in 20 ml Methanol and stirred 20 min and then filtered to remove NaCl. minimum amount of MeOH and 10 times more THF was added. Precipitation appeared in the flask during evaporation which was filtered and dried to isolate the compound of formula 32 (0.7 g, 70%) as an orange solid.

### Example 20: Synthesis of Compound of Formula 34

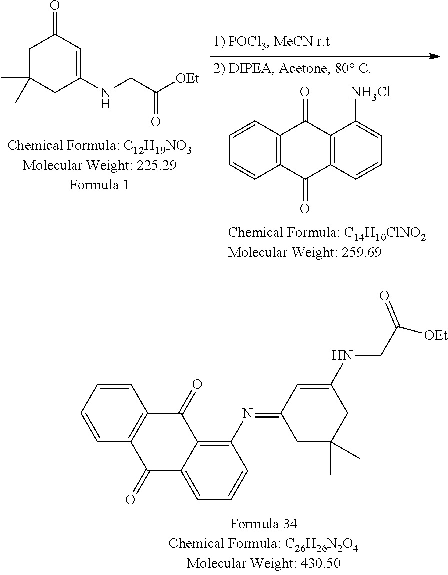

Under inert atmosphere, POCl₃ (0.91 ml) was added to a solution of a compound of formula 1 (2 g) in dry ACCN (50 ml). The solution was stirred at room temperature for 1 h30. First 1-Amino-anthraquinone (2.3 gr) as solid and then DiPEA (3.1 ml) by syringe and 50 ml of acetone was added to the solution and stirred at 80° C. for 4 hours. After confirming complete conversion of the starting material, ACCN was removed under vacuum and the resulting orange oil was diluted in CH₂Cl₂ (100 ml), extracted with water and brine, and dried with MgSO₄. The solvent was evaporated, and the resulting impure compound (formula 34) powder was purified by column chromatography on 50 gr silica. Mobile phase: DCM/MeOH 100/0 to 99/1. The different fractions obtained were dried separately and checked by TLC. The good fractions were collected to yield the compound of formula 34 (1.6 gr, 42%) as an orange-red solid.

### Example 21: Synthesis of Compound of Formula 35

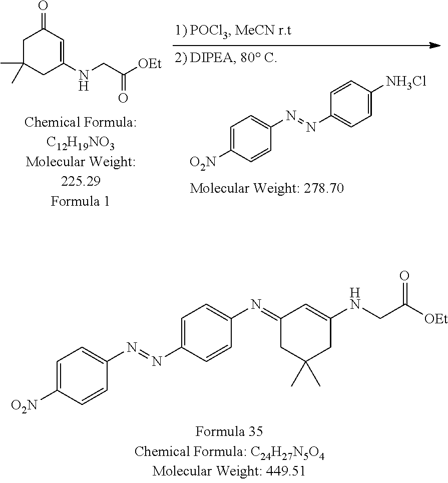

Under inert atmosphere, POCl₃ (2.28 ml) was added to a solution of a compound of formula 1 (5 g) in dry ACCN (70 ml). The solution was stirred at room temperature for 1 h30. First 4-(4-Nitrophenylazo) aniline. HCl (5.9 gr) as solid and then DiPEA (7.74 ml) by syringe was added to the solution and stirred at RT for two hours. The solution became cloudy after 10 min, and the product was completely precipitated after 2 hr of stirring. The reaction mixture was then filtered and washed with cold acetonitrile and powder dried over frit. Orange crystals were collected to yield the compound of formula 35 (6 g, 61%).

### Example 22: Synthesis of Compound of Formula 36

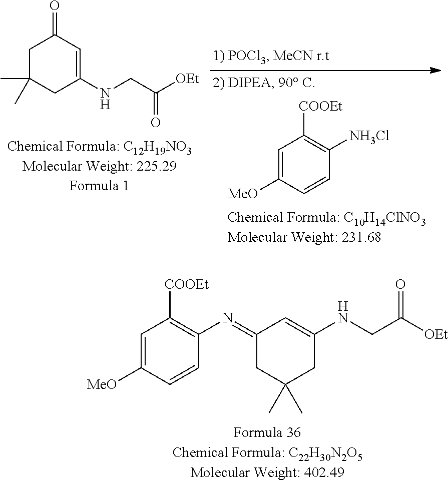

Under inert atmosphere, POCl₃ (1.37 ml) was added to a solution of a compound of formula 1 (3 g) in dry ACCN (50 ml). The solution was stirred at room temperature for 1 h30. First Ethyl 2-amino-5-methoxybenzoate. HCl (2.9 gr) as solid and then DiPEA (4.65 ml) by syringe was added to solution and stirred at 90° C. for 3 hours. After confirming complete conversion of the starting material, ACCN was removed under vacuum and the resulting orange oil was diluted in CH₂Cl₂ (100 ml), extracted with water and brine, and dried with MgSO₄. CH₂Cl₂ was evaporated totally, then 50 ml ACCN and 50 ml of MTBE was added. Half of the solvent was evaporated on Rotavapor at 50° C. and the rest was left at RT to stir. After half an hour, precipitation appeared. The reaction mixture was then filtered and washed with cold acetonitrile and powder dried over frit. White beige crystals were collected to yield the compound of formula 36 (4 g, 75%).

### Example 23: Synthesis of Compound of Formula 37

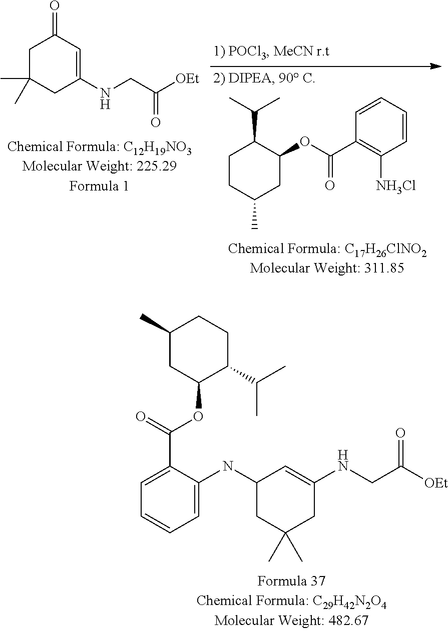

Under inert atmosphere, POCl₃ (2.28 ml) was added to a solution of a compound of formula 1 (5 g) in dry ACCN (50 ml). The solution was stirred at room temperature for 1 h30. First Menthyl anthranilate. HCl (7 gr) as solid and then DiPEA (7.74 ml) by syringe was added to the solution and stirred at 80° C. for 3 hours. After confirming complete conversion of the starting material, ACCN was removed under vacuum and the resulting oil was diluted in CH₂Cl₂ (100 ml), extracted with water and brine, and dried with MgSO₄. The solvent was evaporated, and the resulting impure compound (formula 37) powder was purified by column chromatography on 50 gr silica. Mobile phase: DCM/MeOH 100/0, 98/2 and finally 97/3. The different fractions obtained were dried separately and checked by TLC. The good fractions were collected to yield the compound of formula 37 (9.7 gr, 90%) as a yellow solid.

### Example 24: Synthesis of Compound of Formula 38

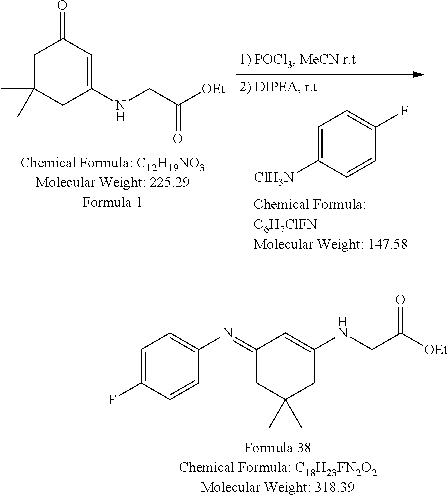

Under inert atmosphere, POCl₃ (2.28 ml) was added to a solution of a compound of formula 1 (5 g) in dry ACCN (70 ml). The solution was stirred at room temperature for 1 h30. First Fluro-aniline. HCl (3.1 gr) as solid and then DiPEA (7.74 ml) by syringe was added to then solution and stirred at RT for 2 hours. The solution became cloudy after 10 min, and the product was completely precipitated after 2 hr of stirring. The reaction mixture was filtered and washed with cold acetonitrile and powder dried over frit. White crystals were collected to yield the compound of formula 38 (4.5 g, 64%).

### Example 25: Synthesis of Compound of Formula 39

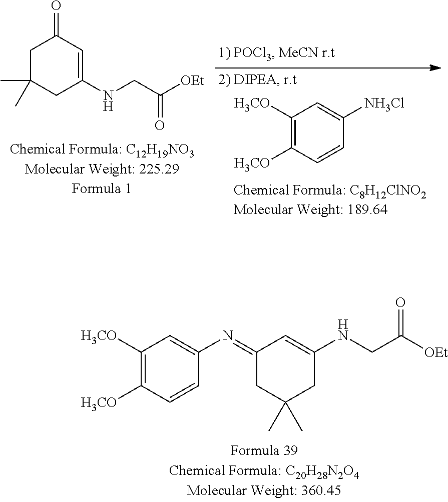

Under inert atmosphere, POCl₃ (2.73 ml) was added to a solution of a compound of formula 1 (6 g) in dry AcCN (70 ml). The solution was stirred at room temperature for 1 h30. First 3,4-dimethoxy-aniline. HCl (5 gr) as solid and then DiPEA (9.3 ml) by syringe was added to the solution and stirred at RT for 4 hours. After confirming complete conversion of the starting material, AcCN was removed under vacuum and the resulting oil was diluted in CH₂Cl₂ (100 ml), extracted with water and brine, and dried with MgSO₄. The solvent was evaporated totally and solvent mixture of AcCN/MTBE in ratio of 1/5 was added and stirred overnight. The solution became cloudy overnight, and product was completely precipitated. The reaction mixture was then filtered and washed with cold acetonitrile and powder dried over frit. Yellow crystals were collected to yield the compound of formula 39 (6.9, 71%).

### Example 26: Synthesis of Compound of Formula 40

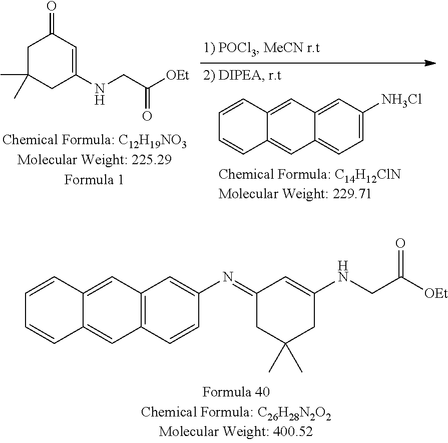

Under inert atmosphere, POCl₃ (1.82 ml) was added to a solution of a compound of formula 1 (4 g) in dry AcCN (70 ml). The solution was stirred at room temperature for 1 h30. First 2-Aminoanthracene. HCl (4 gr) as solid and then DiPEA (6.2 ml) by syringe was added to the solution and stirred at RT for 2 hours. The solution became cloudy after 10 min, and the product was completely precipitated after 2 hr of stirring. The reaction mixture was filtered and washed with cold acetonitrile and powder dried over frit. Yellow-green crystals were collected to yield the compound of formula 40 (6 g, 84%).

### Example 27: Synthesis of Compound of Formula 41

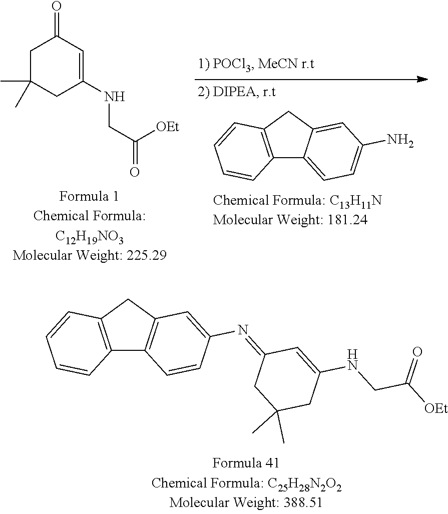 [SMILES: CCOC(=O)CNC1=CC(=O)CC(C)(C)C1 ; Nc1ccc2c(c1)Cc1ccccc1-2 ; CCOC(=O)CNC1=C/C(=N/c2ccc3c(c2)Cc2ccccc2-3)CC(C)(C)C1]

Under inert atmosphere, POCl₃ (2.28 ml) was added to a solution of a compound of formula 1 (5 g) in dry AcCN (70 ml). The solution was stirred at room temperature for 1 h30. First 4-Aminoflourene (3.8 gr) as solid and then DiPEA (7.7 ml) by syringe was added to the solution and stirred at RT for 2 hours. The solution became cloudy after 10 min, and the product was completely precipitated after 2 hr of stirring. The reaction mixture was then filtered and washed with cold acetonitrile and powder dried over frit. Beige crystals were collected to yield the compound of formula 41 (4.4 g, 51%).

### Example 28: Synthesis of Compound Formula 42

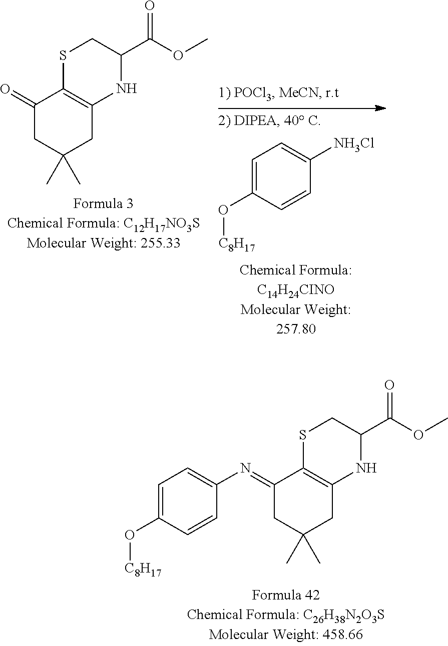

Under inert atmosphere, POCl₃ (3.81 ml) was added to a solution of a compound of formula 3 (10 g) in dry AcCN (70 ml). The solution was stirred at room temperature for 1 h30. First 1-Octyloxy-amine, HCl (10 gr) as solid and then DiPEA (18.8 ml) by syringe was added to the solution and stirred at RT for 1 hr and 2 hr at 40° C. After confirming complete conversion of the starting material, AcCN was removed under vacuum and the resulting oil was diluted in CH₂Cl₂ (200 ml), extracted with water and brine, and dried with MgSO₄. The solvent was evaporated, and the resulting impure compound (formula 42) powder was purified by column chromatography on 50 gr silica. Mobile phase: DCM/MeOH 100/0, 99/land finally 97/3. The different fractions obtained were dried separately and checked by TLC. The first two fractions were brown and impure. The combined solids were collected to yield the compound of formula 42 (14.8 g, 82%) as a yellow solid.

### Example 29: Synthesis of Compound of Formula 43

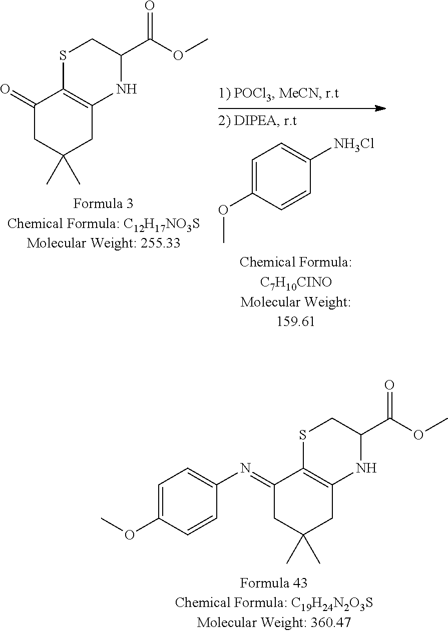

Under inert atmosphere, POCl₃ (3.21 ml) was added to a solution of a compound of formula 3 (8 g) in dry AcCN (70 ml). The solution was stirred at room temperature for 1 h30. First Anisidine. HCl (5 gr) as solid and then DiPEA (16.38 ml) by syringe was added to solution and stirred at RT overnight. After confirming complete conversion of the starting material. AcCN was removed under vacuum and the resulting oil was diluted in CH₂Cl₂ (200 ml), extracted with water and brine, and dried with MgSO₄. The solvent was evaporated, and the resulting impure compound (formula 43) powder was purified by crystallization in acetonitrile/MTBE, 50/250. The combined solids were collected to yield the compound of formula 43 (5.3 g, 47%) as a beige solid.

### Example 30: Synthesis of Compound of Formula 44

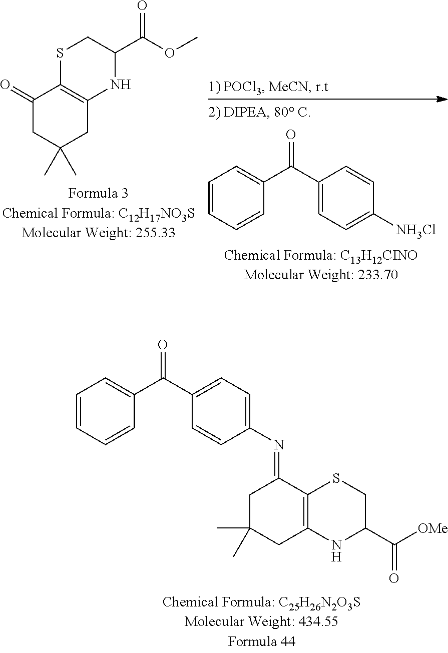

Under inert atmosphere, POCl₃ (2.41 ml) was added to a solution of a compound of formula 3 (6 g) in dry AcCN (70 ml). The solution was stirred at room temperature for 1 h30. First 4-amino-benzophenone. HCl (5.5 gr) as solid and then DiPEA (8.2 ml) by syringe was added to the solution and stirred at RT for 1 hr and 1 hr at 80° C. After confirming complete conversion of the starting material, AcCN was removed under vacuum and the resulting oil was diluted in CH₂Cl₂ (200 ml), extracted with water and brine, and dried with MgSO₄. The solvent was evaporated, and the resulting impure compound (formula 44) powder was purified by column chromatography on 50 gr silica. Mobile phase: DCM/MeOH 100/0, 99/land finally 97/3. The different fractions obtained were dried separately and checked by TLC. The first fraction was brown and impure. The combined solids were collected to yield the compound of formula 44 (9 g, 88%) as a yellow solid.

### Example 31: Synthesis of Compound of Formula 45

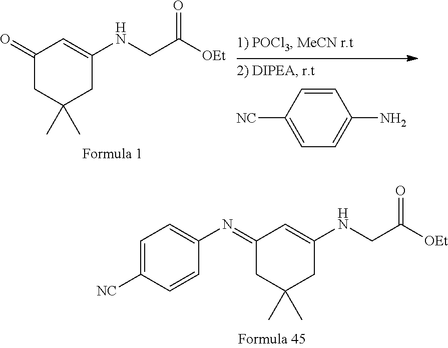 [SMILES: CCOC(=O)CNC1=CC(=O)CC(C)(C)C1 ; N#Cc1ccc(N)cc1 ; CCOC(=O)CNC1=C/C(=N/c2ccc(C#N)cc2)CC(C)(C)C1]

Under inert atmosphere, POCl₃ (2.28 ml) was added to a solution of a compound of formula 1 (5 g) in dry acetonitrile (25 ml). The solution was stirred at RT for 1 hr. 4-Aminobenzonitrile (2.5 gr) as solid and DiPEA (10 ml) by syringe were added to the solution and stirred at RT for 3 hr. LCMS showed 25% conversion. The reaction mixture was then heated to 95° C. for 4 hr. LCMS showed 85% conversion. The reaction mixture became cold and was stirred at RT overnight. Precipitation was formed, filtered, and washed with cold acetonitrile and powder dried over frit. A beige powder was collected to yield the compound of formula 45 (4 gr, 55%).

### Example 32—Synthesis of Compound of Formula 46

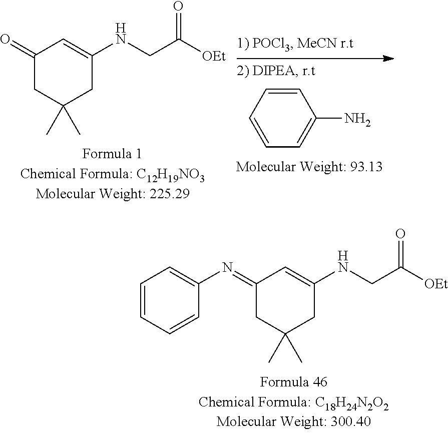 [SMILES: CCOC(=O)CNC1=CC(=O)CC(C)(C)C1 ; Nc1ccccc1 ; CCOC(=O)CNC1=C/C(=N/c2ccccc2)CC(C)(C)C1]

Under inert atmosphere, POCl₃ (2 ml) was added to a solution of a compound of formula 1 (4 g) in dry AcCN (20 ml). The solution was stirred at room temperature for 1 h30. First aniline (2 ml) and then DiPEA (7 ml) by syringe was added to the solution and stirred at RT for 3 hours. The solution became cloudy after 10 min, and the product was completely precipitated after 2 hr of stirring. The reaction mixture was filtered and washed with cold acetonitrile and powder dried over frit. White crystals were collected to yield the compound of formula 46 (3.5 g, 67%).

### Example 33: Synthesis of Compound of Formula 47

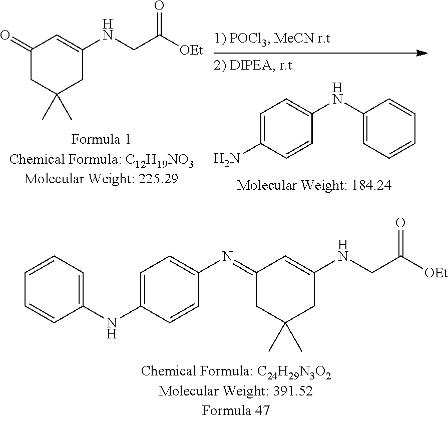 [SMILES: CCOC(=O)CNC1=CC(=O)CC(C)(C)C1 ; Nc1ccc(Nc2ccccc2)cc1 ; CCOC(=O)CNC1=C/C(=N/c2ccc(Nc3ccccc3)cc2)CC(C)(C)C1]

Under inert atmosphere, POCl₃ (5 ml) was added to a solution of a compound of formula 1 (10 g) in dry AcCN (40 ml). The solution was stirred at room temperature for 2 h. 4-Aminodiphenylamine (8 gr) as solid and DiPEA (15 ml) by syringe were added to solution and stirred at RT for 4 hr. Solution became cloudy after 2 hr. It was then cooled down in a fridge for an hour. The reaction mixture was then filtered and washed with cold acetonitrile and powder dried over frit. Pistachio green crystals were collected to yield the compound of formula 47 (15 g, 70%).

### Example 34: Synthesis of Compound of Formula 48

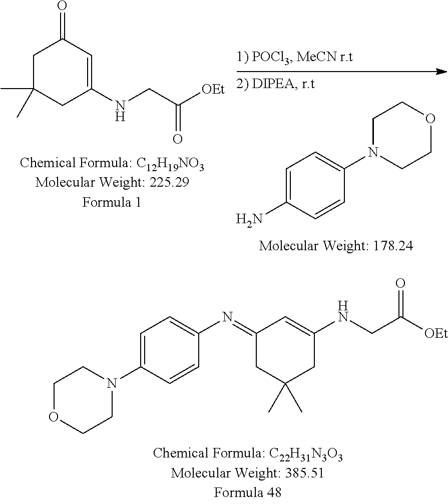 [SMILES: CCOC(=O)CNC1=CC(=O)CC(C)(C)C1 ; Nc1ccc(N2CCOCC2)cc1 ; CCOC(=O)CNC1=C/C(=N/c2ccc(N3CCOCC3)cc2)CC(C)(C)C1]

Under inert atmosphere, POCl₃ (1.5 ml) was added to a solution of a compound of formula 1 (3 g) in dry AcCN (10 ml). The solution was stirred at room temperature for 1 h. 4-Morpholinoaniline (3 gr) as solid and DiPEA (5 ml) by syringe were added to the solution and stirred at RT for 4 hr. The solution became cloudy after 1 hr. It was then cooled down in a fridge for an hour. The reaction mixture was then filtered and washed with cold acetonitrile and powder dried over frit. Yellow crystals were collected to yield the compound of formula 48(4 g, 70%).

### Example 35: Synthesis of Compound of Formula 49

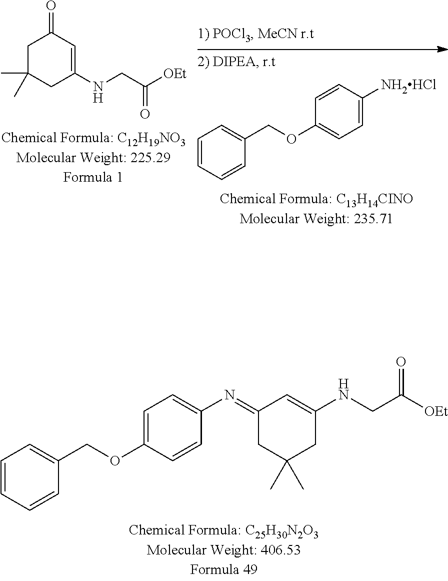

Under inert atmosphere, POCl₃ (4 ml) was added to a solution of a compound of formula 1 (10 g) in dry AcCN (40 ml). The solution was stirred at room temperature for 3 h. 4-benzyloxy-aniline. hydrochloride (10 gr) as solid and DiPEA (20 ml) by syringe were added to the solution and stirred at RT for 4 hr. The solution became cloudy after 1 min. The solution was then cooled down in a fridge for an hour. The reaction mixture was then filtered and washed with cold acetonitrile and powder dried over frit. Beige crystals were collected to yield the compound of formula 49 (15 g, 90%).

### Example 36: Synthesis of Compound of Formula 50

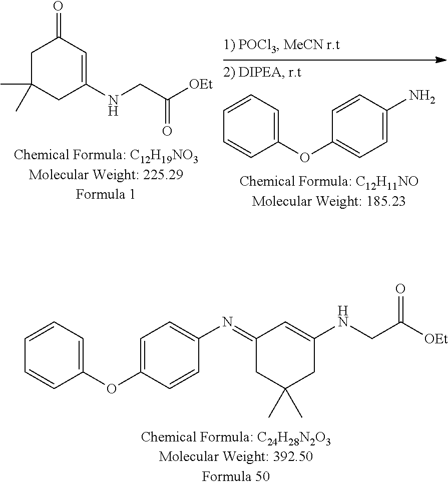

Under inert atmosphere, POCl₃ (3 ml) was added to a solution of a compound of formula 1 (5 g) in dry AcCN (40 ml). The solution was stirred at room temperature for 1 h. 4-phenoxyaniline (3.9 gr) as solid and DiPEA (7 ml) by syringe were added to then solution and stirred at RT for 4 hr. The solution became cloudy immediately. The reaction mixture was then filtered and washed with cold acetonitrile and powder dried over frit. White crystals were collected to yield the compound of formula 50 (7.2 g, 95%).

### Example 37: Synthesis of Compound of Formula 51

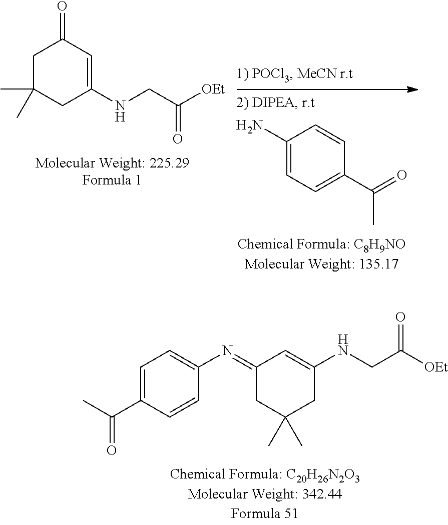

Under inert atmosphere, POCl₃ (5 ml) was added to a solution of a compound of formula 1 (10 g) in dry AcCN (40 ml). The solution was stirred at room temperature for 1 h. 4-Aminoacetophenone (6 gr) as solid and DiPEA (14 ml) by syringe were added to the solution and stirred at RT for 4 hr and 1 hr stirring at 75° C. The solution was then placed in a fridge overnight, and precipitation was formed. The reaction mixture was then filtered and washed with cold acetonitrile and powder dried over frit. Beige crystals were collected to yield the compound of formula 51 (7 g, 50%).

### Example 38: Synthesis of Compound of Formula 52

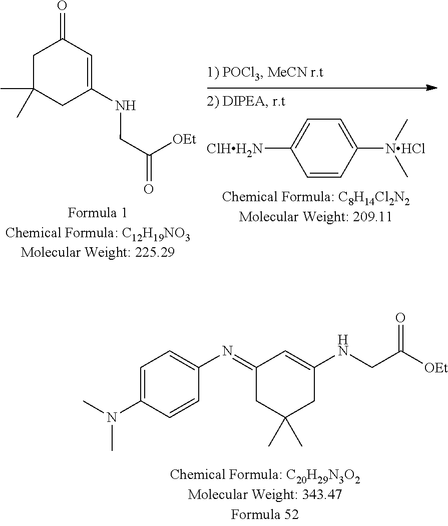

Under inert atmosphere, POCl₃ (3 ml) was added to a solution of a compound of formula 1 (5 g) in dry AcCN (20 ml). The solution was stirred at room temperature for 1 h. 4-phenoxyaniline (3.9 gr) as solid and DiPEA (7 ml) by syringe were added to the solution and stirred at RT for 5 hr. The solution became cloudy immediately. The reaction mixture was then filtered and washed with cold acetonitrile and powder dried over frit. White crystals were collected to yield the compound of formula 52 (8 g, 85%).

### Example 39: Synthesis of Compound of Formula 53

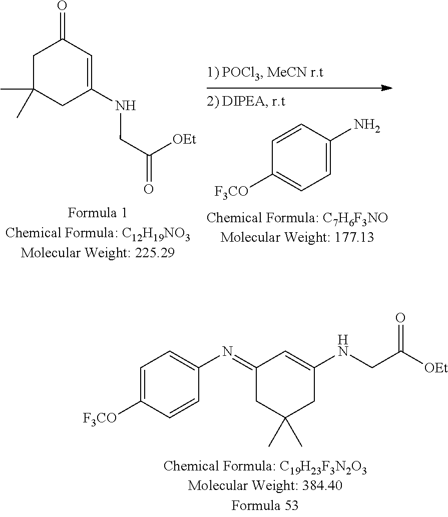 [SMILES: CCOC(=O)CNC1=CC(=O)CC(C)(C)C1 ; Nc1ccc(OC(F)(F)F)cc1 ; CCOC(=O)CNC1=C/C(=N/c2ccc(OC(F)(F)F)cc2)CC(C)(C)C1]

Under inert atmosphere, POCl₃ (4 ml) was added to a solution of a compound of formula 1 (10 g) in dry AcCN (40 ml). The solution was stirred at room temperature for 1 h. 4-(Trifluoromethoxy) (8 gr) as solid and DiPEA (15 ml) by syringe were added to solution and stirred at RT for 4 hr. The solution became cloudy after 10 min. The solution was then cooled down in a fridge for an hour. The reaction mixture was filtered and washed with cold acetonitrile and powder dried over frit. Beige crystals were collected to yield the compound of formula 53 (8 g, 50%).

### Example 40: Synthesis of Compound of Formula 54

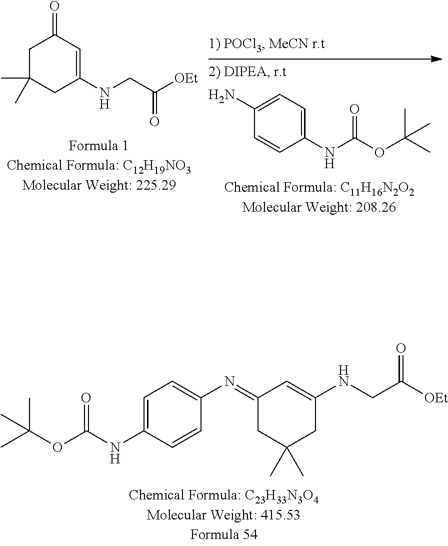 [SMILES: CCOC(=O)CNC1=CC(=O)CC(C)(C)C1 ; CC(C)(C)OC(=O)Nc1ccc(N)cc1 ; CCOC(=O)CNC1=C/C(=N/c2ccc(NC(=O)OC(C)(C)C)cc2)CC(C)(C)C1]

Under inert atmosphere, POCl₃ (2 ml) was added to a solution of a compound of formula 1 (5 g) in dry AcCN (20 ml). The solution was stirred at room temperature for 1 h. Tert-Butyl (4-Aminophenyl) carbamate (4 gr) as solid and DiPEA (8 ml) by syringe were added to solution and stirred at RT for 4 hr and at 50° C. for 1 hr and then solvent was evaporated to half and MTBE was added (50/50) to stir overnight. The product completely was precipitated during overnight stirring. The reaction mixture was then filtered and washed with cold acetonitrile and powder dried over frit. Beige crystals were collected to yield the compound of formula 54 (5 g, 65%).

### Example 41: Synthesis of Compound of Formula 55

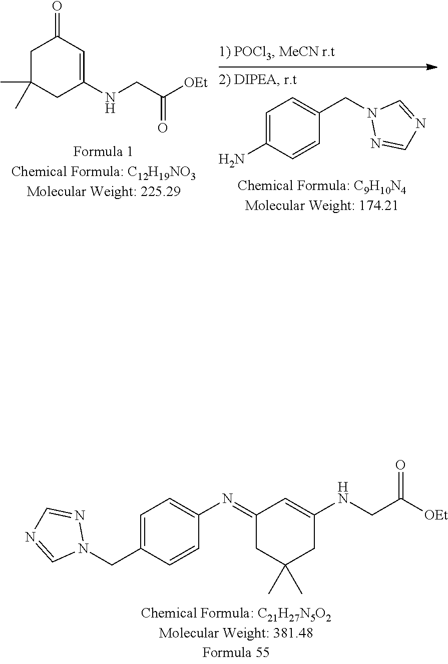 [SMILES: CCOC(=O)CNC1=CC(=O)CC(C)(C)C1 ; Nc1ccc(Cn2cncn2)cc1 ; CCOC(=O)CNC1=C/C(=N/c2ccc(Cn3cncn3)cc2)CC(C)(C)C1]

Under inert atmosphere, POCl₃ (4.55 ml) was added to a solution of a compound of formula 1 (10 g) in dry AcCN (40 ml). The solution was stirred at room temperature for 1 h; then 1(4-aminobenzyl)-1,2,4-triazole (7.4 gr) as solid and DiPEA (15.5 ml) by syringe were added to solution and stirred at RT for 4 hr. The solution became cloudy after stirring for 24 hr. The reaction mixture was filtered and washed with cold acetonitrile and powder dried over frit. White crystals were collected to yield the compound of formula 55 (7.5 g, 64%).

### Example 42: Synthesis of Compound of Formula 56

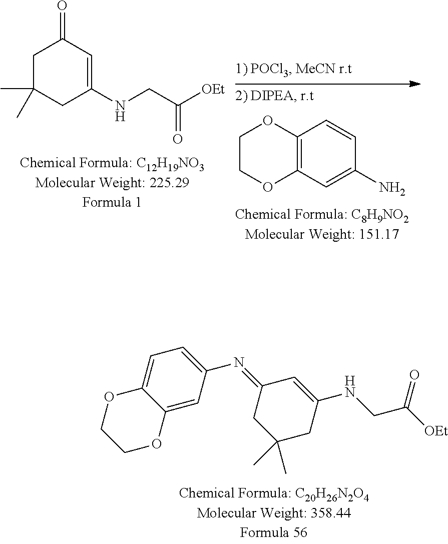 [SMILES: CCOC(=O)CNC1=CC(=O)CC(C)(C)C1 ; Nc1ccc2c(c1)OCCO2 ; CCOC(=O)CNC1=C/C(=N/c2ccc3c(c2)OCCO3)CC(C)(C)C1]

Under inert atmosphere, POCl₃ (2.28 ml) was added to a solution of a compound of formula 1 (5 g) in dry AcCN (20 ml). The solution was stirred at room temperature for 1 h. (3,4-Ethylendioxy) aniline (2.6 ml) as solid and DiPEA (10 ml) by syringe were added to the solution and stirred at RT for 10 hr. The solution became cloudy after 30 min. The reaction mixture was then filtered and washed with cold acetonitrile and powder dried over frit. White crystals were collected to yield the compound of formula 56 (6 g, 70%).

### Example 43: Synthesis of Compound of Formula 57

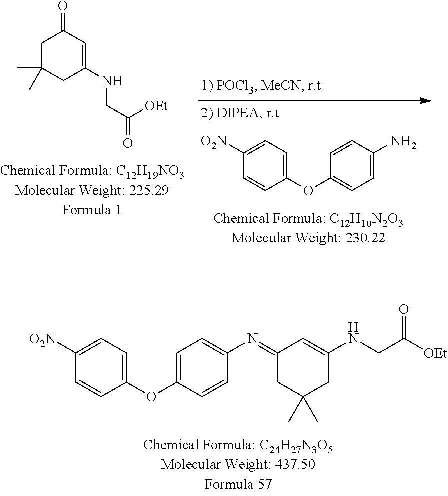 [SMILES: CCOC(=O)CNC1=CC(=O)CC(C)(C)C1 ; Nc1ccc(Oc2ccc([N+](=O)[O-])cc2)cc1 ; CCOC(=O)CNC1=C/C(=N/c2ccc(Oc3ccc([N+](=O)[O-])cc3)cc2)CC(C)(C)C1]

Under inert atmosphere, POCl₃ (1 ml) was added to a solution of a compound of formula 1 (2 g) in dry AcCN (10 ml). The solution was stirred at room temperature for 1 h. 4-Nitro-Phenoxyaniline (2.4 gr) as solid and DiPEA (3.87 ml) by syringe were added to the solution and stirred at RT for 4 hr. The solution became cloudy after 1 hr. The reaction mixture was then filtered and washed with cold acetonitrile and powder dried over frit. White-beige crystals were collected to yield the compound of formula 57 (4 g, 70%).

### Example 44: Synthesis of Compound of Formula 58

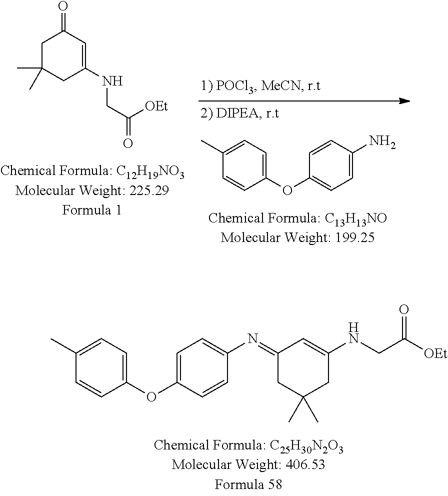 [SMILES: CCOC(=O)CNC1=CC(=O)CC(C)(C)C1 ; Cc1ccc(Oc2ccc(N)cc2)cc1 ; CCOC(=O)CNC1=C/C(=N/c2ccc(Oc3ccc(C)cc3)cc2)CC(C)(C)C1]

Under inert atmosphere, POCl₃ (2 ml) was added to a solution of a compound of formula 1 (3 g) in dry AcCN (10 ml). The solution was stirred at room temperature for 1 h. 4-Methyl-Phenoxyaniline (2.5 gr) as solid and DiPEA (4.65 ml) by syringe were added to the solution and stirred at RT for 4 hr. The solution became cloudy after 1 hr. The reaction mixture was filtered and washed with cold acetonitrile and powder dried over frit. White-light blue crystals were collected to yield the compound of formula 58 (3 g, 65%).

### Example 45: Synthesis of Compound of Formula 59

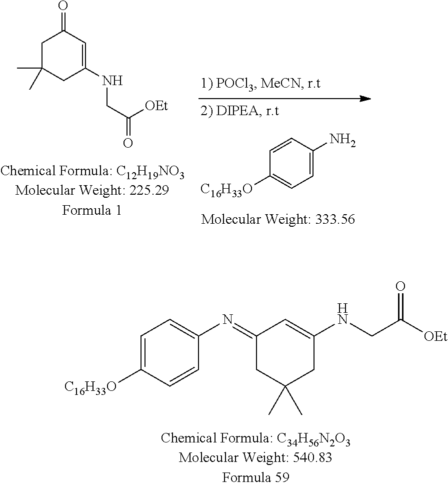

Under inert atmosphere, POCl₃ (1.5 ml) was added to a solution of a compound of formula 1 (2 g) in dry AcCN (10 ml). The solution was stirred at room temperature for 1 h. P-Hexadecyloxyaniline (3 gr) as solid and DiPEA (3.1 ml) by syringe were added to the solution and stirred at RT for 4 hr. The solution became cloudy after 1 hr. The reaction mixture was then filtered and washed with cold acetonitrile and powder dried over frit. Gray crystals were collected to yield the compound of formula 59 (4 g, 66%).

### Example 46: Synthesis of Compound of Formula 60

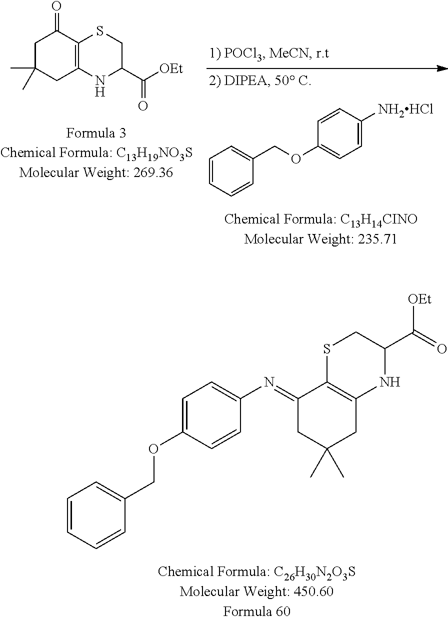

Under inert atmosphere, POCl₃ (5 ml) was added to a solution of a compound of formula 3 (10 g) in dry AcCN (40 ml). The solution was stirred at room temperature for 1 h30. First, 4-benzyloxy-aniline. Hydrochloride (10 gr) as solid and then DiPEA (12.6 ml) by syringe were added to the solution and stirred at 65° C. for 2 hours. After confirming complete conversion of the starting material, AcCN was removed under vacuum and the resulting oil was diluted in CH₂Cl₂ (300 ml), extracted with water and brine, and dried with MgSO₄. The solvent was then evaporated, and the resulting impure compound (formula 60) was purified by column chromatography on 50 gr silica. Mobile phase: DCM/MeOH 100/0, 99/land finally 97/3. The different fractions obtained were dried separately and checked by TLC. The first fractions were impure by non-reacted amine. The combined solids were collected to yield the compound of formula 60 (15 g, 75%) as an orange-brown solid.

### Example 47: Synthesis of Compound of Formula 61

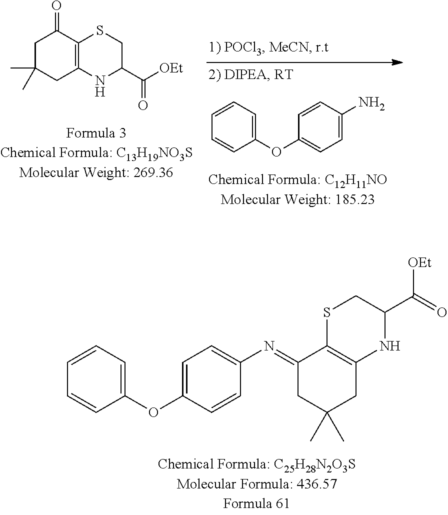 [SMILES: CCOC(=O)C1CSC2=C(CC(C)(C)CC2=O)N1 ; Nc1ccc(Oc2ccccc2)cc1 ; CCOC(=O)C1CSC2=C(CC(C)(C)C/C2=N\c2ccc(Oc3ccccc3)cc2)N1]

Under inert atmosphere, POCl₃ (5 ml) was added to a solution of a compound of formula 3 (10 g) in dry AcCN (40 ml). The solution was stirred at room temperature for 1 h30. First, 4-Phenoxyaniline (5 gr) as solid and then DiPEA (15 ml) by syringe were added to the solution and stirred at RT for 3 hours. After confirming complete conversion of the starting material, AcCN was removed under vacuum to ⅓ and MTBA (4 times) was added to stir overnight. Precipitation was formed and the reaction mixture was filtered and washed with cold acetonitrile and powder dried over frit. A yellow solid was collected to yield the compound of formula 61 (11 g, 60%).

### Example 48: Synthesis of Compound of Formula 62

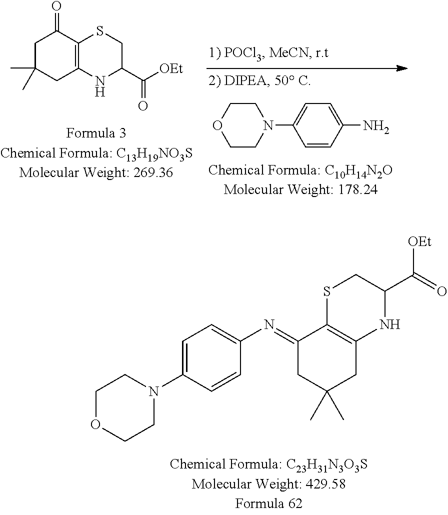 [SMILES: CCOC(=O)C1CSC2=C(CC(C)(C)CC2=O)N1 ; Nc1ccc(N2CCOCC2)cc1 ; CCOC(=O)C1CSC2=C(CC(C)(C)C/C2=N\c2ccc(N3CCOCC3)cc2)N1]

Under inert atmosphere, POCl₃ (2.5 ml) was added to a solution of a compound of formula 3 (5 g) in dry AcCN (20 ml). The solution was stirred at room temperature for 1 h30. First 4-Aminomorphoaniline (5 gr) as solid and then DiPEA (6.5 ml) by syringe was added to solution and stirred at 50° C. for 2 hours. After confirming complete conversion of the starting material, AcCN was removed under vacuum and the resulting oil was diluted in CH₂Cl₂ (300 ml), extracted with water and brine, and dried with MgSO₄. The solvent was evaporated, and the resulting impure compound of formula 62 was purified by column chromatography on 50 gr silica. Mobile phase: DCM/MeOH 100/0, 99/land finally 97/3. The different fractions obtained were dried separately and checked by TLC. The first fractions were impure by non-reacted amine. The yellow-brown solid was collected to yield the compound of formula 62 (2 g, 35%).

### Example 49: Synthesis of Compound of Formula 63

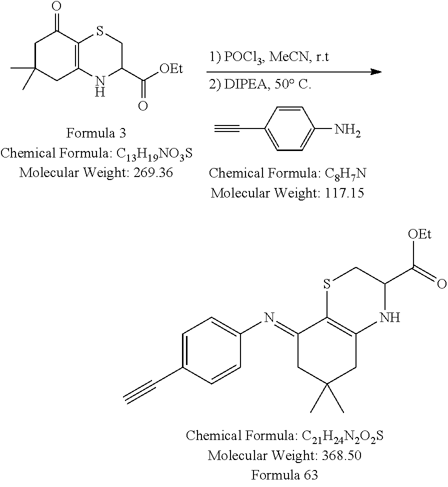

Under inert atmosphere, POCl₃ (4 ml) was added to a solution of a compound of formula 3 (5 g) in dry AcCN (20 ml). The solution was stirred at room temperature for 1 h30. First 4-Ethynylaniline (2 gr) as solid and then DiPEA (6.5 ml) by syringe was added to the solution and stirred at 50° C. for 2 hours. After confirming complete conversion of the starting material, AcCN was removed under vacuum and the resulting oil was diluted in CH₂Cl₂ (300 ml), extracted with water and brine, and dried with MgSO₄. The solvent was evaporated, and the resulting impure compound of formula 63 was purified by column chromatography on 50 gr silica. Mobile phase: DCM/MeOH 100/0, 99/land finally 97/3. The different fractions obtained were dried separately and checked by TLC. The first fractions were impure by non-reacted amine. The yellow-brown solid was collected to yield the compound of formula 63 (2 g, 45%).

### Example 50: Synthesis of Compound of Formula 64

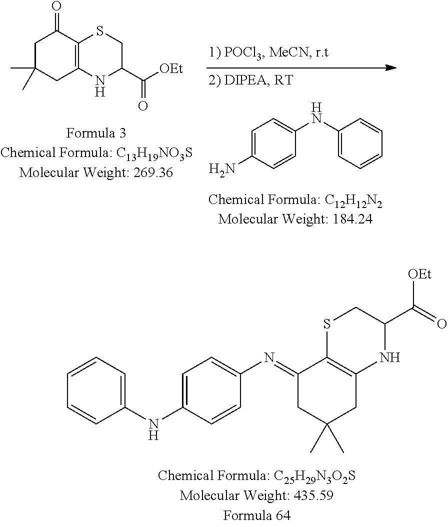 [SMILES: CCOC(=O)C1CSC2=C(CC(C)(C)CC2=O)N1 ; Nc1ccc(Nc2ccccc2)cc1 ; CCOC(=O)C1CSC2=C(CC(C)(C)C/C2=N\c2ccc(Nc3ccccc3)cc2)N1]

Under inert atmosphere, POCl₃ (5 ml) was added to a solution of a compound of formula 3 (10 g) in dry AcCN (40 ml). The solution was stirred at room temperature for 1 h30. First, 4-Amino diphenylamine (6.6 gr) as solid, and then DiPEA (12.6 ml) by syringe were added to solution and stirred at RT for 13 hours. After confirming complete conversion of the starting material, AcCN was removed under vacuum and the resulting oil was diluted in CH₂Cl₂ (300 ml), extracted with water and brine, and dried with MgSO₄. The solvent was evaporated and the solid was redissolved in MeOH, and MTBE/CH₂Cl₂ was added to stir overnight. Crystals appeared, which were filtered and washed with cold DCM and powder dried over frit. A light green solid was collected to yield the compound of formula 64 (10 g, 64%).

### Example 51: Synthesis of Compound of Formula 65

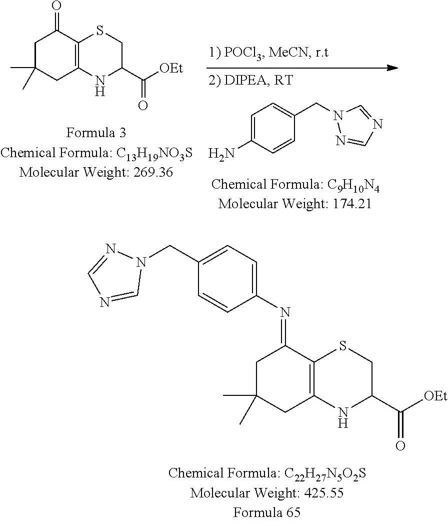 [SMILES: CCOC(=O)C1CSC2=C(CC(C)(C)CC2=O)N1 ; Nc1ccc(Cn2cncn2)cc1 ; CCOC(=O)C1CSC2=C(CC(C)(C)C/C2=N\c2ccc(Cn3cncn3)cc2)N1]

Under inert atmosphere, POCl₁₃ (3 ml) was added to a solution of a compound of formula 3 (10 g) in dry AcCN (40 ml). The solution was stirred at room temperature for 1 h30. First, 1(4-aminobenzyl)-1,2,4-triazole (9 gr) was added as solid, and then DiPEA (12.6 ml) by syringe was added to the solution and stirred at RT for 3 hours. After confirming complete conversion of the starting material, solvent was evaporated to half and MTBE was added to stir overnight. Precipitation appeared, which was filtered and washed with cold DCM and powder dried over frit. A brown solid was collected to yield the compound of formula 65 (8.3 g, 63%).

### Example 52: Synthesis of Compound of Formula 66

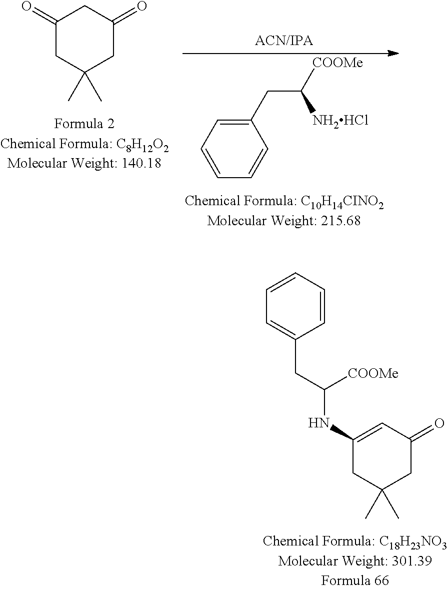

Pyridine (3.5 ml), a compound of formula 2 (6 gr) and phenylalanine methyl ester. HCl (9.6) were combined in 80 ml of CH₃CN and 30 ml of IPA. The suspension was heated (oil bath, 80° C.) and stirred until the solution became hemogenic. After 2 hr, LCMS showed 82% of product of formula 66 and the rest was the compound of formula 2. Therefore, another 18% of phenylalanine methyl ester. HCl (2 gr) and pyridine (0.7 ml) were added. LCMS after 1 hr showed 92% of product and then rest is the compound of formula 2. Reaction was stopped. The solvent was removed under vacuum. 500 ml water was added to flask and stirred for 1 hr. A beige precipitate appeared in flask. The precipitation was filtered and washed with cold water and powder dried over frit, followed by rotavap. The beige solid was collected to yield the compound of formula 66 (11 g, 85%).

### Example 53: Synthesis of Compound of Formula 67

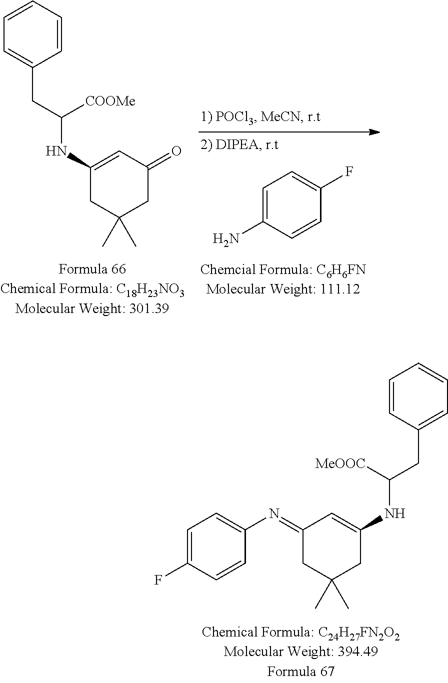

Under inert atmosphere, POCl₃ (1.7 ml) was added to a solution of a compound of formula 66 (5 g) in dry AcCN (20 ml). The solution was stirred at room temperature for 1 h. 4-Fluoroaniline (4 ml) as solid and DiPEA (5.8 ml) by syringe were added to the solution and stirred at RT for 4 hr. After obtaining full conversion, the reaction mixture was placed in a fridge overnight. Precipitation was formed, and the reaction mixture was filtered and washed with cold acetonitrile and powder dried over frit. White crystals were collected to yield the compound of formula 67 (4.5 g, 70%).

### Example 54: Synthesis of Compound of Formula 68

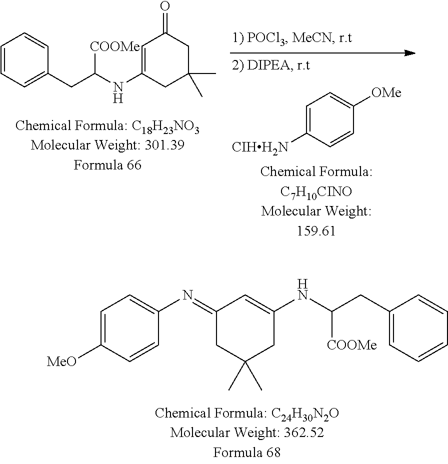

Under inert atmosphere, POCl₃ (3 ml) was added to a solution of a compound of formula 66 (5 g) in dry AcCN (20 ml). The solution was stirred at room temperature for 1 h. Anisidine. HCl (2 gr) as solid and DiPEA (5 ml) by syringe were added to the solution and stirred at RT for 4 hr and at 80° C. for 1 hr. After confirming complete conversion of the starting material, AcCN was removed under vacuum and the resulting oil was diluted in CH₂Cl₂ (300 ml), extracted with water and brine, and dried with MgSO₄. The solvent was evaporated, and the resulting impure compound of formula 68 was purified by column chromatography on 50 gr silica. Mobile phase: DCM/MeOH 100/0, 99/land finally 97/3. The different fractions obtained were dried separately and checked by TLC. The first fractions were impure by non-reacted amine. A yellow-brown solid was collected to yield the compound of formula 68 (5 g, 79%).

### Example 55: Synthesis of Compound of Formula 69

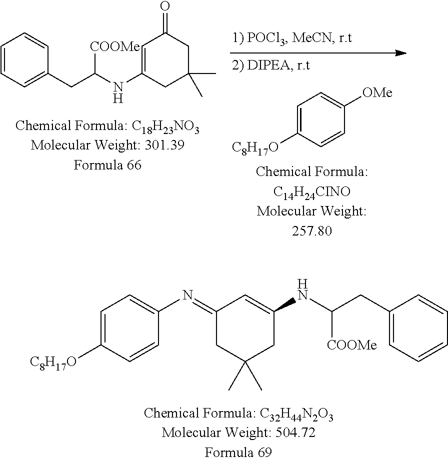

Under inert atmosphere, POCl₁₃ (0.7 ml) was added to a solution of a compound of formula 66 (2 g) in dry AcCN (10 ml). The solution was stirred at room temperature for 1 h. Octyloxyaniline. HCl (1.6 gr) as solid and DiPEA (2.5 ml) by syringe were added to the solution and stirred at RT for 4 hr and at 80° C. for 1 hr. After confirming complete conversion of the starting material, AcCN was removed under vacuum and the resulting oil was diluted in CH₂C12(300 ml), extracted with water and brine, and dried with MgSO₄. The solvent was evaporated, and the resulting impure crude compound (formula 69) was purified by column chromatography on 25 gr silica. Mobile phase: DCM/MeOH 100/0, 99/land finally 97/3. The different fractions obtained, were dried separately and checked by TLC. The first fractions were impure by non-reacted amine. A yellow-brown solid was collected to yield the compound of formula 69 (2.3 g, 60%).

### Example 56: Synthesis of Compound of Formula 70

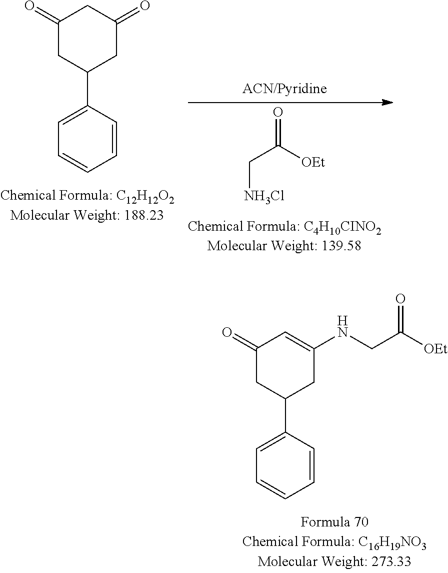

Pyridine (4.7 ml), 5-Phenyl-1,3-cyclohexanedione (10 gr) and glycine ethyl ester. HCl (7 gr) were combined in 100 ml of CH₃CN. The suspension was heated (oil bath, 95° C.) and stirred until the solution became hemogenic. After 3 hrs, the reaction was completed and stopped. The solvent was removed under vacuum. The resulting yellow oil was diluted in CH₂Cl₂ and extracted twice with water, brine and dried with MgSO₄. The crude was dissolved in hot CH₃CN and crystallized. A white solid was collected to yield the compound of formula 70 (10 g, 68%).

### Example 57: Synthesis of Compound of Formula 71

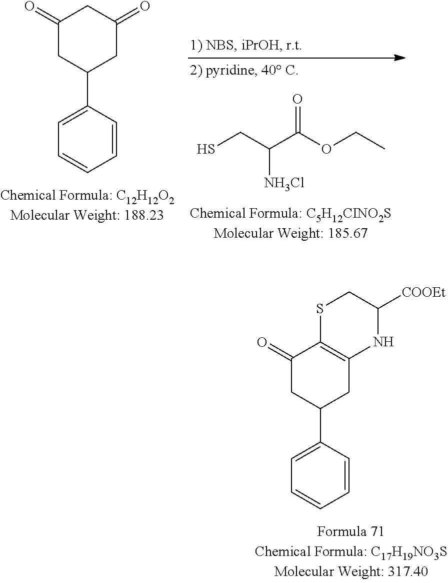

A 250 mL round bottom flask was charged with 4.6 g of Phenylcyclohexane, 1,3-Dione, 15 mL of 2-propanol (IPA) and a magnetic stir bar. NBS (5.2 g) was added portion wise over a 10-minute period by keeping an internal temperature below 20° C. The mixture was heterogenous and yielded a white slurry. The mixture was stirred for 20 minutes. Pyridine (4.2 mL) were added to the mixture and L-cysteine ethyl ester hydrochloride (5.4 g) was added portion wise keeping internal temperature below 23° C. The mixture turned yellow and homogeneous and was heated at 40° C. for 2 hours.

### Example 58: Assessing SPF of Compounds

For determining the SPF in vitro value, the protection performance of compounds according to certain embodiments of the present technology, against erythemally-effective UV radiation, largely confined to the UVB (290-320 nm) and short-wavelength UVA (320-340) region, was calculated from the measured in vitro transmittance. The in vitro UVA PF, the UVA protection (320-400 nm), was calculated from the measured in vitro transmittance after irradiation. The Critical Wavelength Value was defined as the wavelength at which the integral of the spectral absorbance curve reached 90% of the integral over the UV spectrum from 290 to 400 nm. It has been settled that this value must be equal or over 370 nm so as to classify the product as broad-spectrum. The study consisted in a comparative assay of non-treated plates against plates treated with each of the compounds and was based on the evaluation of UV-transmittance through a thin film of sunscreen sample spread on a roughened substrate, before an after exposure to a controlled dose of UV radiation from a UV source. A Kontron 933 spectrophotometer equipped with a UV source, an integrating sphere and a monochromatic light able to deliver a flow of energy between 290 and 400 nm was used. The transmittance values were measured at 1 nm intervals. A 10-4 precision laboratory balance was used to control deposited product weight. The irradiation was provided by Sunset Atlas CPS+ with standard filter. Temperature regulation of the equipment was done in the range of 25-35° C. A pre-irradiation dose of 4 times 200 J/m²-eff (800 J/m²-eff) was delivered. The substrate was the material to which the sunscreen product was applied. Polymethylmethacrylate (PMMA) plates were used and were roughened on one side to a three-dimensional surface topography of 5 micrometers. Each compound was weighted and applied evenly to the PMMA plate with a 2-phase spreading to achieve a 0.75 mg/cm² weight/surface ratio. Spreading was performed with a light spreading move for approximately 30 seconds followed by spreading with greater pressure for approximately 30 seconds. The resulting sample was left to equilibrate for 15 minutes in the dark at room temperature to ensure a self-leveling of the formula. To account for lack of photostability, a pre-irradiation was necessary. The pre-irradiation dose was 4 minimal erythema dose (MEDs), equivalent to 800 J/m²-eff. Five measurements of spectral irradiance transmitted for each wavelength through the PMMA plate covered with the sunscreen product were obtained after pre-irradiation of the sunscreen product [P1( ), P2( ), P3( ), P4( ) and P5( )]. For each compound, mean absorbance values were determined from at least three individual PMMA plates. To validate the accuracy of the results, a control product with an established SPF of 18-20, Lot 11T0313 was tested simultaneously with the compounds. SPF in vitro was calculated for each plate using the Colipa 2011 equation. A summary of the results for each of the compound is presented in Table 1 below:

TABLE 1
Assessment of SPF

| | Compound | | | | | SPF | |
|---|---|---|---|---|---|---|---|
| | (formula #) | SDF mean | UV APF | SPF P | UV APF P | Change % | ΔSPF |
| Thiazine ring | 43 | 20.84 | 4.02 | 18.59 | 3.94 | 4 | 1 |
| | 15 | 28.31 | 4.52 | 27.34 | 4.49 | 41 | 8 |
| | 17 | 31.90 | 3.66 | 25.78 | 3.17 | 54 | 12 |
| | 19 | 27.12 | 4.07 | 24.31 | 3.92 | 35 | 7 |
| Absence of | 72 | 23.61 | 2.85 | 21.12 | 2.74 | 18 | 4 |
| Thiazine ring | 29 | 32.66 | 3.36 | 27.64 | 3.13 | 63 | 13 |
| | 31 | 32.75 | 2.95 | 30.52 | 2.88 | 63 | 13 |
| | 33 | 36.23 | 4.26 | 33.16 | 4.36 | 81 | 16 |
| | 35 | 21.53 | 17.35 | 17.94 | 14.64 | 7 | 2 |
| | 37 | 23.26 | 16.91 | 20.98 | 15.51 | 16 | 3 |
| | 73 | 32.31 | 16.05 | 27.60 | 13.08 | 61 | 12 |
| | Base | 21.6 | 16.27 | 16.37 | 13.19 | 8 | 2 |

The SPF boosting effect of compounds according to certain embodiments of the present technology were also assessed in mineral base sunscreen formulations. Briefly, SPF measurements were conducted as above in plates treated with mineral base sunscreen formulations comprising 1 of a protective compound according to the present technology. The SPF measurement were compared to similar measurements obtained with identical mineral base sunscreen formulations comprising no protective compound. A summary of the results is presented in the Tables 2, 3 and 4 below:

TABLE 2
Measurements of mineral base sunscreen formulations
comprising 1% protective compound:

| | SPF | UVAPF | SPF | UVAPF | Δ | UVA/UVB | UVA/UVB |
|---|---|---|---|---|---|---|---|
| | before | before | post | post | SPF | before | post |
| Plate #1 | 125.03 | 16.7 | 116.45 | 14.90 | −8.58 | 0.76 | 0.728 |
| Plate #2 | fail | fail | fail | fail | fail | fail | fail |
| Plate #3 | 115.34 | 16.06 | 105.56 | 14.10 | −9.78 | 0.75 | 0.72 |
| Plate #4 | 124.72 | 17 | 120.62 | 15.45 | −4.10 | 0.76 | 0.73 |
| Plate #5 | 123.08 | 18.10 | 121.5 | 16.11 | −1.58 | 0.76 | 0.74 |
| Plate #6 | 109.76 | 16.87 | 101.08 | 14.81 | −8.68 | 0.76 | 0.73 |
| Plate #7 | fail | fail | fail | fail | fail | fail | fail |
| Plate #8 | 111.57 | 17.14 | 100.56 | 14.93 | −11.01 | 0.76 | 0.73 |
| Plate #9 | 110.76 | 18.16 | 106.74 | 16.46 | −4.02 | 0.76 | 0.74 |
| Plate #10 | 92.65 | 17.2 | 82.61 | 14.67 | −10.04 | 0.76 | 0.73 |
| Mean Value | 114.11 | 17.15 | 106.89 | 15.18 | −7.22 | 0.76 | 0.73 |

TABLE 3
Measurements of mineral base sunscreen formulations
without protective compound:

| | SPF | UVAPF | SPF | UVAPF | Δ | UVA/UVB | UVA/UVB |
|---|---|---|---|---|---|---|---|
| | before | before | post | post | SPF | before | post |
| Plate #1 | 43.21 | 16.76 | 41.22 | 17.23 | −1.99 | 0.724 | 0.729 |
| Plate #2 | 39.71 | 17.32 | 40.25 | 18.16 | 0.54 | 0.729 | 0.734 |
| Plate #3 | 71.43 | 16.85 | 74.04 | 18.06 | 2.61 | 0.726 | 0.732 |
| Plate #4 | 33.59 | 16.58 | 35.46 | 17.69 | 1.87 | 0.723 | 0.727 |
| Plate #5 | 46.16 | 16.75 | 47.07 | 17.08 | 0.91 | 0.724 | 0.725 |
| Plate #6 | 31.38 | 16.93 | 33.52 | 17.77 | 2.14 | 0.725 | 0.724 |
| Plate #7 | 27.95 | 17.52 | 27.73 | 17.28 | −0.22 | 0.731 | 0.728 |
| Plate #8 | 28.41 | 16.9 | 27.72 | 16.53 | −0.69 | 0.726 | 0.724 |
| Plate #9 | 46.41 | 16.58 | 44.81 | 16.14 | −1.60 | 0.724 | 0.721 |
| Plate #10 | 36.5 | 16.72 | 36.34 | 16.41 | −0.16 | 0.726 | 0.722 |
| Mean Value | 40.475 | 16.891 | 40.816 | 17.235 | 0.34 | 0.7258 | 0.7266 |

TABLE 4
Δ SPF mineral base sunscreen formulations
comprising 1% protective compound compared to
identical formulation without protective compound.

| | | | |
|---|---|---|---|
| Mean with protective compound | 106.89 | 15.18 | 0.73 |
| Mean Base | 40.82 | 17.24 | 0.73 |
| A SPF | 66.07 | | |

### Example 59: Preparation of Sunscreen Formulations Comprising Compounds of the Present Technology

Sunscreen formulations A-K that include compounds which absorb UV radiation and/or visible radiation and protect biological materials as well as non-biological materials from damaging UV radiation and/or visible radiation were prepared. A basic neutral cream was developed in order to test how the compounds of the present technology behave alone (i.e., without other commercial SPF actives) in cosmetic formula, or in combination with other commercially available SPF actives.

TABLE 5
Formulation A comprising compound of formula 38

| # | | Quantité | Quantité |
|---|---|---|---|
| ingrédient | Nom commercial | (%) | (kg) |
| 1 | Eau | 63.70% | 0.637 |
| 2 | EDTA BD:Disodium EDTA | 0.10% | 0.001 |
| 3 | Glycerin USP | 2.50% | 0.025 |
| 4 | Octocrylene | 3.00% | 0.030 |
| 5 | Finsolv TPP | 4.00% | 0.040 |
| 6 | Avobenzone USP NeoHeliopan AV | 3.00% | 0.030 |
| 7 | Homosalate Neo Heliopan HMS | 8.00% | 0.080 |
| 8 | Jojoba Oil | 2.00% | 0.020 |
| 9 | ARLACEL 165 FP-PA-(RB) | 2.50% | 0.025 |
| 10 | Eumulgin SG | 1.00% | 0.010 |
| 11 | Phenoxyethanol | 0.20% | 0.002 |
| 12 | Spectrastat | 1.00% | 0.010 |
| 13 | Simulgel NS | 3.00% | 0.030 |
| 14 | Compound of formula 38 | 1.00% | 0.010 |
| 15 | Finsolv TPP | 4.00% | 0.040 |
| 16 | Tween 20 | 1.00% | 0.010 |
| | Total= | 100.00% | 1.00 |

TABLE 6
Formulation B comprising compound of formula 38 + Alcohol

| # | | Quantité | Quantité |
|---|---|---|---|
| ingrédient | Nom commercial | (%) | (kg) |
| 1 | Eau | 63.70% | 0.637 |
| 2 | EDTA BD:Disodium EDTA | 0.10% | 0.001 |
| 3 | Glycerin USP | 2.50% | 0.025 |
| 4 | Octocrylene | 3.00% | 0.030 |
| 5 | Finsolv TPP | 4.00% | 0.040 |
| 6 | Avobenzone USP NeoHeliopan AV | 3.00% | 0.030 |
| 7 | Homosalate Neo Heliopan HMS | 8.00% | 0.080 |
| 8 | Jojoba Oil | 2.00% | 0.020 |
| 9 | ARLACEL 165 FP-PA-(RB) | 2.50% | 0.025 |
| 10 | Eumulgin SG | 1.00% | 0.010 |
| 11 | Phenoxyethanol | 0.20% | 0.002 |
| 12 | Spectrastat | 1.00% | 0.010 |
| 13 | Simulgel NS | 3.00% | 0.030 |
| 14 | Compound of formula 38 | 1.00% | 0.010 |
| 15 | Alcool Éthylique | 2.00% | 0.020 |
| 16 | Eau | 3.00% | 0.030 |
| | Total= | 100.00% | 1.00 |

TABLE 7
Formulation C comprising compound of formula 37

| # | | Quantité | Quantité |
|---|---|---|---|
| ingrédient | Nom commercial | (%) | (kg) |
| 1 | Eau | 63.70% | 0.637 |
| 2 | EDTA BD:Disodium EDTA | 0.10% | 0.001 |
| 3 | Glycerin USP | 2.50% | 0.025 |
| 4 | Octocrylene | 3.00% | 0.030 |
| 5 | Finsolv TPP | 4.00% | 0.040 |
| 6 | Avobenzone USP NeoHeliopan AV | 3.00% | 0.030 |
| 7 | Homosalate Neo Heliopan HMS | 8.00% | 0.080 |
| 8 | Jojoba Oil | 2.00% | 0.020 |
| 9 | ARLACEL 165 FP-PA-(RB) | 2.50% | 0.025 |
| 10 | Eumulgin SG | 1.00% | 0.010 |
| 11 | Phenoxyethanol | 0.20% | 0.002 |
| 12 | Spectrastat | 1.00% | 0.010 |
| 13 | Simulgel NS | 3.00% | 0.030 |
| 14 | Compound of formula 37 | 1.00% | 0.010 |
| 15 | Finsolv TPP | 4.00% | 0.040 |
| 16 | Tween 20 | 1.00% | 0.010 |
| | Total= | 100.00% | 1.00 |

TABLE 8
Formulation D comprising compound of formula 37 + Alcohol

| # | | Quantité | Quantité |
|---|---|---|---|
| ingrédient | Nom commercial | (%) | (kg) |
| 1 | Eau | 63.70% | 0.637 |
| 2 | EDTA BD:Disodium EDTA | 0.10% | 0.001 |
| 3 | Glycerin USP | 2.50% | 0.025 |
| 4 | Octocrylene | 3.00% | 0.030 |
| 5 | Finsolv TPP | 4.00% | 0.040 |
| 6 | Avobenzone USP NeoHeliopan AV | 3.00% | 0.030 |
| 7 | Homosalate Neo Heliopan HMS | 8.00% | 0.080 |
| 8 | Jojoba Oil | 2.00% | 0.020 |
| 9 | ARLACEL 165 FP-PA-(RB) | 2.50% | 0.025 |
| 10 | Eumulgin SG | 1.00% | 0.010 |
| 11 | Phenoxyethanol | 0.20% | 0.002 |
| 12 | Spectrastat | 1.00% | 0.010 |
| 13 | Simulgel NS | 3.00% | 0.030 |
| 14 | Compound of formula 37 | 1.00% | 0.010 |
| 15 | Alcool Éthylique | 2.00% | 0.020 |
| 16 | Eau | 3.00% | 0.030 |
| | Total= | 100.00% | 1.00 |

TABLE 9
Formulation E comprising compound of formula 35

| # | | Quantité | Quantité |
|---|---|---|---|
| ingrédient | Nom commercial | (%) | (kg) |
| 1 | Eau | 63.70% | 0.637 |
| 2 | EDTA BD:Disodium EDTA | 0.10% | 0.001 |
| 3 | Glycerin USP | 2.50% | 0.025 |
| 4 | Octocrylene | 3.00% | 0.030 |
| 5 | Finsolv TPP | 4.00% | 0.040 |
| 6 | Avobenzone USP NeoHeliopan AV | 3.00% | 0.030 |
| 7 | Homosalate Neo Heliopan HMS | 8.00% | 0.080 |
| 8 | Jojoba Oil | 2.00% | 0.020 |
| 9 | ARLACEL 165 FP-PA-(RB) | 2.50% | 0.025 |
| 10 | Eumulgin SG | 1.00% | 0.010 |
| 11 | Phenoxyethanol | 0.20% | 0.002 |
| 12 | Spectrastat | 1.00% | 0.010 |
| 13 | Simulgel NS | 3.00% | 0.030 |
| 14 | Compound of formula 35 | 1.00% | 0.010 |
| 15 | Finsolv TPP | 4.00% | 0.040 |
| 16 | Tween 20 | 1.00% | 0.010 |
| | Total= | 100.00% | 1.00 |

TABLE 10
Formulation F comprising compound of formula 35 + Alcohol

| # | | Quantité | Quantité |
|---|---|---|---|
| ingrédient | Nom commercial | (%) | (kg) |
| 1 | Eau | 63.70% | 0.637 |
| 2 | EDTA BD:Disodium EDTA | 0.10% | 0.001 |
| 3 | Glycerin USP | 2.50% | 0.025 |
| 4 | Octocrylene | 3.00% | 0.030 |
| 5 | Finsolv TPP | 4.00% | 0.040 |
| 6 | Avobenzone USP NeoHeliopan AV | 3.00% | 0.030 |
| 7 | Homosalate Neo Heliopan HMS | 8.00% | 0.080 |
| 8 | Jojoba Oil | 2.00% | 0.020 |
| 9 | ARLACEL 165 FP-PA-(RB) | 2.50% | 0.025 |
| 10 | Eumulgin SG | 1.00% | 0.010 |
| 11 | Phenoxyethanol | 0.20% | 0.002 |
| 12 | Spectrastat | 1.00% | 0.010 |
| 13 | Simulgel NS | 3.00% | 0.030 |
| 14 | Compound of formula 35 | 1.00% | 0.010 |
| 15 | Alcool Éthylique | 2.00% | 0.020 |
| 16 | Eau of formula 35 | 3.00% | 0.030 |
| | Total= | 100.00% | 1.00 |

TABLE 11
Formulation G comprising compound of formula 73

| # | | Quantité | Quantité |
|---|---|---|---|
| ingrédient | Nom commercial | (%) | (kg) |
| 1 | Eau | 63.70% | 0.637 |
| 2 | EDTA BD:Disodium EDTA | 0.10% | 0.001 |
| 3 | Glycerin USP | 2.50% | 0.025 |
| 4 | Octocrylene | 3.00% | 0.030 |
| 5 | Finsolv TPP | 4.00% | 0.040 |
| 6 | Avobenzone USP NeoHeliopan AV | 3.00% | 0.030 |
| 7 | Homosalate Neo Heliopan HMS | 8.00% | 0.080 |
| 8 | Jojoba Oil | 2.00% | 0.020 |
| 9 | ARLACEL 165 FP-PA-(RB) | 2.50% | 0.025 |
| 10 | Eumulgin SG | 1.00% | 0.010 |
| 11 | Phenoxyethanol | 0.20% | 0.002 |
| 12 | Spectrastat | 1.00% | 0.010 |
| 13 | Simulgel NS | 3.00% | 0.030 |
| 14 | Compound of formula 73 | 1.00% | 0.010 |
| 15 | Finsolv TPP | 4.00% | 0.040 |
| 16 | Tween 20 | 1.00% | 0.010 |
| | Total= | 100.00% | 1.00 |

TABLE 12
Formulation H comprising compound of formula 73 + Alcohol

| | | Quantité | Quantité |
|---|---|---|---|
| # ingrédient | Nom commercial | (%) | (kg) |
| 1 | Eau | 63.70% | 0.637 |
| 2 | EDTA BD: | 0.10% | 0.001 |
| | Disodium EDTA | | |
| 3 | Glycerin USP | 2.50% | 0.025 |
| 4 | Octocrylene | 3.00% | 0.030 |
| 5 | Finsolv TPP | 4.00% | 0.040 |
| 6 | Avobenzone USP | 3.00% | 0.030 |
| | NeoHeliopan AV | | |
| 7 | Homosalate Neo | 8.00% | 0.080 |
| | Heliopan HMS | | |
| 8 | Jojoba Oil | 2.00% | 0.020 |
| 9 | ARLACEL 165 FP-PA-(RB) | 2.50% | 0.025 |
| 10 | Eumulgin SG | 1.00% | 0.010 |
| 11 | Phenoxyethanol | 0.20% | 0.002 |
| 12 | Spectrastat | 1.00% | 0.010 |
| 13 | Simulgel NS | 3.00% | 0.030 |
| 14 | Compound of formula 73 | 1.00% | 0.010 |
| 15 | Alcool Éthylique | 2.00% | 0.020 |
| 16 | Eau | 3.00% | 0.030 |
| | Total = | 100.00% | 1.00 |

TABLE 13
Formulation I: Basic Neutral Cream comprising
a commercially available SPF active

| # ingrédient | Nom commercial | Quantité (%) | Quantité (kg) |
|---|---|---|---|
| 1 | UV-CUT ZNO 68 CG | 36.00% | 0.360 |
| 2 | Emulium Illustro | 3.50% | 0.035 |
| 3 | Innollient LO | 7.00% | 0.070 |
| 4 | Kester K 60 P | 1.50% | 0.015 |
| 5 | Dub 810 C | 5.50% | 0.055 |
| 6 | Eau | 45.00% | 0.450 |
| 7 | Tristat Eco | 1.50% | 0.015 |
| | Total = | 100.00% | 1.00 |

TABLE 14
Formulation J: Formulation I +
compound of formula 42 in acid form

| | | Quantité | Quantité |
|---|---|---|---|
| # ingrédient | Nom commercial | (%) | (kg) |
| 1 | UV-CUT ZNO 68 CG | 36.00% | 0.360 |
| 2 | Emulium Illustro | 3.50% | 0.035 |
| 3 | Innollient LO | 3.00% | 0.030 |
| 4 | Kester K 60 P | 1.50% | 0.015 |
| 5 | Dub 810 C | 5.50% | 0.055 |
| 6 | Eau | 45.00% | 0.450 |
| 7 | Tristat Eco | 1.50% | 0.015 |
| 8 | Compound of formula | 1.00% | 0.010 |
| | 42 in acid form | | |
| 9 | Innollient LO | 3.00% | 0.030 |
| | Total = | 100.00% | 1.00 |

TABLE 15
Formulation K: Formulation I + compound of formula 46

| | | Quantité | Quantité |
|---|---|---|---|
| # ingrédient | Nom commercial | (%) | (kg) |
| 1 | UV-CUT ZNO 68 CG | 36.00% | 0.360 |
| 2 | Emulium Illustro | 3.50% | 0.035 |
| 3 | Innollient LO | 3.00% | 0.030 |
| 4 | Kester K 60 P | 1.50% | 0.015 |
| 5 | Dub 810 C | 5.50% | 0.055 |
| 6 | Eau | 45.00% | 0.450 |
| 7 | Tristat Eco | 1.50% | 0.015 |
| 8 | Compound of formula 46 | 1.00% | 0.010 |
| 9 | Innollient LO | 3.00% | 0.030 |
| | Total = | 100.00% | 1.00 |

### Example 60: Assessment of UV Absorption by the Protecting Compounds

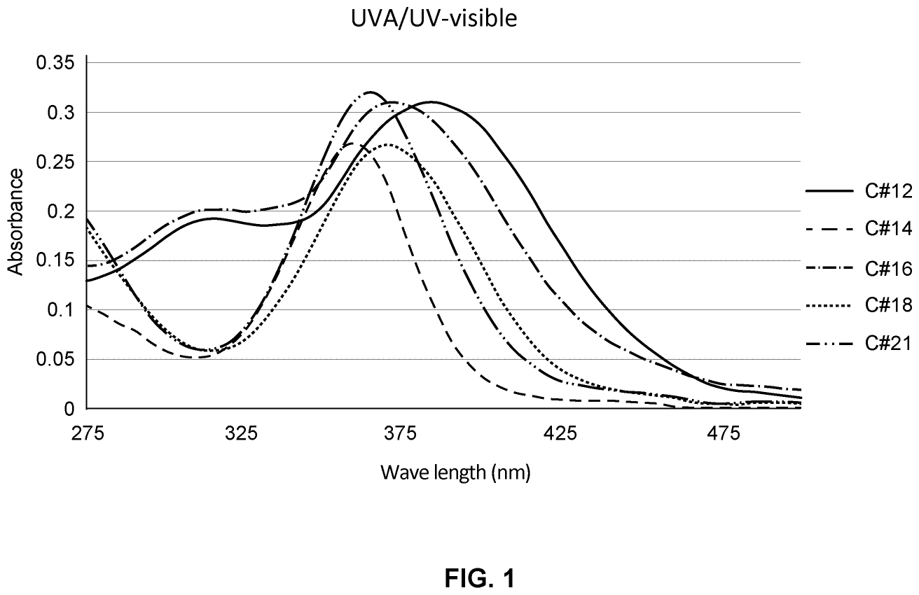

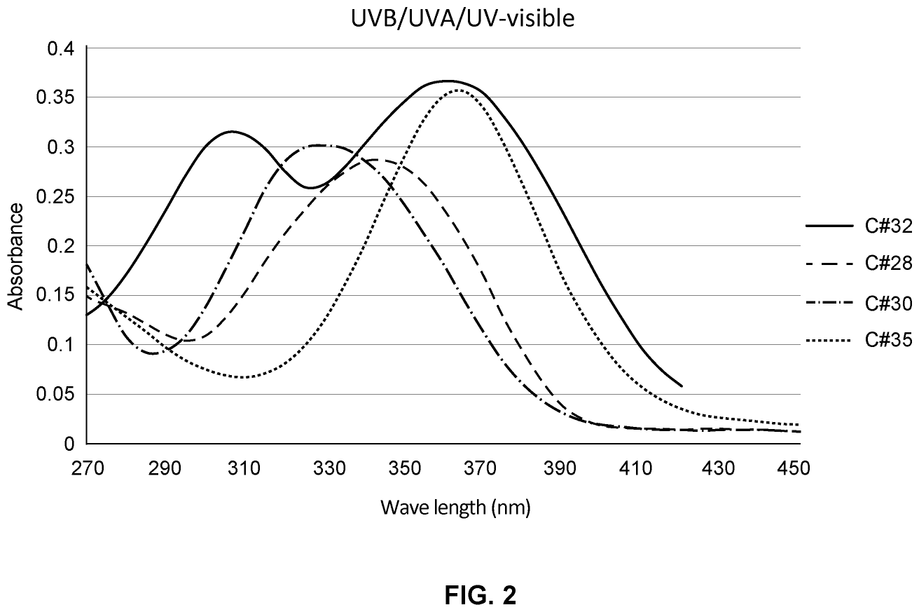

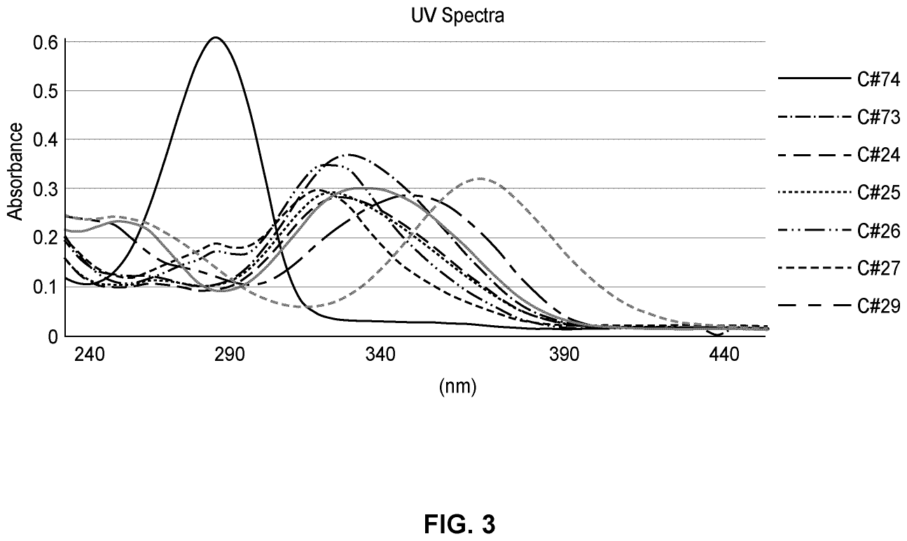

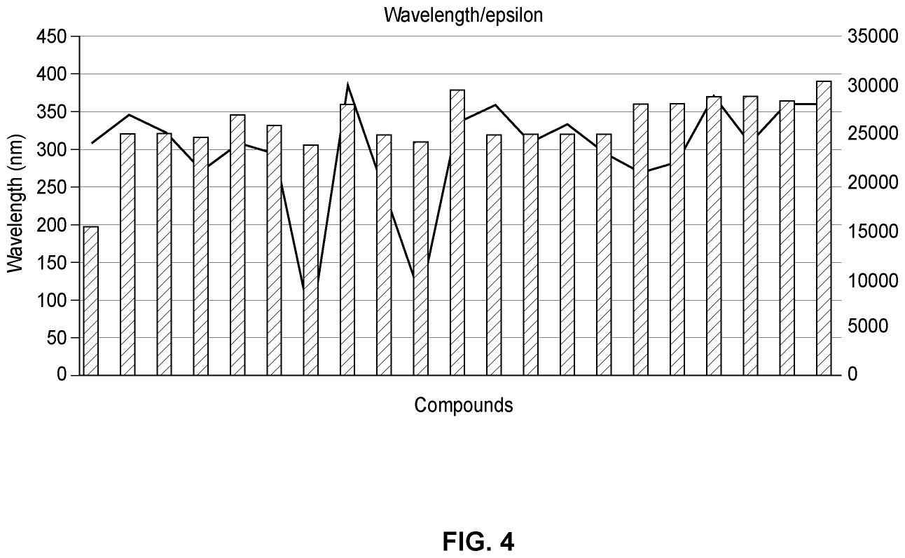

Solutions comprising compounds according to certain embodiments of the present technology were prepared in Ethanol at 5 μg/mL. A sample of each was then analysed on a UV spectrophotometer—Thermofisher evolution type. The data was processed in excel file while spectra were generated. The data generated is presented in FIGS. 1-4.

### Example 61: Assessment of Photostability of Compounds

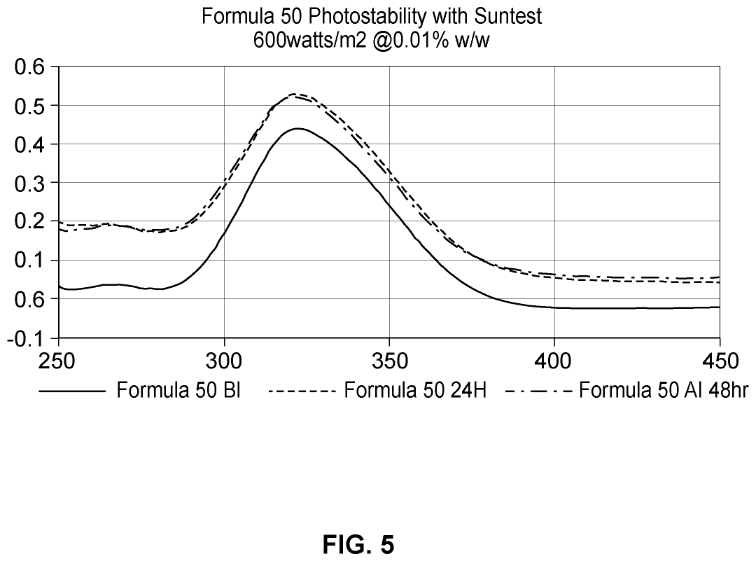

Photostability of a compound of the present technology was assessed by dissolving the compound in Ethanol at 0.01% w/w and exposing the solution to UV radiation using the Suntest equipment at 600 watts/m2. The sample was then taken out and analyzed with a UV-Spectrophotometer after 24 hours and 48 hours. Spectra were overlapped to determine if absorption was lost overtime. The results are shown in FIG. 5.

### Example 62: Assessment of the Thermal Stability of Compounds

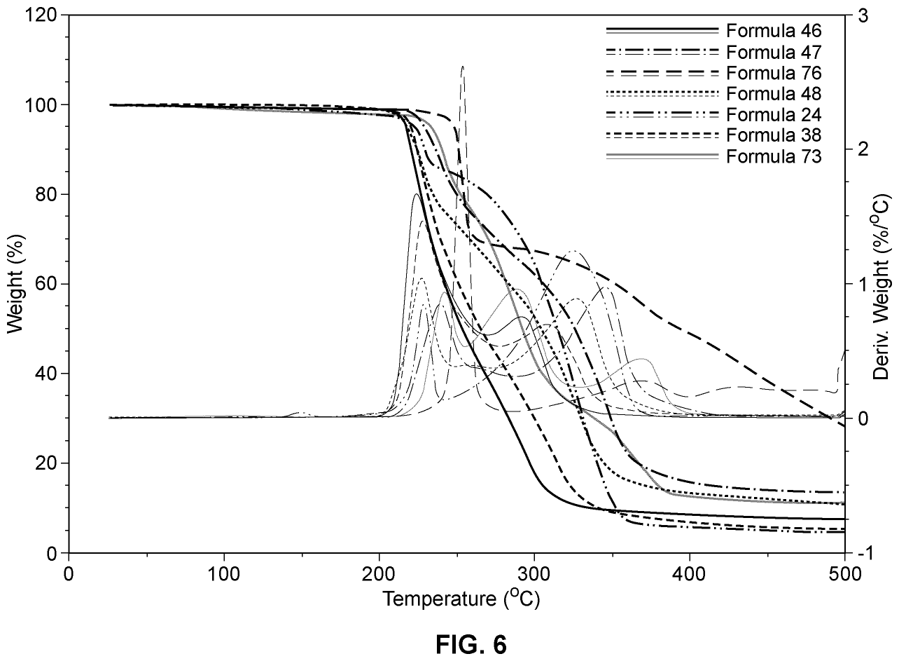

The determine the suitability of the compounds of the present technology for use in different applications, the thermal stability of the compounds, as seen in Table 16 below, were assessed by Thermogravimetric Analysis (TGA). Briefly, the residual weight (%) of the compounds was measured at different temperatures, including 100° C. for water boiling, and other temperatures corresponding to extrusion processes, and the T onset (° C.) by intersection points of tangents was checked. The results are represented in Table 16 below, and in FIG. 6.

TABLE 16
Summary of TGA

| | Initial | Residual weight % at different | | | | T | Td |
|---|---|---|---|---|---|---|---|
| | weight | temperatures (w %) | | | | onset | max |
| Samples | (mg) | 100 | 190 | 210 | 250 | (C) * | (° C.) |
| Formula 77 | 7.0510 | 99.2 | 91.2 | 83.8 | 66.4 | 187.4 | |
| Formula 67 | 7.7850 | 99.4 | 92.9 | 82.0 | 68.1 | 190.5 | |
| Formula 68 | 5.7680 | 97.9 | 93.6 | 87.3 | 75.0 | 197.2 | |
| Formula 69 | 6.0200 | 99.3 | 95.2 | 88.0 | 82.5 | 193.6 | |
| Formula 46 | 5.1400 | 99.4 | 99.1 | 98.4 | 52.4 | 215.2 | |
| Formula 47 | 5.4430 | 99.5 | 99.2 | 99.0 | 79.5 | 226.0 | |
| Formula 76 | 6.4740 | 99.6 | 99.4 | 99.3 | 97.7 | 256.0 | 260.9 |
| Formula 76 | 6.5880 | 99.5 | 99.2 | 99.0 | 91.7 | 248.2 | 253.3 |
| Formula 48 | 5.7840 | 99.2 | 98.8 | 97.8 | 72.9 | 215.7 | |
| Formula 49 | 6.7740 | 99.3 | 97.8 | 97.2 | 70.2 | 215.3 | |
| Formula 60 | 7.1590 | 97.8 | 85.6 | 77.0 | 58.2 | 168.9 | |
| Formula 50 | 6.4280 | 99.2 | 98.1 | 97.9 | 69.3 | 229.5 | |
| Formula 51 | 7.8950 | 99.5 | 96.1 | 85.8 | 65.1 | 188.0 | |
| Formula 52 | 5.1810 | 95.9 | 92.4 | 86.0 | 63.2 | 199.1 | |
| Formula 61 | 5.1560 | 99.0 | 98.2 | 92.6 | 62.1 | 205.0 | |
| Formula 62 | 5.0900 | 94.5 | 85.0 | 75.8 | 54.0 | 186.1 | |
| Formula 63 | 5.6400 | 98.7 | 97.3 | 88.4 | 68.0 | 197.4 | |
| Formula 64 | 11.1900 | 97.2 | 87.7 | 80.3 | 60.9 | 187.0 | |
| Formula 53 | 6.2600 | 99.5 | 98.6 | 86.3 | 40.5 | 199.3 | |
| Formula 54 | 5.7020 | 98.0 | 84.0 | 72.7 | 60.1 | 176.7 | |
| Formula 55 | 6.7390 | 97.8 | 96.4 | 92.2 | 71.2 | 205.0 | |
| Formula 65 | 6.2590 | 95.3 | 91.7 | 89.3 | 80.3 | 200.2 | |
| Formula 56 | 8.2240 | 99.4 | 98.9 | 94.3 | 68.0 | 207.6 | |

### Example 63: Coating Compositions

Coating compositions according to certain embodiments of the present technology, for application on textiles, were prepared as exemplified in Tables 17 and 18. Table 17 exemplifies a composition formulated for application by padding. Briefly, the powdered compounds were dispersed using a mechanical stirrer equipped with a Cowles blade in a solvent for 30 minutes. Once the powder was fully dissolved, the binder and crosslinker were added and mixed.

TABLE 17
Coating composition for padding application

| | Weight concentration |
|---|---|
| Product | (% w/w) |
| Compound of formula 72 | 5.0 |
| Compound of formula 42 | 5.0 |
| Ethanol, Technical grade | 73.0 |
| Water, Deionised | 0 |
| Desmodur 2802, Covestro | 2.0 |
| BYK-190, Atlana | 0 |
| Permax 232, Lubrizol | 15.0 |
| Total | 100 |

TABLE 28
Coating composition for spray application

| | Weight concentration |
|---|---|
| Product | (% w/w) |
| Ethanol, technical grade | 15.0 |
| Compound of formula 1 | 5.0 |
| Compound of formula 72 | 5.0 |
| Water, deionised | 65.0 |
| Citric acid, reagent grade | pH4 |
| DUR-O-SET Elite 22 LV, Celanese | 10.0 |
| Total | 100 |

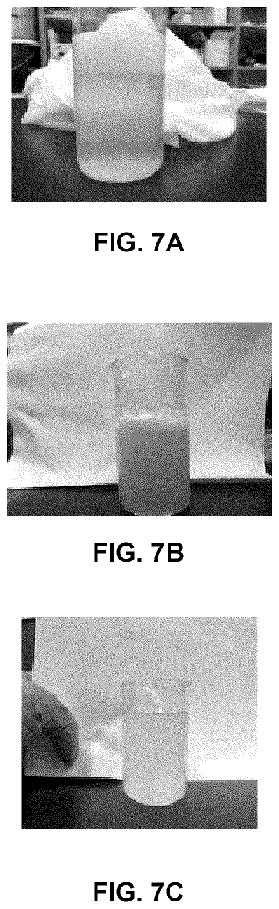

The Coating composition of Table 28 was prepared by mixing the compounds of the present technology in ethanol to form a paste using a magnetic stirrer or a mechanical stirrer equipped with a Cowles Blade. Water was then added to dissolve the paste, followed by the resin and crosslinker. Alternatively, a water-based formulation was prepared by using a high shear mixer (rotor-strator, IKA Turax T-25). First, a polymeric dispersant (Disperbyk-190) was mixed in water. Once dispersed, the powdered compound of the present technology was added to the solution. The powder was dispersed for at least 3 minutes, but no more than 5 min. at 10 000 rpm (FIG. 7A). It is envisioned that a mechanical stirrer with a Cowles blade can achieve similar result when mixing time is over 1 hour. Defoamer BYK-024 and BYK-022 were also added to the formulation at 0.8% to reduce foaming during the mixing operation (FIG. 7B). The formulation was found to be stable for a period of at least two weeks (FIG. 7C).

### Example 64: Application of Coating Composition by Padding

A laminated aramid fabric was immersed in a liquor bath comprising a coating composition as detailed in Table 29 below.

TABLE 29
Coating composition for padding

| | Weight | |
|---|---|---|
| Product | (% m/m) | Function |
| Compound of formula 72 | 5.0 | UV protector |
| Compound of formula 42 | 5.0 | UV protector |
| Ethanol, technical grade | 84.3 | Solvent |
| Water, deionised | 0 | Solvent |
| Desmodur 2802, Covestro | 0.7 | Carbodiimide Crosslinker |
| BYK-190, Atlana | 0 | Dispersant |
| Permax 232, Lubrizol | 5.0 | Polyurethane binder, soft-touch |
| Total | 100 | |

The fabric was then passed through a two-roll padder in front of a pin-tenter frame. The immersion time and the pressure at the rolls is adjusted to obtain a wet pick-up of 100%. After passing through the pad trough, the fabric was squeezed by the pad rollers to remove the excess composition and the fabric was guided onto pin clips on the tenter frame and fixed thereto to prevent shrinking. The tenter frame oven both dried and cured the coating composition onto the fabric. The drying/curing temperature and time were fixed according to each composition and the fabric. After exiting the oven, the fabric was batched on a roll-up device. The parameters for the padding application are summarized in Table 30 below.

TABLE 30
Padding parameters
Padding parameter
Padding

| Pressure | 5 to 10 | kPa |
|---|---|---|
| Wet pick-up (%) | 100 | |
| Dry pick-up (%) | 16 | |
| Line speed | 1 | m/min. |

Oven 1

| | | |
|---|---|---|
| Temperature | 70° | C. |

Oven 2

| | | |
|---|---|---|
| Temperature | 70° | C. |

The fabric was then aged by UV according to AATCC 169-Note 1 Option 4 as presented in Table 31, for about 150 hours.

TABLE 31
UV aging parameters

| | Option 1 | Option 2 | Option 3 | Option 4 |
|---|---|---|---|---|
| Black panel | 77 ± 3° C. | | | 63 ± 3° C. |
| temperature | (170 ± 5° F.) | | | (145 ± 5° F.) |
| Relative | 70 ± 5% | | 27 ± 3% | 50 ± 5% |
| humidity | | | | |
| Light only | 90 mins | 60 mins | 120 mins | 102 mins |
| exposure | | | | |
| cycle part | | | | |
| Wetting cycle | 30 mins of | 60 mins of | No water | 18 mins of |
| part | light and | dark period | spray | light and |
| | water spray | No water | Continuous | water spray |
| | | spray | light | |

| | |
|---|---|
| Level of | 0.35 ± 0.01 |
| irradiance | W/m²nm |
| at 340 nm | |
| Level of | 40 ± 0.01 |
| irradiance | W/m² |
| at 300-400 nm | |
| pH of the water | 7 ± 1 |
| Temperature of | 16 ± 5° C. |
| the water | (61 ± 9° F.) |

The breaking strength of the fabrics in the weft direction was then measured when the film was broken. The final load value (N) measured by a cell is then divided by the section (b1×h) to give the breaking strength labeled in MPa. The summary of the results is shown in Table 32 below. Treatment with the compounds of the present technology was shown to improve the breaking strength of the laminated aramid by 22%. The resistance was preserved even after 150 hours of UV aging in the chain direction.

TABLE 32
Comparison of breaking strengths of aramid laminates coated with
the compounds of the present technology, before and after aging.

| Aging | Specification | Aramide | Aramide +C |
|---|---|---|---|
| Before | Breaking Strength | 181.5 ± 13.7 | 233.4 ± 10.8 |
| After | | 151.6 ± 7.3 | 228.2 ± 10.9 |
| | Variation % | 16% | 2% |

### Example 65: Application of Coating Composition by Spraying

A nylon carpet was sprayed with the coating composition of Table 28 using a commercial sprayer to grain 50% of weight on the textile. The fabric was hung to dry overnight. The formulation was applied to provide temporary UV protection, and more specifically against UVC, as a way of decontaminating viruses and providing for resistance against abrasion. The nylon carpets were aged by UVC. Irradiation was performed for 7 days, with a total estimated dose of 90,720,000 μJ/cm². The results demonstrate that treatment with the coating composition of the present technology increases abrasion resistance by 18% after UVC aging compared to a loss of 13% for the carpet without treatment (Table 33).

TABLE 33
Results of abrasion resistance test

| | Abrasion resistance of textile |
|---|---|
| Sample | fabrics (rotary platform) |
| After UVC aging with treatment | 5063 ± 921 |
| After UVC aging without treatment | 3688 ± 375 |
| Reference | 4175 ± 538 |

The composition was further spayed on a wool/polyester blue fabric, which is generally used for airplane seats. The performance of the fabric was assessed before and after UVC aging, with or without protection with the compositions of the present technology. The results show that without protection the wool/polyester fabric loses 11% of its mechanical performance after a year of decontaminations with 1 decontamination cycle per day by a robot emitting a UVC irradiance of 480 μW/cm² for 10 minutes/cycle. A summary of the results are presented in Table 34 below.

TABLE 34
Results of performance of wool/polyester
fabric with or without protection

| | | Warp direction | |
|---|---|---|---|
| UVC Conditions | Samples | B. Force (N) | E. at break (%) |
| Before UVC aging | Reference | 449.7 ± 6.9 | 51.3 ± 1.8 |
| After UVC aging | Reference | 400.7 ± 9.4 | 42.6 ± 1.0 |
| | C#1 (5%) + | 443.9 ± 12.1 | 53.3 ± 3.3 |
| | C#17(5%) | | |

### Example 66: Application of Coating Composition with Dye Bath

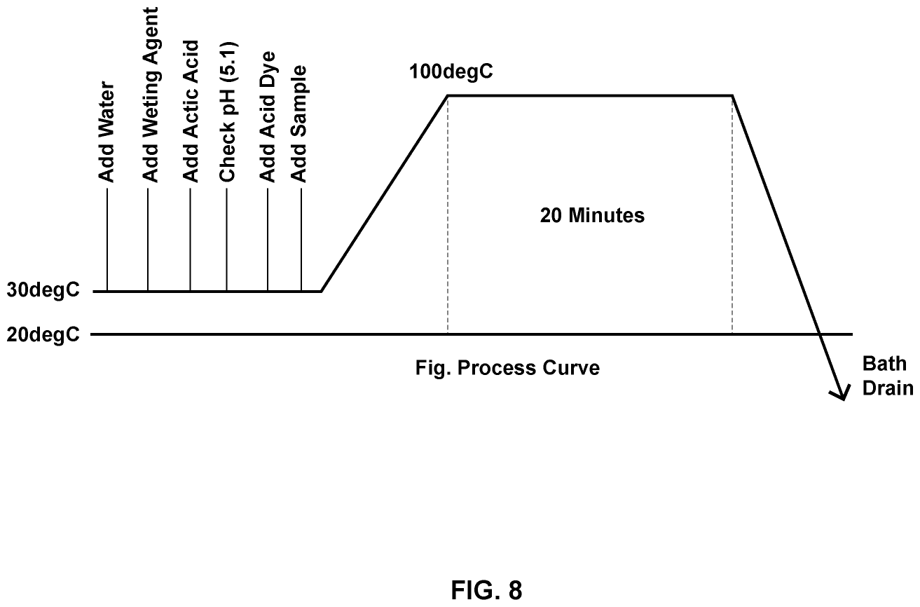

Compounds of the present technology were added to a dye bath (or vat) along with water, and a 1% sodium dodecyl sulfate solution, and mixed with high shear Turax for 30 sec at 10 000 rpm. Acetic acid at a concentration of 1% was then added and stirred. The pH was measured to be between 4.5 to 5.5. Additional dyes were added and the temperature of the mixture was measured. The fabric was then added to the vat and treated per the protocol shown in FIG. 8. The fabric was then removed from the cooled vat, rinsed with cold and hot water and spun to remove excess water. Finally, the fabric was dried in an air oven at 50 degrees Celsius.

### Example 67: Integration of Coating Composition by Extrusion

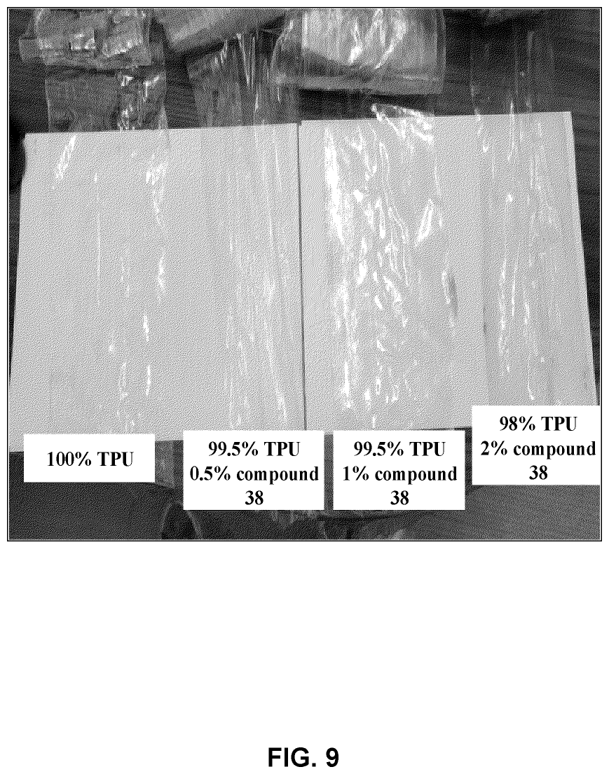

The compounds of the present technology were integrated into textiles and fabrics to limit the aging of the polymer materials following UV exposure. The composition used for extrusion comprised between about 0.5 w % to about 2 w % of the compounds of the present technology. The extrusion process was carried out using a Thermofisher 11 parallel twin-screw extruder configured with the following parameters: mono-vis feeder—process 11-melt pump—flat die 150 mm. An aromatic thermoplastic polyurethane (TPU) elastomer was used as a model, as the presence of the aromatic functions implies discoloration. The TPUs used were TPU from Irogran and Luvosint X92A-1 which is an ester based thermoplastic polyurethane. The experiments were conducted with both pellets or powdered TPU. The compounds of the present technology were integrated by a continuous process either through another feeder or by mixing with masterbatch pellets comprising about 10 w % of the compounds of the present technology. The resulting fabrics obtained comprising varying amounts of TPU and compounds of the present technology are presented in FIG. 9. The thickness of the fabrics obtained by the extrusion method presented herein is provided is Table 35 below.

TABLE 35
Thickness of fabrics obtained by extrusion

| Compound 38 | | | | |
|---|---|---|---|---|
| (wt %) | 0 | 0.5 | 1.0 | 2.0 |
| Mean (μm) | 150 | 126 | 167 | 164 |
| SD (μm) | 15 | 28 | 10 | 15 |
| CV (%) | 10 | 22 | 6 | 9 |

The fabrics obtained were then aged for 40 h in a Xenon chamber according to AATCC 16.3 “Test Method for Colorfastness to Light: Xenon-Arc” (incorporated herein by referent). The physical properties of the fabrics, and more specifically the tensile strength was then determined according to Standard ISO 527-3 “Plastics-determination of tensile properties” proof type 5 (incorporated herein by reference), using a dynamometer at a constant speed rate of 50 mm/min. Although addition of the compounds of the present technology at about 0.5% to about 2% had no or very low influence on the initial tensile properties of the fabrics obtained, a significant difference in tensile strengths was observed after aging. The results of the tensile strengths after aging are summarized in Table 36 below.

TABLE 36
Variations in tensile strengths following aging
of the fabrics with or without the compound
of formula 38 (C38) of the present technology

| | Stress at Break (MPa) | Elongation at break (%) |
|---|---|---|
| TPU | −20% | −15% |
| 99.5% TPU + | −5% | −10% |
| 0.5% Compound 38 | | |

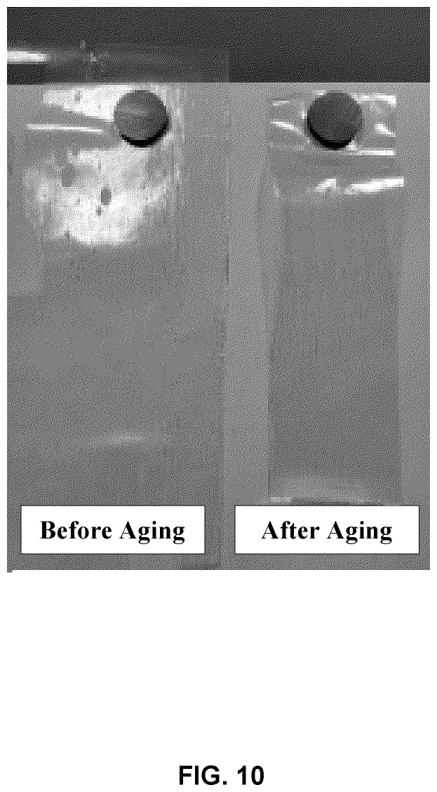

The ability of the fabrics to change color following after aging was further measured using a spectrophotometer Datacolor 850, observation 10° according to ASTM E313: “Standard Practice for Calculating Yellowness and Whiteness Indices from Instrumentally Measured Color Coordinates” (incorporated herein by reference). The results are demonstrated in FIG. 10 and summarized in Table 33 below. As seen in Table 37 below, an increase of the initial yellow index was seen with increasing content of the compounds of the present technology. The yellow index further increased significantly after aging in all samples. The compounds of the present technology did not significantly prevent increases in the yellow index following aging regardless of their content in the fabric.

TABLE 37
Variation of color following aging of
the fabrics of the present technology

| Compound 38 | 0 | 0.56 | 1.0 | 2.0 |
|---|---|---|---|---|
| Yellow index initial | −0.57 | −0.10 | 1.97 | 1.26 |
| Yellow index after | 8.61 | 9.23 | 9.7 | 7.02 |
| aging | | | | |
| ΔE after aging | 5.61 | 5.72 | 5.30 | 3.54 |
| ΔE/initial ref (0) | / | 0.32 | 3.62 | 1.21 |
| ΔE/aged ref (0) | / | 0.42 | 0.80 | 0.91 |

All references cited in this specification, and their references, are incorporated by reference herein in their entirety where appropriate for teachings of additional or alternative details, features, and/or technical background.

While the disclosure has been particularly shown and described with reference to particular embodiments, it will be appreciated that variations of the above-disclosed and other features and functions, or alternatives thereof, may be desirably combined into many other different systems or applications. Also, that various presently unforeseen or unanticipated alternatives, modifications, variations or improvements therein may be subsequently made by those skilled in the art which are also intended to be encompassed by the following claims.

1. A compound having formula I or an acceptable salt thereof: 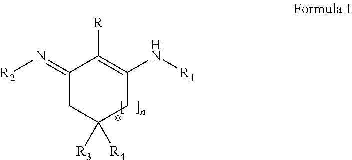
wherein R₁, is an alkyl; an alkene; an alkyne; an aryl; a heterocycle; a cycloalkyl; an alkoxy; an alkanoyl; an arylalkyl; a carboxyl; a heteroaryl; a heteroarylalkyl; a phenyl; a benzyl; a hydroxyl; a carboxylic acid; an ester; a sulfinyl; a sulfhydryl; a sulfide; a sulfonyl; a sulfino; a phosphino; a phosphono; a phosphate; an amine; a halo; a carboxamide, or R₁ forms a heterocycle with R₅;
R₂ is an alkyl; an alkene; an alkyne; an aryl; a heterocycle; a cycloalkyl; an alkoxy; an alkanoyl; a hydroxyl; a halo; a phenyl; a benzyl; a carboxylic acid or an ester group, wherein R₂ comprises at least one nitrogen atom;
R₃ and R₄ are each independently a hydrogen; an alkyl; an alkene; an alkyne; an aryl; a heterocycle; a cycloalkyl; an alkoxy; an alkanoyl; a sulfo group; a hydroxyl group; a phosphono group; an ester group; a carboxylic acid group; or a phenyl group;
R₅ is a hydrogen; an alkyl; an alkene; an alkyne; an aryl; a heterocycle; a cycloalkyl; an alkoxy; an alkanoyl; a alkynyl; a hydroxyl; a sulfo group; a halo group; a phosphono group; an ester group; a carboxylic acid group; a phenyl group; an alkyl fatty acid chain or polyether; and
n is 1,2,3 or 4.

2. The compound of claim 1, wherein the compound has formula:

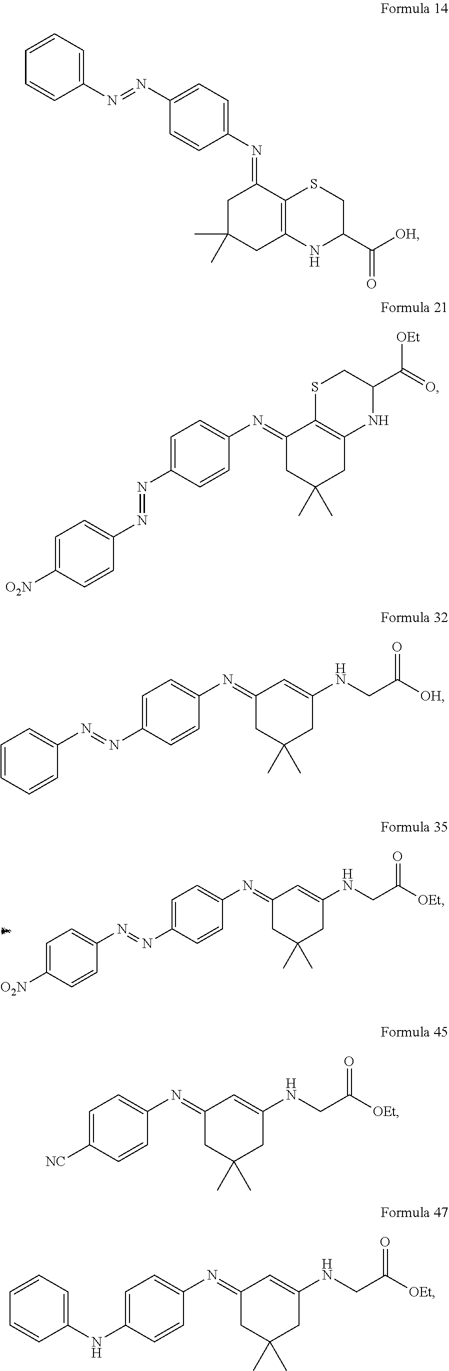 [SMILES: CC1(C)CC2=C(SCC(C(=O)O)N2)/C(=N/c2ccc(/N=N/c3ccccc3)cc2)C1 ; CCOC(=O)C1CSC2=C(CC(C)(C)C/C2=N\c2ccc(/N=N/c3ccc([N+](=O)[O-])cc3)cc2)N1 ; CC1(C)CC(NCC(=O)O)=C/C(=N/c2ccc(/N=N/c3ccccc3)cc2)C1 ; CCOC(=O)CNC1=C/C(=N/c2ccc(/N=N/c3ccc([N+](=O)[O-])cc3)cc2)CC(C)(C)C1 ; CCOC(=O)CNC1=C/C(=N/c2ccc(C#N)cc2)CC(C)(C)C1 ; CCOC(=O)CNC1=C/C(=N/c2ccc(Nc3ccccc3)cc2)CC(C)(C)C1]

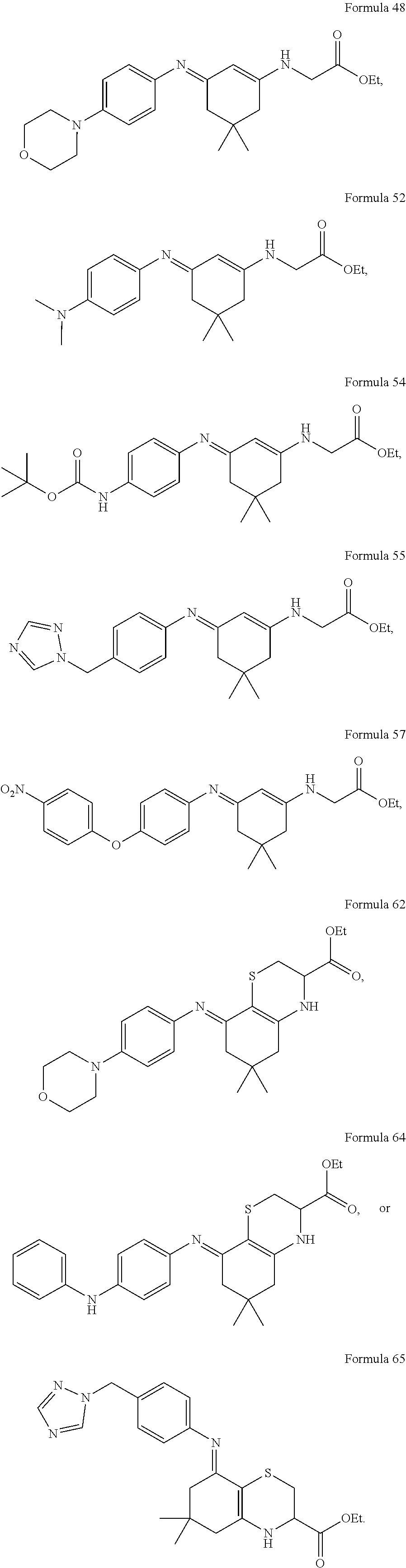

3.-21. (canceled)

22. A method for protecting a surface of a biological or non-biological material against UV radiation and/or visible radiation comprising applying to the surface of said biological or non-biological material a composition comprising the compound of claim 1.

23.-32. (canceled)

33. The method of claim 22, wherein the composition further comprises one or more suitable additives, or one or more UV-blocking agents, or combinations thereof.

34. The method of claim 33, wherein the one or more additives are animal or vegetable fats, waxes, paraffins, starch, tragacanth, cellulose derivatives, polyethylene glycols, silicones, bentonites, silica, talc, zinc oxide, lactose, talc, silica, aluminium hydroxide, calcium silicate, polyamide powder, chlorofluorocarbons, propane/butane, dimethyl ether, water, ethanol, isopropanol, ethyl carbonate, ethyl acetate, benzyl alcohol, benzyl benzoate, propylene glycol, 1,3-butylglycol, oils, cotton seed oil, peanut oil, wheatgerm oil, olive oil, castor oil, sesame oil, glycerol fatty acid ester, polyethylene glycols, fatty acid esters of sorbitan, alkali metal salts of fatty acids, salts of fatty acid mono esters, fatty acid protein hydrolysates, isethionates, lanolin, fatty alcohol, vegetable oils, plant extracts, glycerol, sugars, fatty acid esters, fatty alcohols, silicone oils, paraffin oils, lanolin oils, octyldodecyl oleate, octyldodecyl stearoyl stearate, and polyhydroxystearic acid, octyldodecanol, Emulium Illustro, coco-caprylate/caprate, glyceryl caprylate, glyceryl undecylenate and any combination thereof.

35. The method of claim 22, wherein the biological material is skin

36. The method of claim 22, wherein the personal care product is a sunscreen composition.

37. The method of claim 22, wherein the non-biological composition is a textile or fabric.

38. The method of claim 38, wherein the composition is a coating composition.

39. The method of claim 38, wherein the composition comprises one or more additives selected from an organic solvent, an aqueous solvent, a binder, a surfactant, a wetting agent, a dispersing agent, a cross-linking agent, and a combination thereof.

40. The method of claim 22, wherein the composition comprises the compound of claim 1 or claim 2, or any combination thereof in an amount of between about 0.1% and about 25.0% of the weight of the composition.
